(19) United States
(12) Patent Application Publication
(10) Pub. No.: US 2012/0282331 A1
(43) Pub. Date: Nov. 8, 2012

(54) MAMMALIAN METABOLITES OF STEROIDS

(75) Inventors: Scott C. Chappel, Milton, MA (US); David S. Casebier, Carlisle, MA (US)

(73) Assignee: TOKAI PHARMACEUTICALS, INC., Cambridge, MA (US)

(21) Appl. No.: 13/508,726

(22) PCT Filed: Nov. 9, 2010
(86) PCT No.: PCT/US10/55996

§ 371 Date: Jul. 25, 2012

Related U.S. Application Data
(60) Provisional application No. 61/261,262, filed on Nov. 13, 2009.

(51) Int. Cl. A61K 31/58; A61K 9/48; A61P 35/00; C07J 43/00

(52) U.S. Cl. 424/451; 540/95; 514/176

(57) ABSTRACT

Described herein, in certain embodiments, are steroidal derivatives, methods of making such compounds, pharmaceutical compositions and medicaments comprising such compounds, and methods of using such compounds to treat androgen receptor mediated diseases or conditions.

### FIELD OF THE INVENTION

Compounds, methods of making such compounds, pharmaceutical compositions and medicaments comprising such compounds, and methods of using such compounds to treat androgen receptor mediated diseases or conditions are described.

### BACKGROUND OF THE INVENTION

Prostate cancer is the most common cancer in men and will be responsible for more than 27,360 deaths in 2009 (National Cancer Institute, 2009). The majority of prostate cancer deaths are due to the development of metastatic disease that is unresponsive to conventional androgen deprivation therapy. Androgen deprivation therapy has been the standard of care in patients with prostate cancer since the 1940s. Despite androgen deprivation, most patients ultimately experience disease progression. For many years this later phase of the disease was called “hormone insensitive prostate cancer” or “androgen independent prostate cancer.” It has since become clear that the prostate cancer that emerges after years of androgen deprivation therapy remains dependent upon androgen. The prostate cancer cells that have survived have gained the ability to import low levels of circulating androgens (expressed from adrenal glands), become much more sensitive to these low levels of testosterone, and actually synthesize testosterone within the prostate cancer cell itself. This stage of prostate cancer is now termed “castration resistant prostate cancer” or CRPC.

### SUMMARY OF THE INVENTION

Described herein, in certain embodiments, are compounds, methods of making such compounds, pharmaceutical compositions and medicaments comprising such compounds, and methods of using such compounds to treat androgen receptor mediated diseases.

In some embodiments, the invention provides a compound or a pharmaceutically acceptable salt or N-oxide of a compound having the structure of Formula (1)

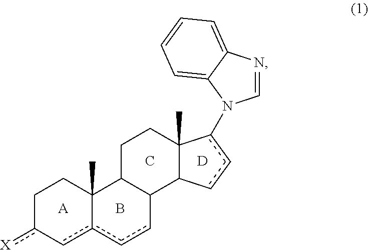

wherein,
(a) the ABCD ring structure and/or one or both methyl groups are independently optionally substituted with one or more substituents selected from C₁-C₆-alkyl, halogenated C₁-C₆-alkyl, C₁-C₆-alkenyl, halogenated C₁-C₆-alkenyl, halogen, amino, aminoalkylene, hydroxyimino, n,n+1-epoxy, carbonyl (oxo), glucuronido, glucuronato, O-linked sulfate, and hydroxy;
(b) X is glucuronido, glucuronato, O-linked sulfate, OH or O; and
(c) dashed lines are taken at each occurrence independently to be double or single bonds, wherein the compound is not:

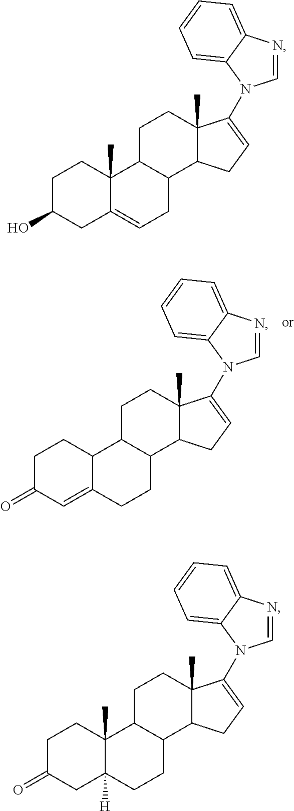

and wherein the compound is formed in vivo after administration of a drug to a subject.

In some embodiments, the invention provides a pharmaceutical composition comprising an effective amount of a compound, wherein after administration of the composition to a subject, the compound produces a metabolite or a pharmaceutically acceptable salt or N-oxide thereof of Formula (1)

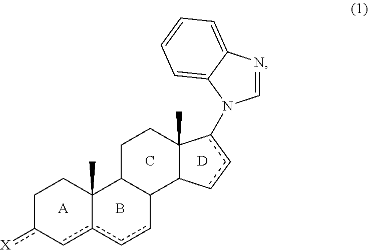

wherein,
(a) the ABCD ring structure and/or one or both methyl groups are independently optionally substituted with one or more substituents selected from C₁-C₆-alkyl, halogenated C₁-C₆-alkyl, C₁-C₆-alkenyl, halogenated C₁-C₆-alkenyl, halogen, amino, aminoalkylene, hydroxyimino, n,n+1-epoxy, carbonyl (oxo), glucuronido, glucuronato, O-linked sulfate, and hydroxy;
(b) X is glucuronido, glucuronato, O-linked sulfate, OH or O; and
(c) dashed lines are taken at each occurrence independently to be double or single bonds, wherein neither the compound nor the metabolite are:

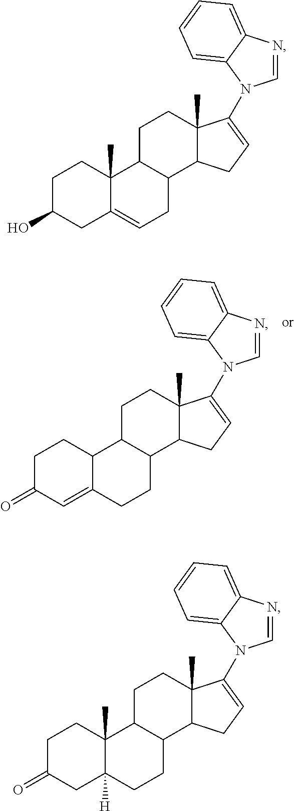

wherein the metabolite is effective for treating an androgen receptor mediated disease or condition.

In some embodiments, the invention provides a method of treating an androgen receptor mediated disease or condition, which method comprises administering to a patient in need thereof a therapeutically effective amount of a compound or a pharmaceutically acceptable salt or N-oxide thereof to inhibit androgen biosynthesis, inhibit androgen receptor signaling and decrease androgen receptor sensitivity, wherein said compound produces a metabolite or a pharmaceutically acceptable salt or N-oxide thereof after administration of the compound to a subject, wherein the metabolite has the structure of Formula (1),

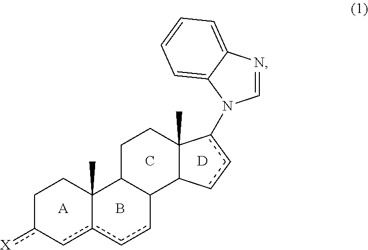

wherein,
(a) the ABCD ring structure and/or one or both methyl groups are independently optionally substituted with one or more substituents selected from C₁-C₆-alkyl, halogenated C₁-C₆-alkyl, C₁-C₆-alkenyl, halogenated C₁-C₆-alkenyl, halogen, amino, aminoalkylene, hydroxyimino, n,n+1-epoxy, carbonyl (oxo), glucuronido, glucuronato, O-linked sulfate, and hydroxy;
(b) X is glucuronido, glucuronato, O-linked sulfate, OH or O; and
(c) dashed lines are taken at each occurrence independently to be double or single bonds, wherein neither the compound nor the metabolite is:

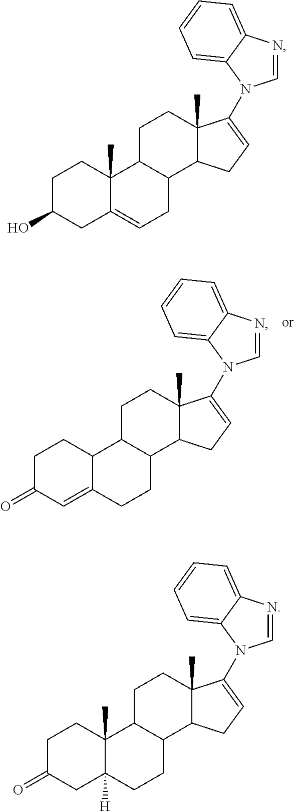

In some embodiments, the invention provides a compound or a pharmaceutically acceptable salt or N-oxide of a compound having the structure of Formula (1)

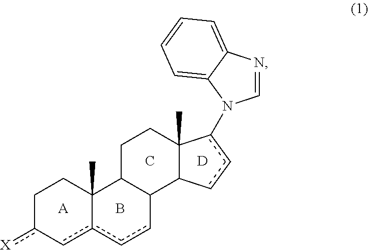

wherein,
(a) the ABCD ring structure and/or one or both methyl groups are independently substituted with two substituents selected from n,n+1 epoxy, oxo, and hydroxy;
(b) X is glucuronido, glucuronato, O-linked sulfate, OH or O; and
(c) dashed lines are taken at each occurrence independently to be double or single bonds, wherein the compound is not:

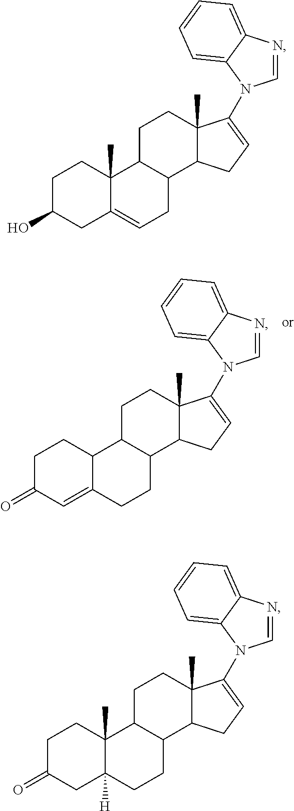

and wherein the compound is formed in vivo after administration of a drug to a subject.

In some embodiments, the invention provides a compound or a pharmaceutically acceptable salt or N-oxide of a compound having the structure of Formula (1)

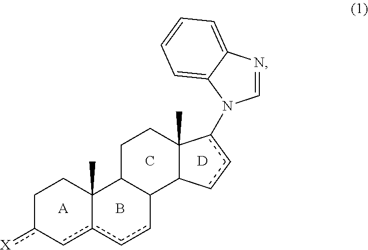

wherein,
(a) the ABCD ring structure and one methyl group is independently substituted with a substituent selected from n, n+1 epoxy, oxo, and hydroxy;
(b) X is glucuronido, glucuronato, O-linked sulfate, OH or O; and
(c) dashed lines are taken at each occurrence independently to be double or single bonds, wherein the compound is not:

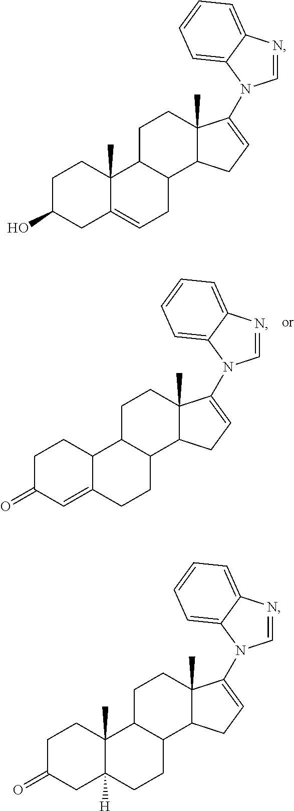

and wherein the compound is formed in vivo after administration of a drug to a subject.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a table of Compound (1) concentration and its potential metabolites following incubations of 10 μm Compound (1) with 0.1M phosphate buffer and pooled rat liver microsomes in the presence and in the absence of NADPH-generating system at various timepoints.

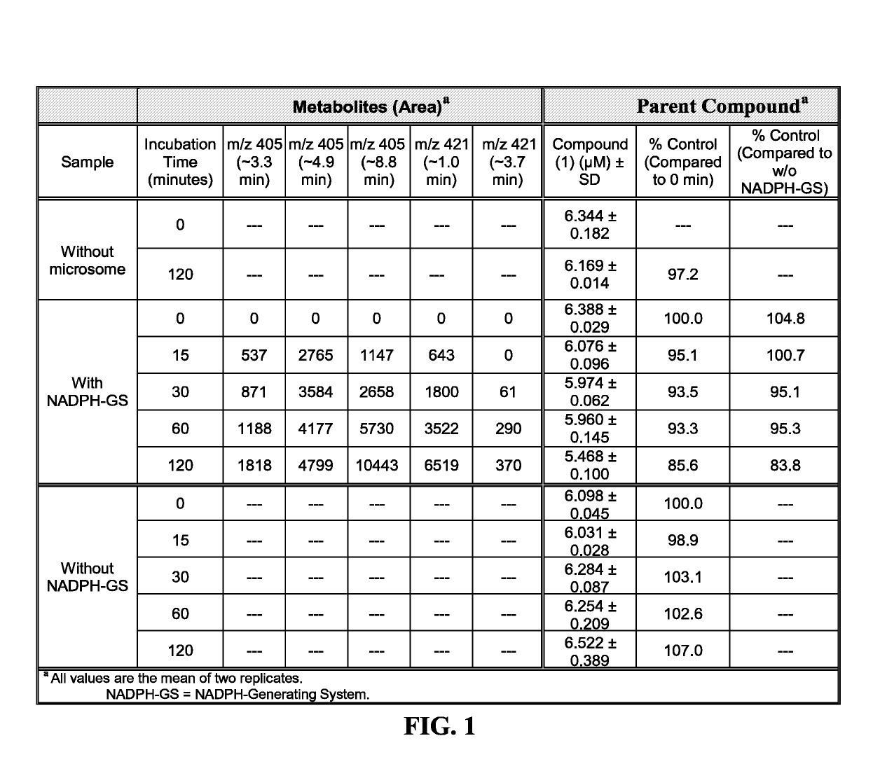

FIG. 2 is a table of Compound (1) concentration and its potential metabolites following incubations of 10 μM Compound (1) with 0.1 M phosphate buffer, 3 mM MgCl₂, 1 mM EDTA and pooled rat liver microsomes in the presence and in the absence of NADPH-generating system at various timepoints.

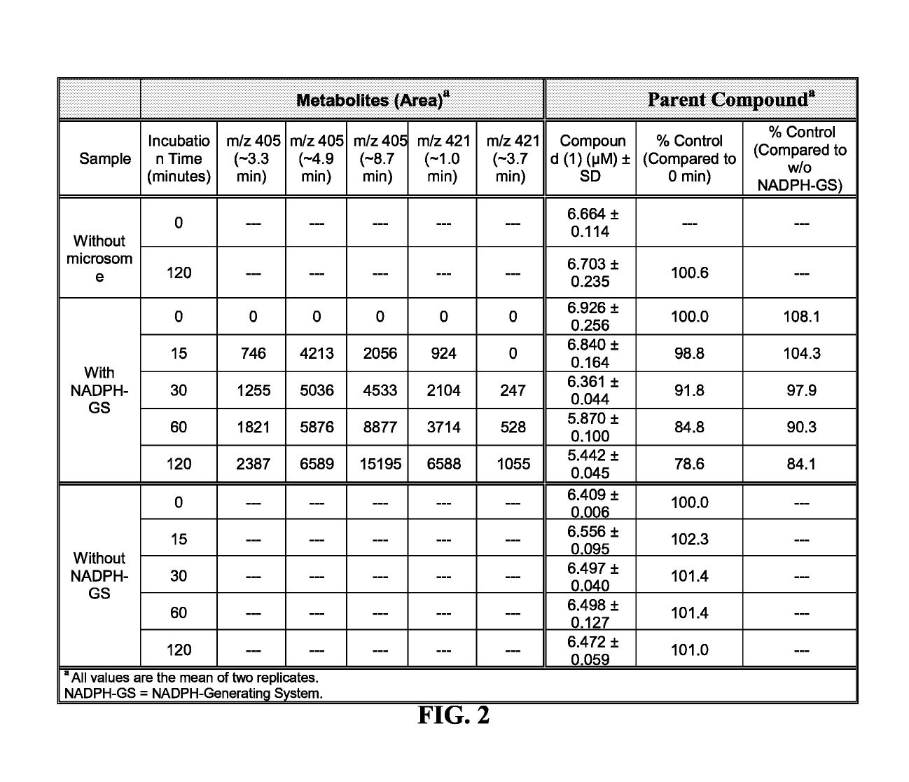

FIG. 3 is a table of Compound (1) concentration and its potential metabolites following incubations of 10 μM Compound (1) with 0.1 M phosphate buffer and pooled dog liver microsomes in the presence and in the absence of NADPH-generating system at various timepoints.

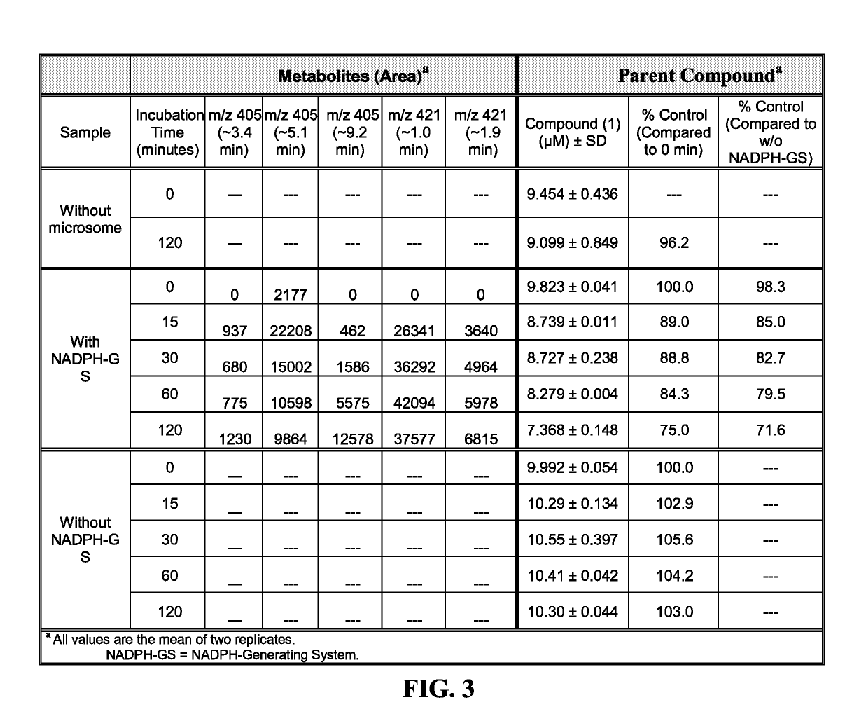

FIG. 4 is a table of Compound (1) concentration and its potential metabolites following incubations of 10 μM Compound (1) with 0.1 M phosphate buffer, 3 mM MgCl₂, 1 mM EDTA and pooled dog liver microsomes in the presence and in the absence of NADPH-generating system at various timepoints.

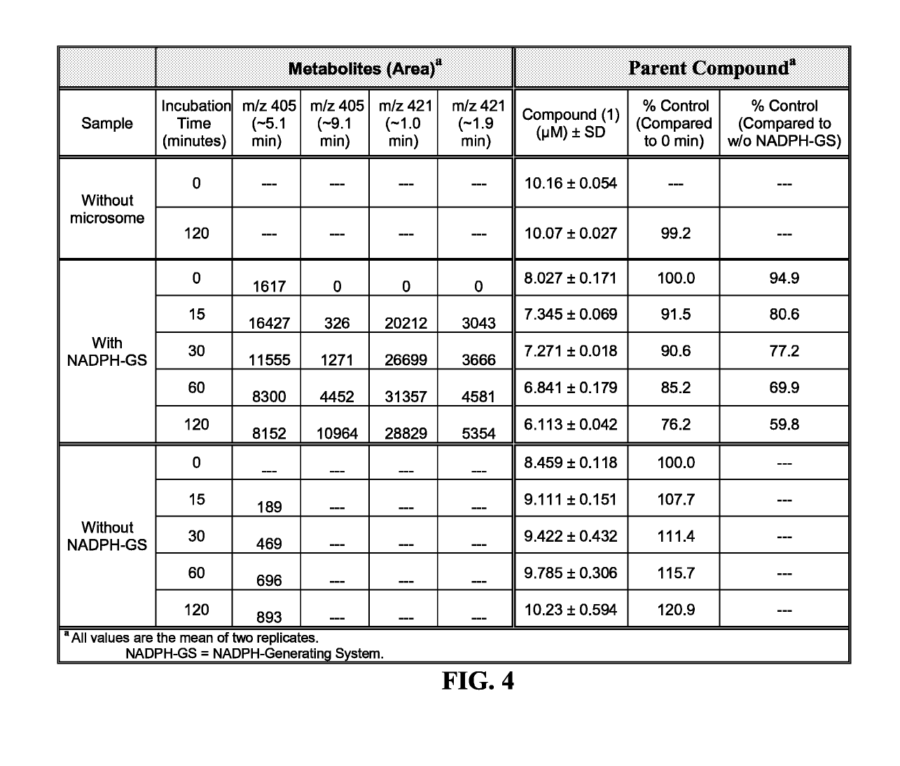

FIG. 5 is a table of Compound (1) concentration and its potential metabolites following incubations of 10 μM Compound (1) with 0.1 M phosphate buffer and pooled monkey liver microsomes in the presence and in the absence of NADPH-generating system at various timepoints.

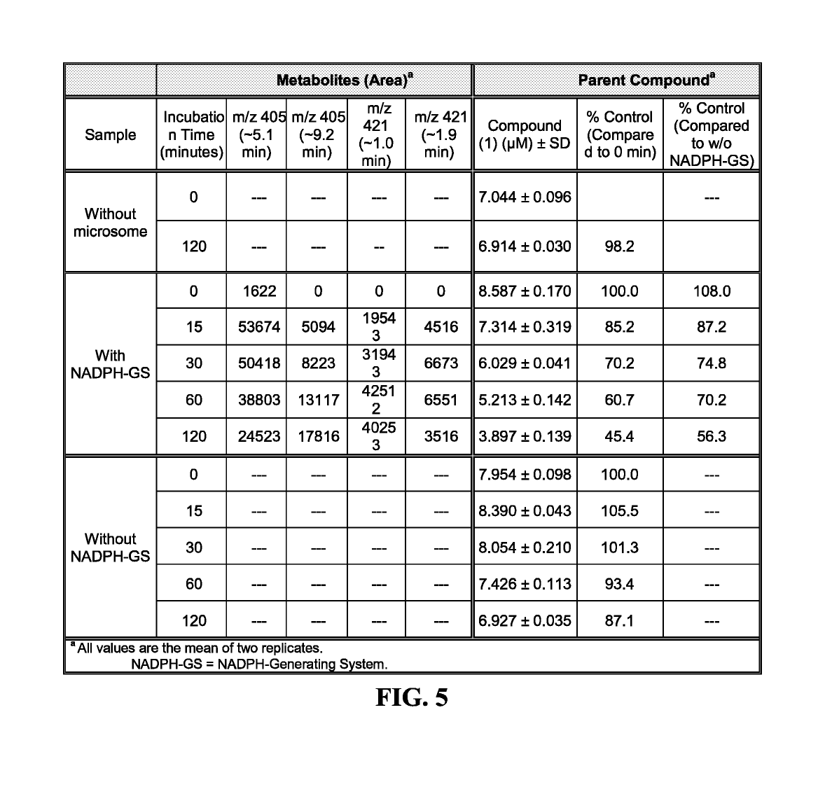

FIG. 6 is a table of Compound (1) concentration and its potential metabolites following incubations of 10 μM Compound (1) with 0.1 M phosphate buffer, 3 mM MgCl₂, 1 mM EDTA and pooled monkey liver microsomes in the presence and in the absence of NADPH-generating system at various timepoints.

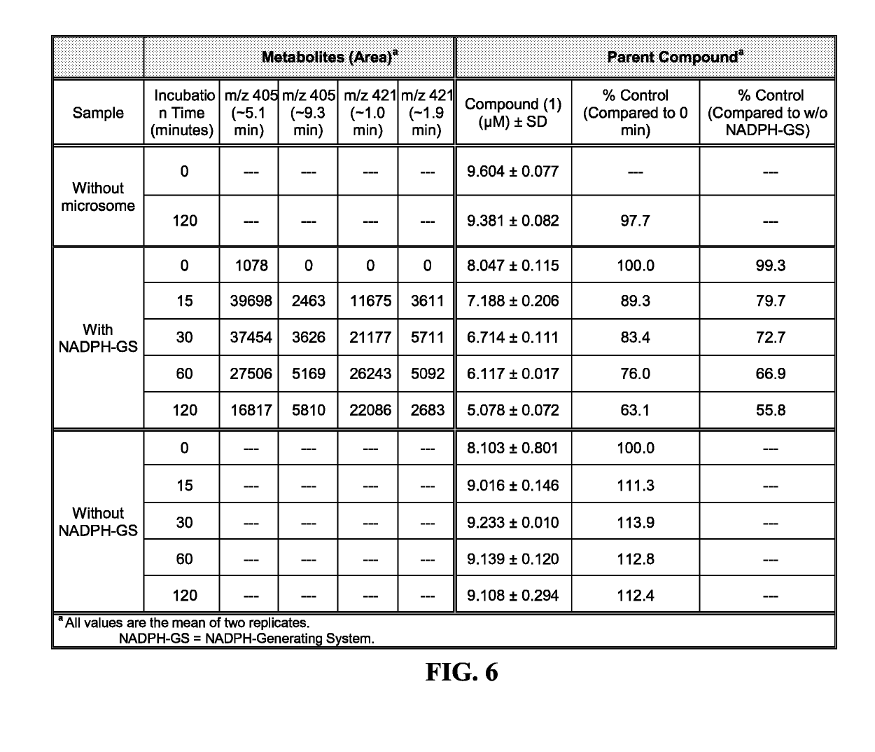

FIG. 7 is a table of Compound (1) concentration and its potential metabolites following incubations of 10 μM Compounds (1) with 0.1 M phosphate buffer and pooled human liver microsomes in the presence and in the absence of NADPH-generating system at various timepoints.

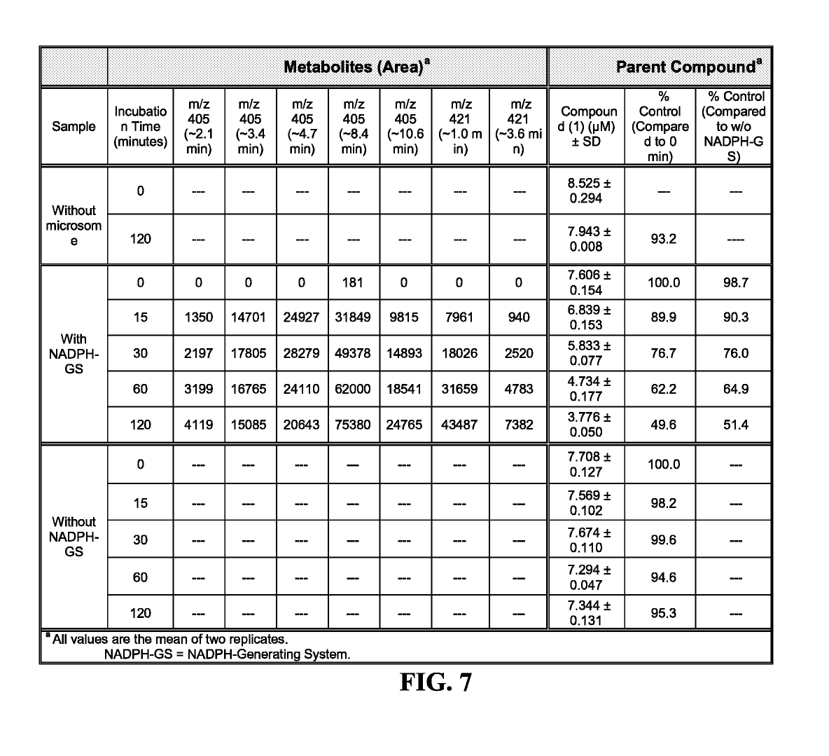

FIG. 8 is a table of Compound (1) concentration and its potential metabolites following incubations of 10 μM Compound (1) with 0.1 M phosphate buffer, 3 mM MgCl₂, 1 mM EDTA and pooled human liver microsomes in the presence and in the absence of NADPH-generating system at various timepoints.

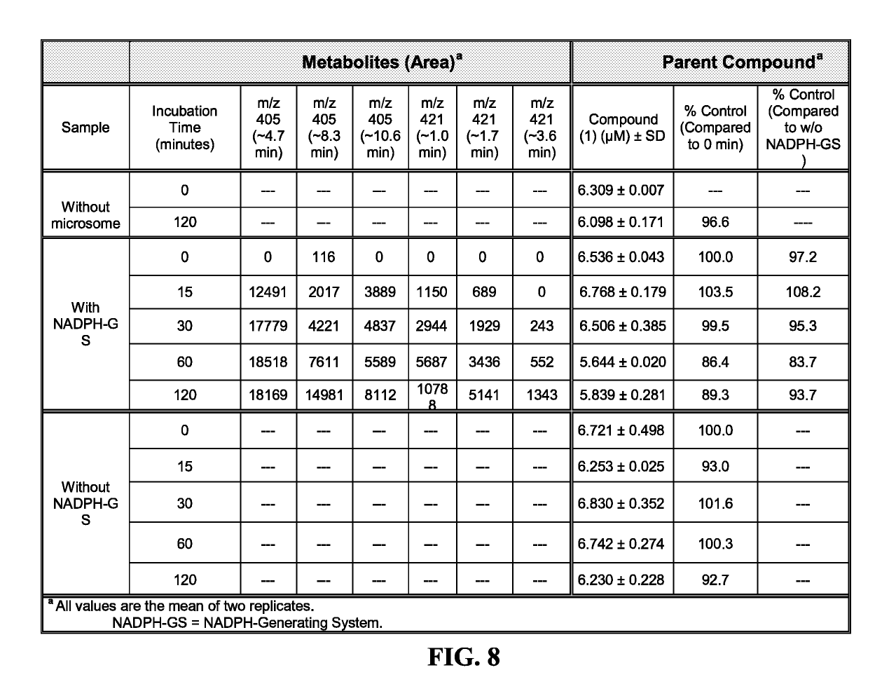

FIG. 9 is a representative chromatogram of Compound (1) (m/z 389).

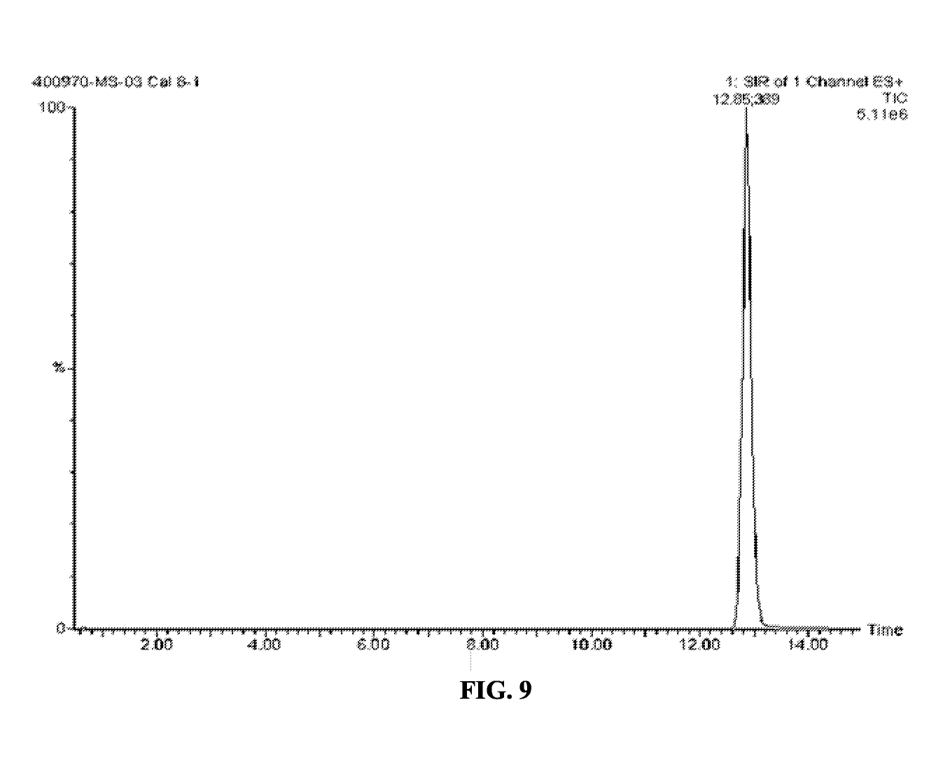

FIG. 10 is a representative chromatogram (m/z 405) of incubation of 10 μM Compound (1) in rat liver microsomes for up to 120 minutes.

FIG. 11 is a representative chromatogram (m/z 421) of incubation of 10 μM Compound (1) in rat liver microsomes for up to 120 minutes.

FIG. 12 is a representative chromatogram (m/z 405) of incubation of 10 μM Compound (1) in rat liver microsomes with EDTA and MgCl₂ for up to 120 minutes.

FIG. 13 is a representative chromatogram (m/z 421) of incubation of 10 μM Compound (1) in rat liver microsomes with EDTA and MgCl₂ for up to 120 minutes.

FIG. 14 is a representative chromatogram (m/z 405) of incubation of 10 μM Compound (1) in dog liver microsomes for up to 120 minutes.

FIG. 15 is a representative chromatogram (m/z 421) of incubation of 10 μM Compound (1) in dog liver microsomes for up to 120 minutes.

FIG. 16 is a representative chromatogram (SIR m/z 405) of incubation of 10 μM Compound (1) in dog liver microsomes with EDTA and MgCl₂ for up to 120 minutes.

FIG. 17 is a representative chromatogram (SIR m/z 421) of incubation of 10 μM Compound (1) in dog liver microsomes with EDTA and MgCl₂ for up to 120 minutes.

FIG. 18 is a representative chromatogram (SIR m/z 405) of incubation of 10 μM Compound (1) in monkey liver microsomes for up to 120 minutes.

FIG. 19 is a representative chromatogram (SIR m/z 421) of incubation of 10 μM Compound (1) in monkey liver microsomes for up to 120 minutes.

FIG. 20 is a representative chromatogram (SIR m/z 405) of incubation of 10 μM Compound (1) in monkey liver microsomes with EDTA and MgCl₂ for up to 120 minutes.

FIG. 21 is a representative chromatogram (SIR m/z 421) of incubation of 10 μM Compound (1) in monkey liver microsomes with EDTA and MgCl₂ for up to 120 minutes.

FIG. 22 is a representative chromatogram (SIR m/z 405) of incubation of 10 μM Compound (1) in human liver microsomes for up to 120 minutes.

FIG. 23 is a representative chromatogram (SIR m/z 421) of incubation of 10 μM Compound (1) in human liver microsomes for up to 120 minutes.

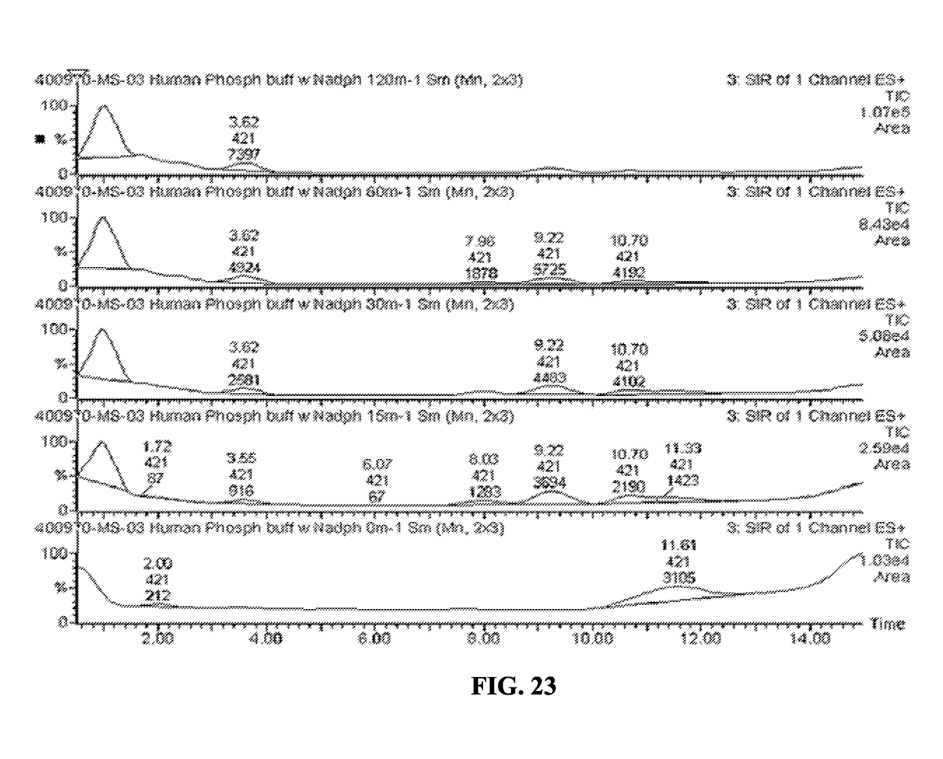

FIG. 24 is a representative chromatogram (SIR m/z 405) of incubation of 10 μM Compound (1) in human liver microsomes with EDTA and MgCl₂ for up to 120 minutes.

FIG. 25 is a representative chromatogram (SIR m/z 421) of incubation of 10 μM Compound (1) in human liver microsomes with EDTA and MgCl₂ for up to 120 minutes.

FIG. 26 is a chromatogram of the parent compound from dog plasma in 0.1% formic acid in water and 0.1% formic acid in acetonitrile.

FIG. 27 is a chromatogram of the parent compound from monkey plasma in 0.1% formic acid in water and 0.1% formic acid in acetonitrile.

FIG. 28 is a chromatogram of the parent compound from monkey plasma using the optimized HPLC parameters in 0.1% formic acid in water and 0.1% formic acid in acetonitrile.

FIG. 29 is a calibration curve of the parent compound in human plasma.

FIG. 30 is the fragmentation pattern of the parent compound on direct infusion.

FIG. 31 is the fragmentation pattern of 0.417 ng/mL parent in analytical method diluent.

FIG. 32 is the fragmentation pattern of 5 ng/mL of the parent in human plasma.

FIG. 33 is the fragmentation pattern of 10 ng/mL of the parent in human plasma.

FIG. 34 is a chromatogram illustrating MRM 389 to 195 in extracted cynomolgus monkey plasma.

FIG. 35 is a chromatogram illustrating MRM 389 to 195 of Std. 1.

FIG. 36 is a chromatogram illustrating MRM 389 to 195 of Std. 2.

FIG. 37 is a chromatogram illustrating MRM 389 to 195 of Std. 3.

FIG. 38 is a chromatogram illustrating MRM 389 to 195 of Std. 5.

FIG. 39 is a chromatogram illustrating MRM 389 to 195 of Std. 6.

FIG. 40 is a chromatogram illustrating MRM 389 to 195 of Std. 7.

FIG. 41 is a product ion spectrum of the parent using MS/MS parameters optimized for the parent.

FIG. 42 is a product ion spectrum of Std. 1 using MS/MS parameters optimized for the parent.

### DETAILED DESCRIPTION OF THE INVENTION

### Certain Chemical Terminology

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Definition of standard chemistry terms may be found in reference works, including Carey and Sundberg “ADVANCED ORGANIC CHEMISTRY 4^{TH} ED.” Vols. A (2000) and B (2001), Plenum Press, New York, hereby incorporated by reference in its entirety. Unless otherwise indicated, conventional methods of mass spectroscopy, NMR, HPLC, protein chemistry, biochemistry, recombinant DNA techniques and pharmacology, within the skill of the art are employed.

The term “alkenyl group,” as used herein, refers to a hydrocarbon chain having one or more double bonds therein. The double bond of an alkenyl group can be unconjugated or conjugated to another unsaturated group. Suitable alkenyl groups include, but are not limited to, (C₂-C₈)alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. The alkenyl moiety may be branched, straight chain, or cyclic (in which case, it would also be known as a “cycloalkenyl” group), and can be unsubstituted or substituted.

The term “alkoxy” as used herein, includes —O-(alkyl), where alkyl is as defined herein. By way of example only, C₁₋₆ alkoxy includes, but is not limited to, methoxy, ethoxy, and the like. An alkoxy group can be unsubstituted or substituted.

The term “alkyl,” as used herein, refers to a hydrocarbon group having from 1 to 10 carbon atoms and can include straight, branched, cyclic, saturated and/or unsaturated features. Whenever it appears herein, a numerical range such as “1 to 10” refers to each integer in the given range; e.g., “1 to 10 carbon atoms” or “C₁₋₁₀” or “(C₁-C₁₀)” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated. The alkyl moiety may be a “saturated alkyl” group, which means that it does not contain any alkene or alkyne moieties. Representative saturated alkyl groups include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, sec-butyl, t-butyl, n-pentyl, isopentyl, neopentyl, and n-hexyl, and longer alkyl groups, such as heptyl, and octyl. The alkyl moiety may also be an “unsaturated alkyl” moiety, which means that it contains at least one alkene or at least one alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. Representative unsaturated alkyl groups include, but are not limited to, ethenyl, propenyl, butenyl, propargyl and the like. An alkyl group can be unsubstituted or substituted. Substituted alkyl groups include, but are not limited to, halogen-substituted alkyl groups, such as, by way of example only, trifluoromethyl, pentafluoroethyl, and the like.

The term “alkynyl” group, as used herein, refers to a hydrocarbon chain having one or more triple bonds therein. The triple bond of an alkynyl group can be unconjugated or conjugated to another unsaturated group. Suitable alkynyl groups include, but are not limited to, (C₂-C₆)alkynyl groups, such as ethynyl, propynyl, butyryl, pentynyl, hexynyl, methylpropynyl, 4-methyl-1-butyryl, 4-propyl-2-pentynyl, and 4-butyl-2-hexynyl. The alkynyl moiety may be branched or straight chain, and can be unsubstituted or substituted.

The term “ester,” as used herein, refers to a chemical moiety with formula —COOR, where R is selected from the group consisting of alkyl, cycloalkyl, aryl, and heterocyclic (bonded through a ring carbon). Any hydroxy or carboxyl side chain on the compounds described herein can be esterified. The procedures and specific groups to make such esters are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety. An ester group can be unsubstituted or substituted.

The term “halogen,” as used herein, means fluoro, chloro, bromo or iodo. Preferred halogen groups are fluoro, chloro and bromo.

The term “heteroatom” as used herein means any atom in the periodic chart other than carbon or hydrogen. Such heteroatoms include, but are not limited to, halogens, such as fluorine, chlorine or bromine, chalcogens such as oxygen and sulfur, nitrogen, phosphorus, silicon, or boron. Preferred heteroatoms are fluorine, chlorine, oxygen, nitrogen, and sulfur.

The terms “haloalkyl,” “haloalkenyl,” “haloalkynyl” and “haloalkoxy” include alkyl, alkenyl, alkynyl and alkoxy structures that are substituted with one or more halogen groups or with combinations thereof.

The terms “glucuronide,” “glucuronido” and the like as used herein refers to a glucuronic acid linkage via a bond as exemplified below or at the 2, 3 or 4 hydroxy positions:

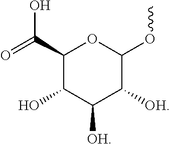

The terms “glucuronate,” “glucuronato” and the like as used herein refers to a glucuronic acid linkage via a bond as exemplified below:

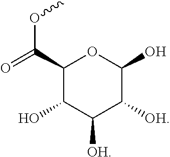

The term “membered ring,” as used herein, can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, imidazole, furan, and thiophene are 5-membered rings.

The term “moiety,” as used herein, refers to a specific segment or functional group of a molecule. Chemical moieties are often recognized chemical entities embedded in or appended to a molecule.

The term “protecting group,” as used herein, refers to a chemical moiety which blocks some or all reactive moieties and selectively prevents such groups from participating in chemical reactions until the protective group is removed.

The term “reactant,” as used herein, refers to a nucleophile or electrophile used to create covalent linkages.

Unless otherwise indicated, when a substituent is deemed to be “optionally substituted,” it is meant that the substituent is a group that may be substituted with one or more group(s) individually and independently selected from, for example, alkenyl, alkyl, alkoxy, alkylamine, alkylthio, alkynyl, amide, amino, including mono- and di-substituted amino groups, aryl, aryloxy, arylthio, carbonyl, carbocyclic, cyano, cycloalkyl, halogen, heteroalkyl, heteroalkenyl, heteroalkynyl, heteroaryl, heterocyclic, hydroxy, isocyanato, isothiocyanato, mercapto, nitro, O-carbamyl, N-carbamyl, O-thiocarbamyl, N-thiocarbamyl, C-amido, N-amido, S-sulfonamido, N-sulfonamido, C-carboxy, O-carboxy, perhaloalkyl, perfluoroalkyl, silyl, sulfonyl, thiocarbonyl, thiocyanato, trihalomethanesulfonyl, and the protected compounds thereof. The protecting groups that may form the protected compounds of the above substituents are known to those of skill in the art and may be found in references such as Greene and Wuts, Protective Groups in Organic Synthesis, 3^{rd} Ed., John Wiley & Sons, New York, N.Y., 1999, and Kocienski, Protective Groups, Thieme Verlag, New York, N.Y., 1994, which are incorporated herein by reference in their entirety.

### Certain Pharmaceutical Terminology

The term “acceptable” with respect to a formulation, composition or ingredient, as used herein, means having no persistent detrimental effect on the general health of the subject being treated.

The term “agonist,” as used herein, refers to a molecule such as a compound, a drug, an enzyme activator or a hormone modulator which enhances the activity of another molecule or the activity of a receptor site.

The term “antagonist,” as used herein, refers to a molecule such as a compound, a drug, an enzyme inhibitor, or a hormone modulator, which diminishes, or prevents the action of another molecule or the activity of a receptor site.

The term “carrier,” as used herein, refers to relatively nontoxic chemical compounds or agents that facilitate the incorporation of a compound into cells or tissues.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “kit” and “article of manufacture” are used as synonyms.

The term “metabolite,” as used herein, refers to a derivative of a compound which is formed when the compound is metabolized. Said metabolites may also be produced by discreet chemical synthesis, after determination of their structure through spectroscopic and other analytical means.

The term “active metabolite,” as used herein, refers to a biologically active derivative of a compound that is formed when the compound is metabolized.

The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996).

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator,” as used herein, refers to a molecule that interacts with a target either directly or indirectly. The interactions include, but are not limited to, the interactions of an agonist and an antagonist.

By “pharmaceutically acceptable,” as used herein, refers a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” of a compound, as used herein, refers to a salt that is pharmaceutically acceptable.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound of Formula (1) and a co-agent, are both administered to a patient simultaneously in the form of a single entity or dosage. The term “non-fixed combination” means that the active ingredients, e.g. a compound of Formula (1) and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “pharmaceutical composition,” as used herein, refers to a mixture of an active compound or compounds with other chemical components, such as carriers, stabilizers, diluents, dispersing agents, suspending agents, thickening agents, and/or excipients.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition.

### Illustrative Biological Activity

Androgen Receptor (AR)

Androgens bind to a specific receptor, the androgen receptor (AR), inside the cells of target tissues. The AR is expressed in numerous tissues of the body and is the receptor through which the physiological as well as the pathophysiological effects of endogenous androgen ligands, such as testosterone (T) and dihydrotestosterone (DHT), are expressed. Structurally, the AR is composed of three main functional domains: the ligand binding domain (LBD), the DNA-binding domain, and amino-terminal domain. A compound that binds to the AR and mimics the effects of an endogenous AR ligand is referred to as an AR agonist, whereas a compound that inhibits the effects of an endogenous AR ligand is termed an AR antagonist. Binding of androgen to the receptor activates it and causes it to bind to DNA binding sites adjacent to target genes. From there it interacts with coactivator proteins and basic transcription factors to regulate the expression of the gene. Thus, via its receptor, androgens cause changes in gene expression in cells. These changes ultimately have consequences on the metabolic output, differentiation or proliferation of the cell that are visible in the physiology of the target tissue. In the prostate, androgens stimulate the growth of prostate tissue and prostate cancer cells by binding to the AR that is present within the cytoplasm of androgen sensitive tissue.

Compounds which selectively modulate AR are of clinical importance in the treatment of or prevention of a variety of diseases and conditions, including, but not limited to, prostate cancer, benign prostatic hyperplasia, hirsutism in women, alopecia, anorexia nervosa, breast cancer, acne, musculoskeletal conditions, such as bone disease, hematopoietic conditions, neuromuscular disease, rheumatological disease, cancer, AIDS, cachexia, for hormone replacement therapy (HRT), employed in male contraception, for male performance enhancement, for male reproductive conditions, and primary or secondary male hypergonadism.

Castration Resistant Prostate Cancer

Agents that block the action (antiandrogens) of endogenous hormones (e.g., testosterone) are highly effective and routinely used for the treatment of prostate cancer (androgen ablation therapy). While initially effective at suppressing tumor growth, these androgen ablation therapies eventually fail in almost all patients, leading to “castration resistant prostate cancer” (“CRPC”). Most, but not all, prostate cancer cells initially respond to androgen withdrawal therapy. However, with time, surviving populations of prostate cancer cells emerge because they have responded to the selective pressure created by androgen ablation therapy and are now refractory to it. Not only is the primary cancer refractory to available therapies, but cancer cells may also break away from the primary tumor and travel in the bloodstream, spreading the disease to distant sites (especially bone). Among other effects, this causes significant pain and further bone fragility.

It is contemplated that CRPC cells survive in an environment characterized by low levels of circulating androgens by amplifying at least three different pathways to enhance the response to the intracellular androgens that remain available. These include: (1) Up-regulation of the expression of the AR, which increases AR copy number and hence the sensitivity of the cells to low levels of circulating androgen induced by medical castration therapy; (2) Increase in the expression of enzymes involved in the importation of androgens that remain in cells after androgen deprivation therapy; (3) Increase in the expression of genes that regulate steroidogenesis, permitting the CRPC cells to synthesize their own androgens. A critical enzyme in the steroidogenic pathway is cytochrome C_{17α}-hydroxylase/C₁₇₋₂₀-lyase (CYP17), the enzyme that controls androgen production in the adrenals, testes, and prostate.

Described herein, in certain embodiments, are compounds, methods of making such compounds, pharmaceutical compositions and medicaments comprising such compounds, and methods of using such compounds to treat androgen receptor mediated diseases or conditions including, but not limited to, prostate cancer, benign prostatic hyperplasia, hirsutism in women, alopecia, anorexia nervosa, breast cancer, acne, musculoskeletal conditions, such as bone disease, hematopoietic conditions, neuromuscular disease, rheumatological disease, cancer, AIDS, cachexia, for hormone replacement therapy (HRT), employed in male contraception, for male performance enhancement, for male reproductive conditions, and primary or secondary male hypogonadism. In some embodiments, the androgen receptor mediated disease or condition is prostate cancer. In certain embodiments, the prostate cancer is castration resistant prostate cancer.

Described herein, in certain embodiments, are compounds, pharmaceutical compositions, and medicaments comprising such compounds, and methods of using such compounds that decrease androgen biosynthesis, decrease androgen receptor signaling and decrease androgen receptor sensitivity.

In one aspect, the compounds, pharmaceutical compositions and medicaments comprising such compounds, and methods of using such compounds decrease androgen biosynthesis. In some embodiments, the compounds disclosed herein inhibit the activity of enzymes that controls androgen production. In certain embodiments, the compounds disclosed herein inhibit the activity of cytochrome C_{17α}-hydroxylase/C₁₇₋₂₀-lyase (CYP17).

In one aspect, the compounds, pharmaceutical compositions and medicaments comprising such compounds, and methods of using such compounds decrease androgen receptor signaling. In some embodiments, the compounds disclosed herein bind to the AR and are a competitive inhibitor of testosterone binding.

In one aspect, the compounds, pharmaceutical compositions and medicaments comprising such compounds, and methods of using such compounds decrease androgen receptor sensitivity. In some embodiments, the compounds disclosed herein reduce the content of AR protein within the cell and diminish the ability of the cell to be sustained by low levels of androgenic growth signals. In certain embodiments, the compounds disclosed herein are formed in vivo after administration of a drug to a subject.

### Compounds

Compounds of Formula (1), pharmaceutically acceptable salts, pharmaceutically acceptable N-oxides, pharmaceutically active metabolites, pharmaceutically acceptable prodrugs, pharmaceutically acceptable polymorphs and pharmaceutically acceptable solvates thereof, modulate the activity of steroid hormone nuclear receptors and, as such, are useful for treating androgen receptor mediated diseases or conditions.

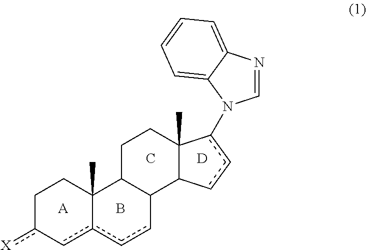

In compounds of Formula (1), the ABCD ring structure is the “A”, “B”, “C” and “D” ring portions of a steroid or an analog thereof, which are optionally substituted; X is glucuronido, glucuronato, O-linked sulfate, OH or O; and wherein the dashed lines can be taken at each occurrence independently to be double or single bonds, such to make a valence satisfied and stable molecule.

Optional substituents of the ABCD ring structure include one or more of: C₁-C₆-alkyl and halogenated C₁-C₆-alkyl; C₁-C₆-alkenyl and halogenated C₁-C₆-alkenyl, including where the double bond is directly attached to the ring structure; halogen; amino; aminoalkylene; hydroxyimino; n,n+1-epoxy; carbonyl (oxo); glucuronido, glucuronato, O-linked sulfate, and hydroxy. Hydrogen substituents on adjacent carbon atoms of the ABCD ring structure can be optionally removed and replaced by an additional bond between the adjacent carbon atoms to result in a double bond between these carbons in the ring structure. In some embodiments, optional substitutions on the ABCD ring structure are methyl groups at the 10 and/or 13 positions of the ring structure.

Certain embodiments of Formula (1) include two substituents, with each substituent chosen independently from a hydroxy, carbonyl (oxo), or n,n+1 epoxy at any position of the “A”, “B”, “C” and “D” ring. Certain embodiments of Formula (1) include one substituent chosen independently from a hydroxy, carbonyl (oxo), or n,n+1 epoxy at any position of the “A”, “B”, “C” and “D” ring.

Certain specific embodiments of Formula (1) are shown below as Formula (2), Formula (3), Formula (4), Formula (5), Formula (6), Formula (7), Formula (8), Formula (9), Formula (10), Formula (11), Formula (12), Formula (13), Formula (14), Formula (15), Formula (16), Formula (17), Formula (18), Formula (19), Formula (20), Formula (21), Formula (22), Formula (23) Formula (24), Formula (25), Formula (26), Formula (27), Formula (28), Formula (29), Formula (30), and Formula (31), wherein X is a glucuronido, glucuronato or O-linked sulfate.

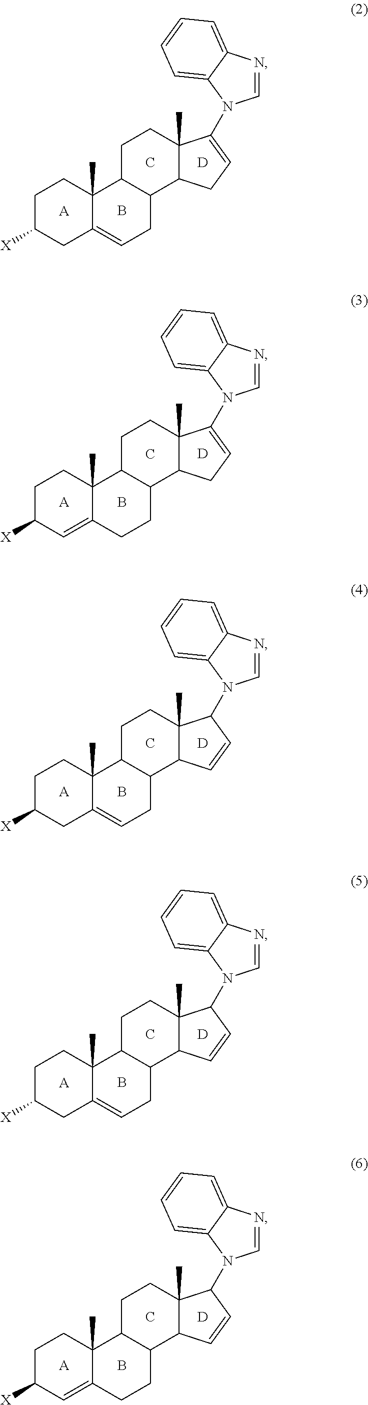

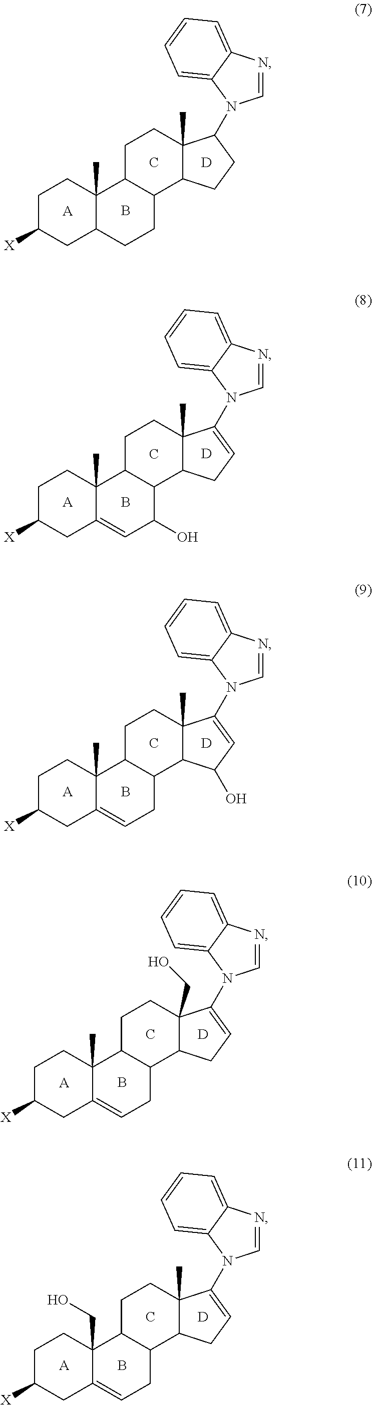

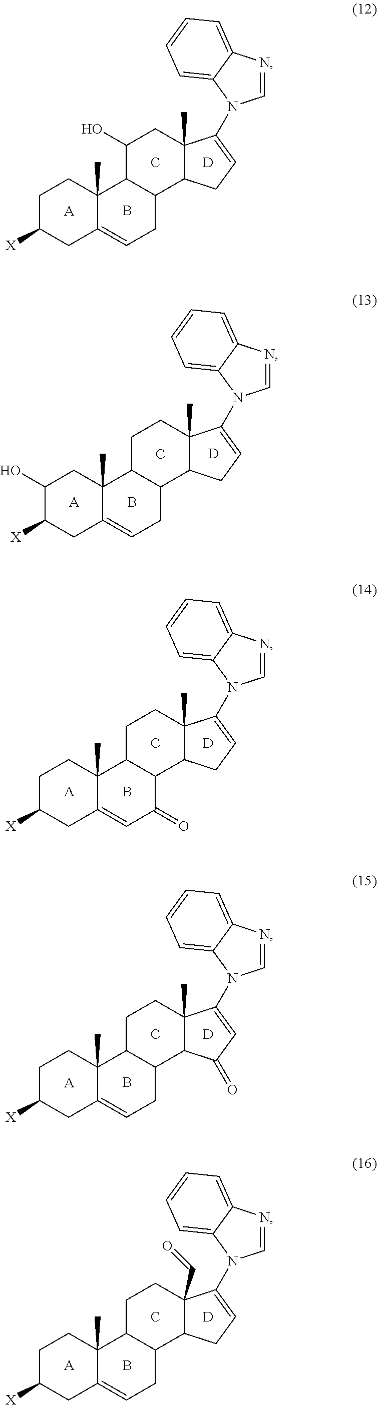

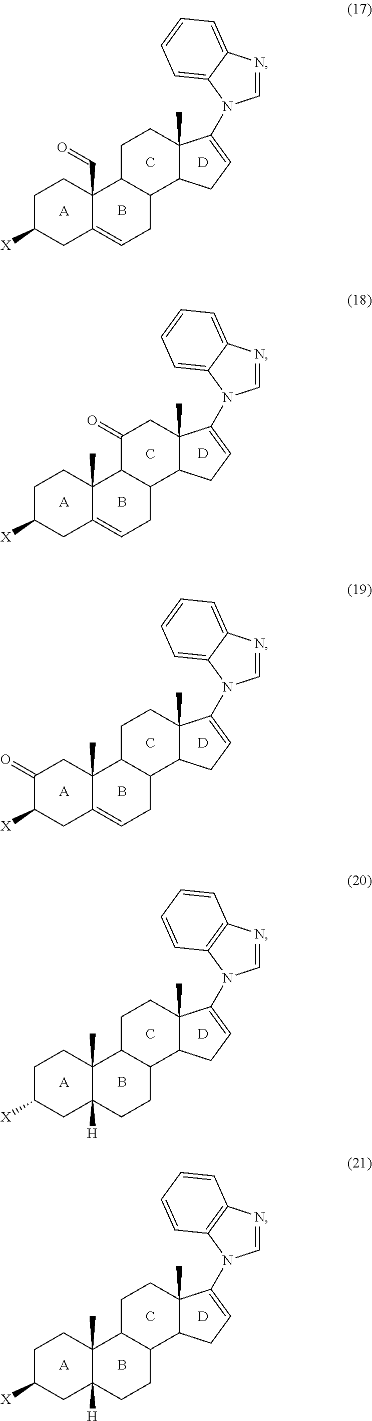

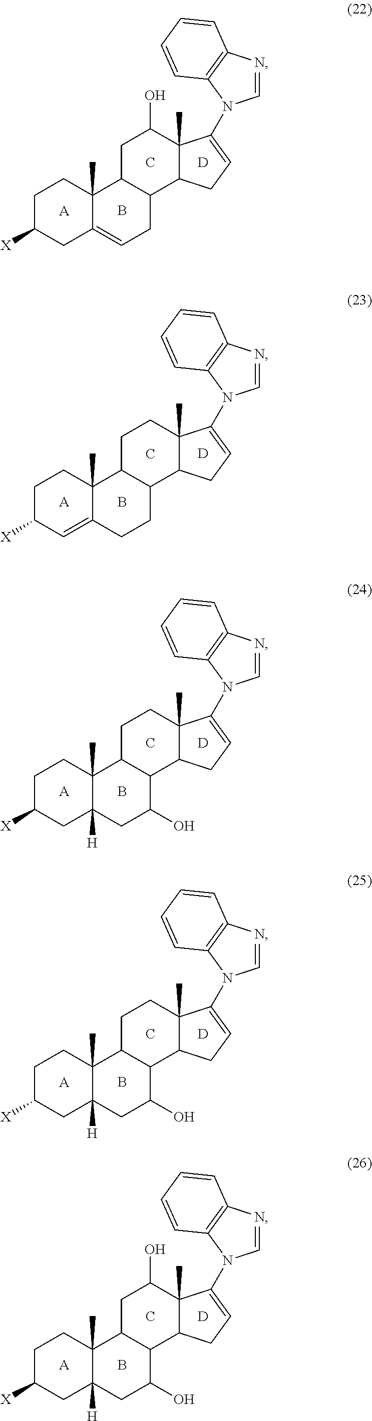

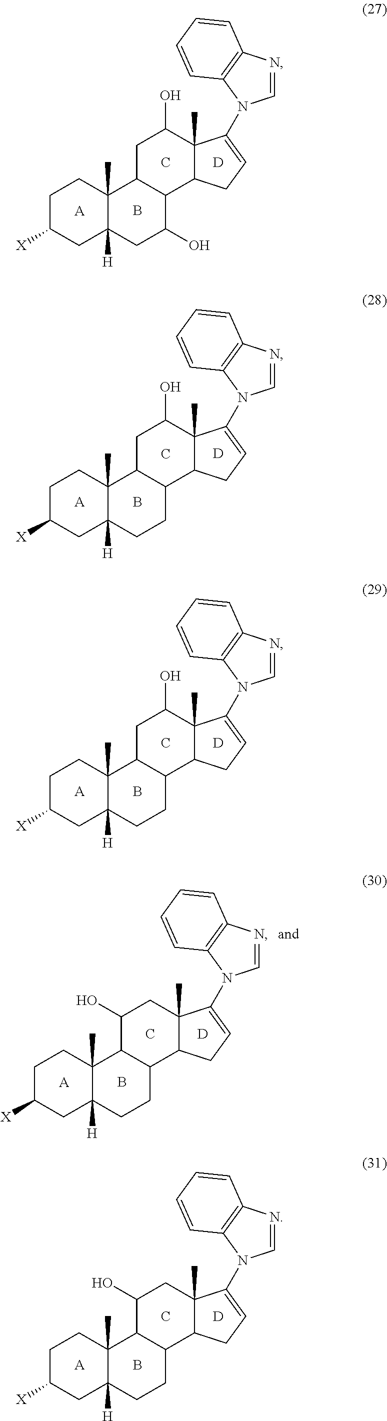

Certain specific embodiments of Formula (1) are shown below as Formula (32), Formula (33), Formula (34), Formula (35) Formula (36), Formula (37), Formula (38), Formula (39), Formula (40), Formula (41) Formula (42), Formula (43), Formula (44), Formula (45), Formula (46), Formula (47), Formula (48), Formula (49), Formula (50), Formula (51), Formula (52), Formula (53) Formula (54), Formula (55), Formula (56), Formula (57), Formula (58), Formula (59) Formula (60), Formula (61) Formula (62), Formula (63), Formula (64), and Formula (65).

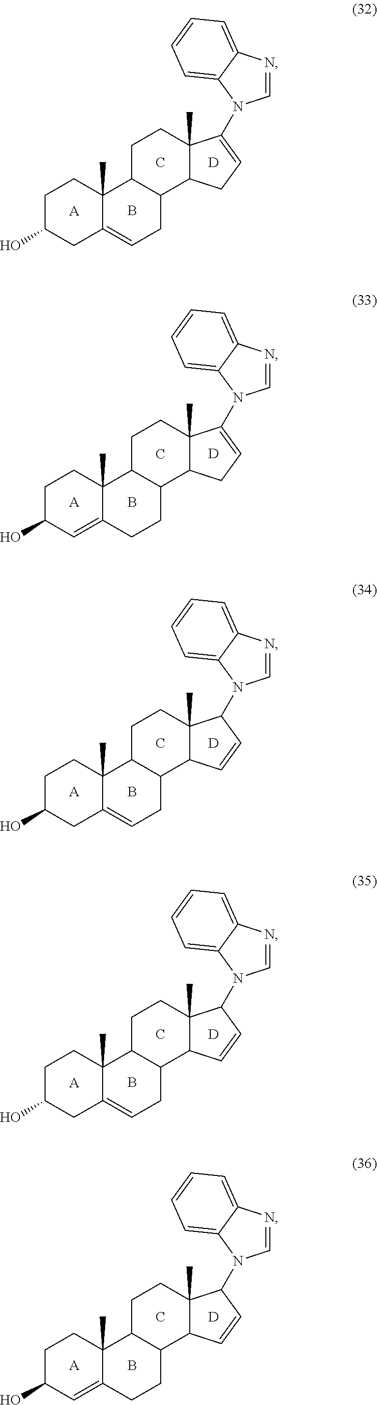

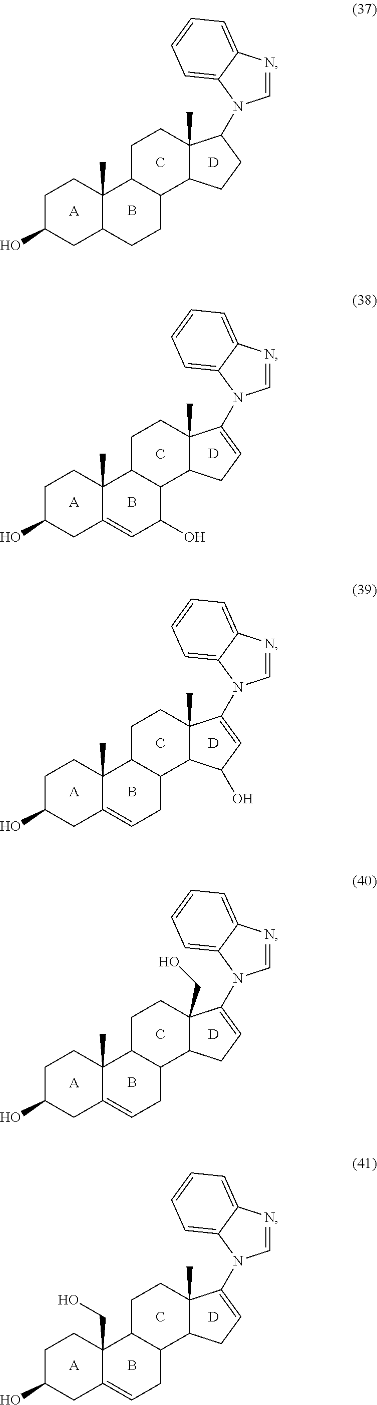

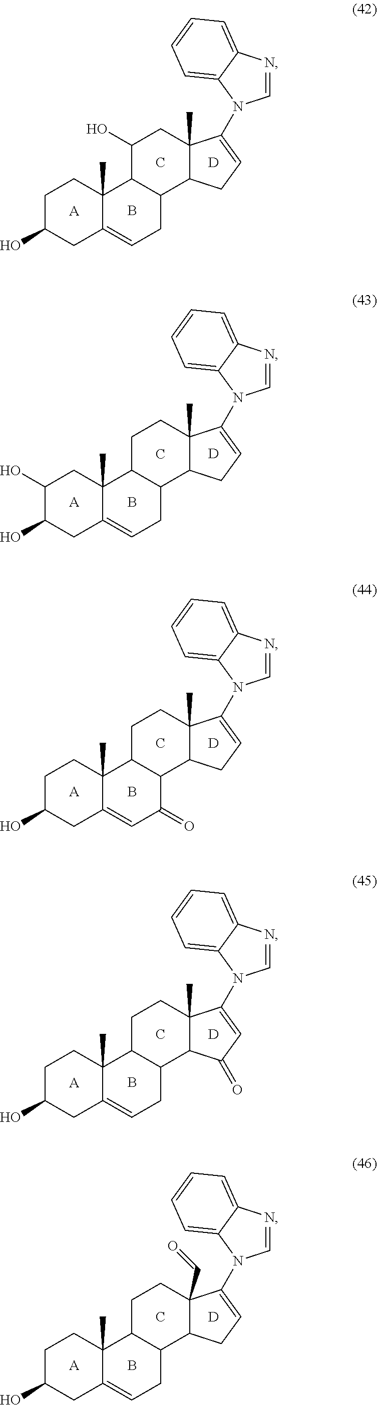

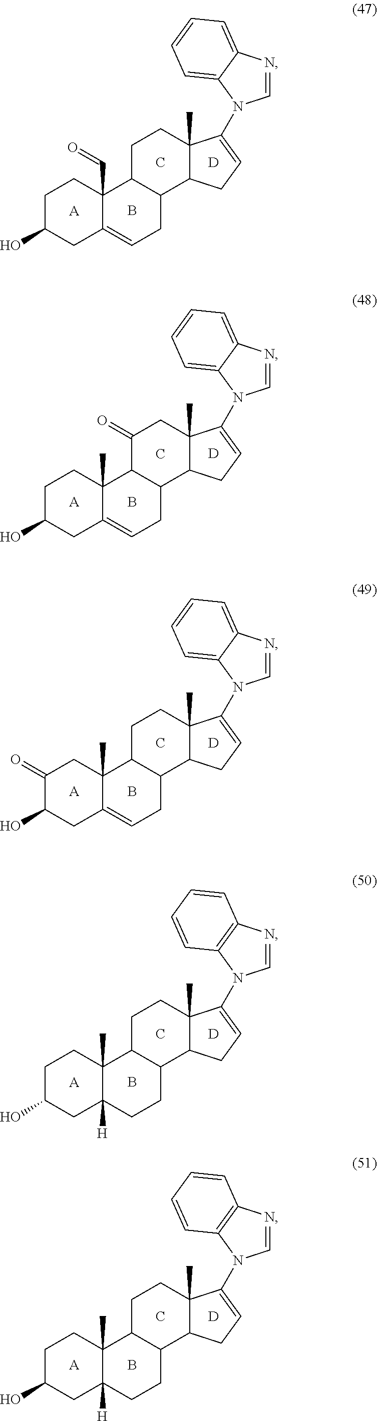

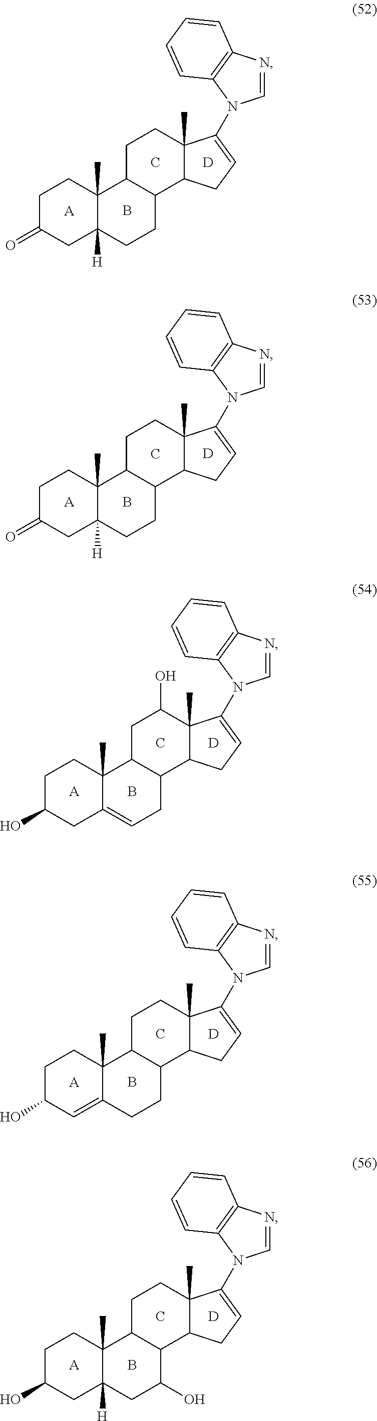

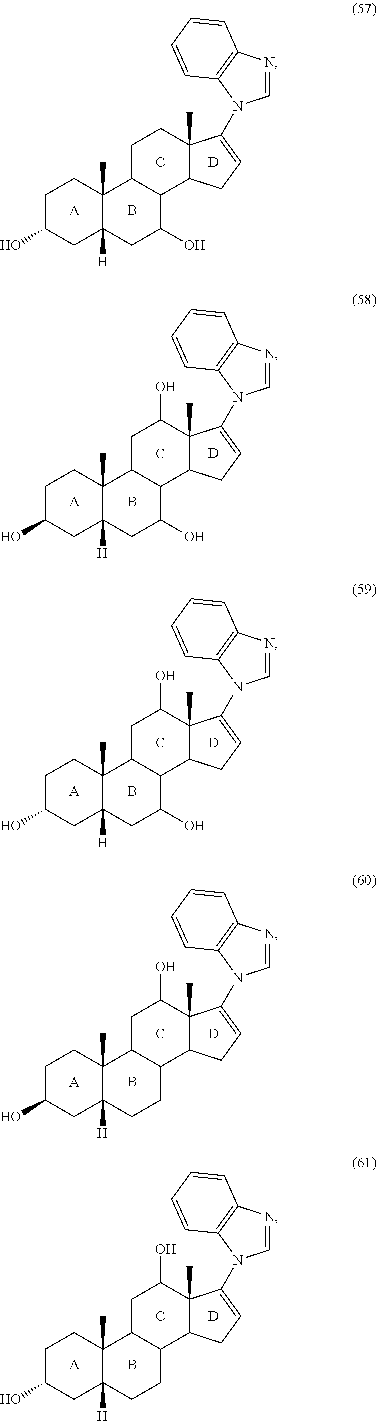

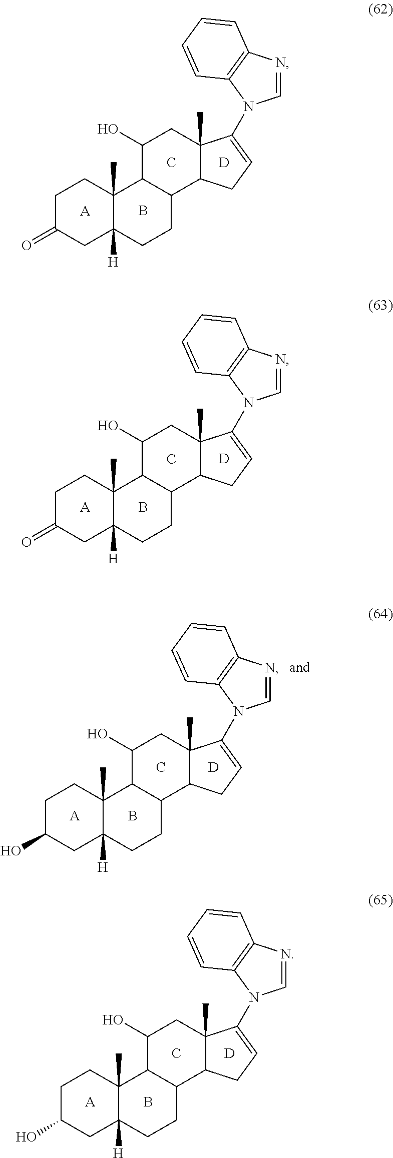

### Synthesis of the Compounds

Compounds of Formula (1) may be synthesized using standard synthetic techniques known to those of skill in the art or using methods known in the art in combination with methods described herein. In additions, solvents, temperatures and other reaction conditions presented herein may vary according to the practice and knowledge of those of skill in the art.

The starting material used for the synthesis of compounds of Formula (1) can be obtained from commercial sources, such as Aldrich Chemical Co. (Milwaukee, Wis.), Sigma Chemical Co. (St. Louis, Mo.), or the starting materials can be synthesized. The compounds described herein, and other related compounds having different substituents can be synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001), and Green and Wuts, PROTECTIVE GROUPS IN ORGANIC SYNTHESIS 3^{rd} Ed., (Wiley 1999) (all of which are incorporated by reference in their entirety). General methods for the preparation of compounds as disclosed herein may be derived from known reactions in the field, and the reactions may be modified by the use of appropriate reagents and conditions, as would be recognized by the skilled person, for the introduction of the various moieties found in the formulae as provided herein. As a guide the following synthetic methods may be utilized.

Compounds of Formula (1) can be prepared as a pharmaceutically acceptable salts formed when an acidic proton present in the parent compound either is replaced by a metal ion, for example an alkali metal ion, an alkaline earth ion, or an aluminum ion; or coordinates with an organic base. In addition, the salt forms of the disclosed compounds can be prepared using salts of the starting materials or intermediates.

Various methods of making Compound (1) are contemplated and the following descriptions are provided as non-limiting examples. In some embodiments, one or more of the following chemical reactions is performed in an inert atmosphere, for example, nitrogen or argon. In some embodiments, the temperature of the reaction is monitored. In some embodiments, the reaction is monitored by HPLC or TLC. In some embodiments, the pH of the reaction is monitored. In some embodiments, the temperature of the reaction is controlled. In some embodiments, the purity of the product is determined by HPLC. In some embodiments, the experiments are run on small scale, medium scale, large scale, analytical scale, or manufacturing scale. In some embodiments, the product is clarified by filtration through a pad comprising silica gel, celite or a combination thereof.

In some embodiments, the synthesis is performed on large scale. In some embodiments, large scale comprises a scale of about 1 to about 10 kg. In some embodiments, the synthesis is performed on manufacturing scale. In some embodiments, manufacturing scale comprises a scale of greater than about 10 kg. In some embodiments, manufacturing scale comprises a scale of about 10 to about 1,000 kg. In some embodiments, manufacturing scale comprises a scale of about 10 to about 100 kg. In some embodiments, manufacturing scale comprises a scale of about 10 to about 50 kg. In some embodiments, manufacturing scale comprises a scale of about 33.4 kg.

In some embodiments, an experiment is performed on a smaller scale to gather information to be used to plan or perform synthesis on a manufacturing scale. In some embodiments, the results obtained on the smaller scales are expected to be reproducible on manufacturing scale. In some embodiments, the results obtained on smaller scales are not expected to be reproducible on manufacturing scale. In some embodiments, the yields obtained on manufacturing scale are greater than the yields obtained on smaller scales. In some embodiments, the yields obtained on manufacturing scale are lesser than the yields obtained on smaller scales.

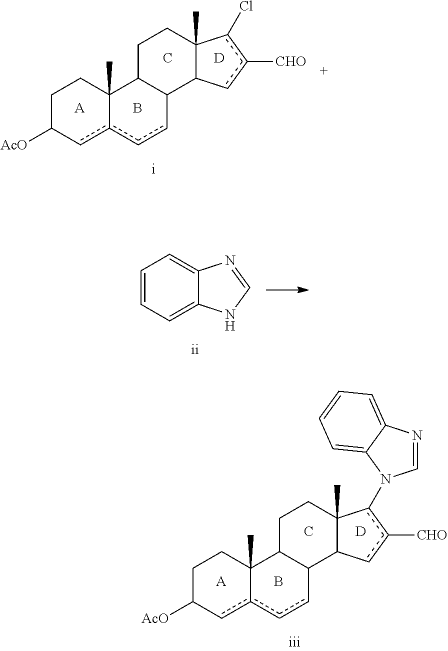

In one embodiment, a solution of a compound of Formula i in a solvent is prepared. A compound of Formula II is then contacted to the solution, and the resultant mixture is heated in the presence of a base for a period of time sufficient to provide a compound of Formula iii. In some embodiments, the period of time is about 1 hour, about 2 hours, about 4 hours, about 8 hours, about 12 hours, or about 24 hours. In some embodiments, the time is from about 1 hour to about 24 hours. In some embodiments, the base comprises lithium carbonate, sodium carbonate, potassium carbonate, sodium bicarbonate, a sodium phosphate, or a potassium phosphate. In some embodiments, the solvent comprises DMF. In some embodiments, the temperature is about 50° C., about 70° C., about 100° C., about 150° C., or a temperature effective to sustain reflux conditions. In some embodiments, the temperature is from about 50° C. to about 200° C. The compound of Formula iii can be isolated from the reaction mixture and purified by any method known to one of skill in the art. Such methods include, but are not limited to, pouring an aqueous mixture into the reaction mixture, thereby effecting the precipitation of compound iii as a solid. The isolated compound of Formula iii may optionally be purified by any method known to one of skill in the art. Such methods include, but are not limited to, trituration with water.

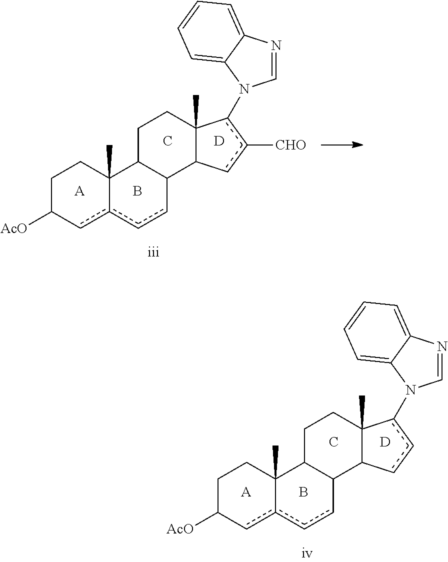

In one embodiment, a solution of a compound of Formula iii in a solvent is prepared, and the solution is contacted with a catalyst for a period of time sufficient to provide a compound of Formula iv. In some embodiments, the period of time is about 1 hour, about 2 hours, about 4 hours, about 8 hours, about 12 hours, or about 24 hours. In some embodiments, the time is from about 1 hour to about 24 hours. In some embodiments, the catalyst comprises palladium on carbon, platinum on carbon, a transition metal salt, or a transition metal complex. In some embodiments, the solvent comprises N-methylpyrrolidone. In some embodiments, the temperature is about 50° C., about 70° C., about 100° C., about 150° C., about 190° C., about 200° C. or a temperature effective to sustain reflux conditions. In some embodiments, the temperature is from about 50° C. to about 250° C. The compound of Formula iv can be isolated from the reaction mixture and purified by any method known to one of skill in the art. Such methods include, but are not limited to, in-line filtration and/or crystallization. The isolated compound of Formula iii may optionally be purified by any method known to one of skill in the art.

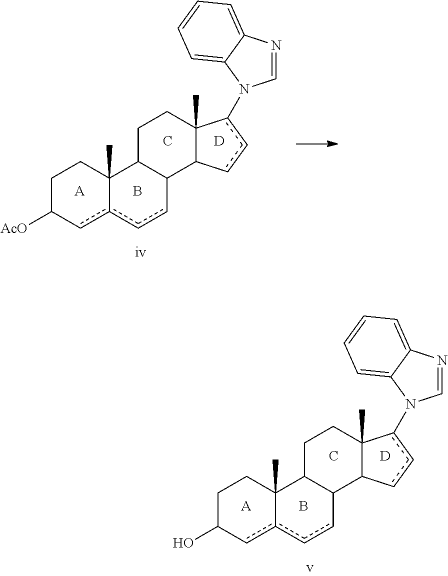

In one embodiment, a solution of a compound of Formula iv in a solvent is prepared, and the solution is contacted with a base for a period of time sufficient to provide a compound of Formula v (i.e., Compound (1)). In some embodiments, the period of time is about 1 hour, about 2 hours, about 4 hours, about 8 hours, about 12 hours, or about 24 hours. In some embodiments, the time is from about 1 hour to about 24 hours. In some embodiments, the base comprises lithium hydroxide, sodium hydroxide, potassium hydroxide, sodium methoxide, potassium methoxide, sodium ethoxide, potassium ethoxide, mixed alkali alkoxides (e.g. lithium-potassium alkoxides), lithium carbonate, sodium carbonate, potassium carbonate, sodium bicarbonate, a sodium phosphate, or a potassium phosphate. In some embodiments, the solvent comprises water, methanol, ethanol, 2-propanol, t-butanol, or mixtures thereof. In some embodiments, the solvent comprises methanol and the base comprises sodium methoxide. In some embodiments, the temperature is about 35° C., about 50° C., about 70° C., about 100° C., or a temperature effective to sustain reflux conditions. In some embodiments, the temperature is from about 25° C. to about 100° C. The compound of Formula v can be isolated from the reaction mixture and purified by any method known to one of skill in the art. Such methods include, but are not limited to, extraction. The isolated compound of Formula iii may optionally be purified by any method known to one of skill in the art. Such methods include, but are not limited to, trituration.

### Further Forms of Compounds

For convenience, the form and other characteristics of the compounds described in this section and other parts herein use a single formula, such as “Formula (1),” by way of example. However, the form and other characteristics of the compounds described herein apply equally well to all formulas presented herein that fall within the scope of Formula (1). For example, the form and other characteristics of the compounds described herein can be applied to compounds having the structure of Formula (2), Formula (3), Formula (4), Formula (5), Formula (6), Formula (7), Formula (8), Formula (9), Formula (10), Formula (11), Formula (12), Formula (13), Formula (14), Formula (15), Formula (16), Formula (17), Formula (18), Formula (19), Formula (20), Formula (21), Formula (22), Formula (23) Formula (24), Formula (25), Formula (26), Formula (27), Formula (28), Formula (29), Formula (30), Formula (31), Formula (32), Formula (33), Formula (34), Formula (35), Formula (36), and Formula (37) as well as to all of the specific compounds that fall within the scope of these generic formula.

Compounds of Formula (1) can be prepared as a pharmaceutically acceptable acid addition salt (which is a type of a pharmaceutically acceptable salt) by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid. Said salts can also be generated by salt exchange either from solution by precipitation adding the desired counterion, or by using an appropriate media, such as an ion exchange resin. Such methods can be used to form salts including, but not limited to tetraphenylborate, tetrafluoroborate and hexafluorophosphate.

Alternatively, compounds of Formula (1) can be prepared as a pharmaceutically acceptable base addition salts (which is a type of a pharmaceutically acceptable salt) by reacting the free acid form of the compound with a pharmaceutically acceptable inorganic or organic base, including, but not limited to organic bases such as ethanolamine, diethanolamine, triethanolamine, tromethamine, N-methylglucamine, and the like and inorganic bases such as aluminum hydroxide, calcium hydroxide, potassium hydroxide, sodium carbonate, sodium hydroxide, and the like.

It should be understood that a reference to a pharmaceutically acceptable salt includes the solvent addition forms or crystal forms thereof, particularly solvates or polymorphs. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds of Formula (1) can be conveniently prepared or formed during the processes described herein. By way of example only, hydrates of compounds of Formula (1) can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

Compounds of Formula (1) include crystalline forms, also known as polymorphs. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

Compounds of Formula (1) in unoxidized form can be prepared from N-oxides of compounds of Formula (1) by treating with a reducing agent, such as, but not limited to, sulfur, sulfur dioxide, triphenyl phosphine, trialkyl phosphine, lithium borohydride, sodium borohydride, sodium cyano borohydride, phosphorus trichloride, phosphorus tribromide, or the like in a suitable inert organic solvent, such as, but not limited to, acetonitrile, ethanol, aqueous dioxane, or the like at 0 to 80° C. Additionally said reducing agents can be covalently bound or coordinatively supported on a solid support, such as a resin or silica.

Compounds of Formula (1) described herein may be labeled isotopically (e.g. with a radioisotope) or by another other means, including, but not limited to, the use of chromophores or fluorescent moieties, bioluminescent labels, or chemiluminescent labels. Compounds of Formula (1) may possess one or more chiral centers and each center may exist in the R or S configuration. The compounds presented herein include all diastereomeric, enantiomeric, and epimeric forms as well as the appropriate mixtures thereof. Compounds of Formula (1) can be prepared as their individual diastereomers or epimers by reacting an epimeric mixture of the compound with an optically active resolving agent to form a mixture of chemically different compounds, separating the components, removing the resolving agent and recovering the pure epimers. While resolution of epimers can be carried out using covalent diastereomeric derivatives of the compounds described herein, dissociable complexes are preferred (e.g., crystalline diastereomeric salts). Diastereomers have distinct physical properties (e.g., melting point, boiling point, solubility, reactivity, etc.) and can be readily separated by taking advantage of these dissimilarities. The diastereomers can be separated by chiral chromatography, or preferably, by separation/resolution techniques based upon differences in solubility. The pure epimers are then recovered, along with the resolving agent, by any practical means that would not result in epimerization. A more detailed description of the techniques applicable to the resolution of stereoisomers of compounds from their stereoisomeric mixtures can be found in Jean Jacques, Andre Collet, Samuel H. Wilen, “Enantiomers, Racemates and Resolutions,” John Wiley And Sons, Inc., 1981, herein incorporated by reference in its entirety.

Additionally, the compounds and methods provided herein may exist as geometric isomers. The compounds and methods provided herein include all cis, trans, syn, anti, entgegen (E), and zusammen (Z) isomers as well as the appropriate mixtures thereof. In some situations, compounds may exist as tautomers. All tautomers are included within the formulas described herein are provided by compounds and methods herein. In additional embodiments of the compounds and methods provided herein, mixtures of enantiomers and/or diastereoisomers, resulting from a single preparative step, combination, or interconversion may also be useful for the applications described herein.

### Pharmaceutical Composition/Formulation

A pharmaceutical composition, as used herein, refers to a mixture of at compound(s) of Formula (1) with other chemical components, such as carriers, binders, stabilizers, diluents, dispersing agents, suspending agents, thickening agents, and/or excipients. The pharmaceutical composition facilitates administration of the compound to an organism. Pharmaceutical composition containing compound(s) of Formula (1) can be administered in therapeutically effective amounts as pharmaceutical compositions by any conventional form and route known in the art including, but not limited to: intravenous, oral, rectal, aerosol, parenteral, ophthalmic, pulmonary, transdermal, vaginal, optic, nasal, and topical administration.

One may administer the compound in a local rather than systemic manner, for example, via injection of the compound directly into an organ, often in a depot or sustained release formulation. Furthermore, one may administer pharmaceutical composition containing compound(s) of Formula (1) in a targeted drug delivery system, for example, in a liposome coated with organ-specific antibody. The liposomes will be targeted to and taken up selectively by the organ. In addition, the pharmaceutical composition containing compound(s) of Formula (1) may be provided in the form of a rapid release formulation, in the form of an extended release formulation, or in the form of an intermediate release formulation.

For oral administration, compound(s) of Formula (1) can be formulated readily by combining the active compounds with pharmaceutically acceptable carriers or excipients well known in the art. Such carriers enable the compounds described herein to be formulated as tablets, powders, pills, dragees, capsules, liquids, gels, syrups, elixirs, slurries, suspensions and the like, for oral ingestion by a patient to be treated.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores.

Dragee cores are provided with suitable coatings. For this purpose, concentrated sugar solutions may be used, which may optionally contain binding, disintegrating, flavoring or stabilizing agents; gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. In some embodiments, the capsule comprises a hard gelatin capsule comprising one or more of pharmaceutical, bovine, and plant gelatins. In certain instances, a gelatin is alkaline processed. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal or sublingual administration, the compositions may take the form of tablets, lozenges, or gels formulated in conventional manner. Parental injections may involve for bolus injection or continuous infusion. The pharmaceutical composition of compound(s) of Formula (1) may be in a form suitable for parenteral injection as a sterile suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

Compound(s) of Formula (1) can be administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

Formulations suitable for transdermal administration of compounds having the structure of Formula (1) may employ transdermal delivery devices and transdermal delivery patches and can be lipophilic emulsions or buffered, aqueous solutions, dissolved and/or dispersed in a polymer or an adhesive. Such patches may be constructed for continuous, pulsatile, or on demand delivery of pharmaceutical agents. Still further, transdermal delivery of compound(s) of Formula (1) can be accomplished by means of iontophoretic patches and the like. Additionally, transdermal patches can provide controlled delivery of compound(s) of Formula (1). The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

For administration by inhalation, compound(s) of Formula (1) may be in a form as an aerosol, a mist or a powder. Pharmaceutical compositions of Formula (1) are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, such as, by way of example only, gelatin for use in an inhaler or insufflator may be formulated containing a powder mix of the compound and a suitable powder base such as lactose or starch.

Compound(s) of Formula (1) may also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In practicing the methods of treatment or use provided herein, therapeutically effective amounts of compound(s) of Formula (1) provided herein are administered in a pharmaceutical composition to a mammal having a disease or condition to be treated. Preferably, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. Pharmaceutical compositions comprising a compound of Formula (1) may be manufactured in a conventional manner, such as, by way of example only, by means of conventional mixing, dissolving, granulating, dragee-making, levigating, emulsifying, encapsulating, entrapping or compression processes.

The pharmaceutical compositions will include at least one pharmaceutically acceptable carrier, diluent or excipient and a compound of Formula (1) described herein as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, compounds may exist as tautomers. All tautomers are included within the scope of the compounds presented herein. Additionally, the compounds described herein can exist in unsolvated as well as solvated forms with pharmaceutically acceptable solvents such as water, ethanol, and the like. The solvated forms of the compounds presented herein are also considered to be disclosed herein. In addition, the pharmaceutical compositions may include other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions can also contain other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The compositions may be in liquid solutions or suspensions, solid forms suitable for solution or suspension in a liquid prior to use, or as emulsions. These compositions may also contain minor amounts of nontoxic, auxiliary substances, such as wetting or emulsifying agents, pH buffering agents, and so forth.

A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

### Methods of Administration and Treatment Methods

Compounds of Formula (1) can be used in the preparation of medicaments for the treatment of diseases or conditions in which steroid hormone nuclear receptor activity contributes to the pathology and/or symptoms of the disease. In addition, a method for treating any of the diseases or conditions described herein in a subject in need of such treatment, involves administration of pharmaceutical compositions containing at least one compound of Formula (1), or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician. It is considered well within the skill of the art for one to determine such therapeutically effective amounts by routine experimentation (including, but not limited to, a dose escalation clinical trial).

Compositions containing the compound(s) described herein can be used to treat a disease-state or condition selected from: primary and secondary hyperaldosteronism, increased sodium retention, increased magnesium and potassium excretion (diuresis), increased water retention, hypertension (isolated systolic and combined systolic/diastolic), inflammation, malignancies such as leukemias and lymphomas, Cushing's congenital adrenal hyperplasia, chronic primary adrenal insufficiency, secondary adrenal insufficiency, prostate cancer, benign prostatic hyperplasia, alopecia, breast cancer, AIDS, cachexia, for hormone replacement therapy (HRT), employed in male contraception, testicular cancer, ovarian cancer, lung cancer, osteoporosis, bone loss, abnormally increased bone turnover, metastatic bone disease, and hypercalcemia of malignancyin a patient in need of such treatment, the method comprising administering to the patient an effective amount of a compound described herein, or a tautomer, prodrug, solvate, or salt thereof. In some embodiments, the compositions containing the compound(s) described herein can be used to treat prostate cancer. In other embodiments, the compositions containing the compound(s) described herein can be used to treat castration resistant prostate cancer.

Compositions containing the compound(s) described herein can be used to treat a disease-state or condition selected from: bladder cancer, prostate disease, prostatism, prostatalgia, prostatitis, prostatic hyperplasia, urinary incontinence, prostate neoplasms and cancers, penile neoplasms and cancers, testicular neoplasms and cancers, Sertoli-Leydig cell tumors, Leydig cell tumors, Sertoli cell tumors, Wilms tumors, renal cell carcinoma, nephroma, ureteral neoplasms, androgenic alopecia, hypogonadism, hyperpilosity, benign prostate hypertrophia, adenomas and neoplasias of the prostate (such as advanced metastatic prostate cancer), treatment of benign or malignant tumor cells containing the androgen receptor such as is the case for breast, brain, skin, ovarian, bladder, lymphatic, liver and kidney cancers, pancreatic cancers, osteoporosis, suppressing spermatogenesis, libido, cachexia, endometriosis, polycystic ovary syndrome, anorexia, androgen dependent age-related diseases and conditions, such as androgen supplement for age-related decreased testosterone levels in men, male menopause, male hormone replacement, male and female sexual dysfunction, and inhibition of muscular atrophy in ambulatory patients. In some embodiments, the compositions containing the compound(s) described herein can be used to treat prostate cancer. In other embodiments, the compositions containing the compound(s) described herein can be used to treat castration resistant prostate cancer.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition. In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disease or condition is retained. Patients can, however, require intermittent treatment on a long-term basis upon any recurrence of symptoms.

In certain instances, it may be appropriate to administer therapeutically effective amounts of at least one of the compounds described herein (or a pharmaceutically acceptable salts, pharmaceutically acceptable N-oxides, pharmaceutically active metabolites, pharmaceutically acceptable prodrugs, and pharmaceutically acceptable solvates thereof) in combination with another therapeutic agent. By way of example only, if one of the side effects experienced by a patient upon receiving one of the compounds herein is inflammation, then it may be appropriate to administer an anti-inflammatory agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. In any case, regardless of the disease or condition being treated, the overall benefit experienced by the patient may simply be additive of the two therapeutic agents or the patient may experience a synergistic benefit. For example, synergistic effects can occur with Compounds of Formula (1) and other substances used in the treatment of hypokalemia, hypertension, congestive heart failure, renal failure, in particular chronic renal failure, restenosis, atherosclerosis, syndrome X, obesity, nephropathy, post-myocardial infarction, coronary heart disease, increased formation of collagen, fibrosis and remodeling following hypertension and endothelial dysfunction. Examples of such compounds include anti-obesity agents, such as orlistat, anti-hypertensive agents, inotropic agents and hypolipidemic agents including, but not limited to, loop diuretics, such as ethacrynic acid, furosemide and torsemide; angiotensin converting enzyme (ACE) inhibitors, such as benazepril, captopril, enalapril, fosinopril, lisinopril, moexipril, perinodopril, quinapril, ramipril and trandolepril; inhibitors of the Na—K-ATPase membrane pump, such as digoxin; neutralendopeptidase (NEP) inhibitors; ACE/NEP inhibitors, such as omapatrilat, sampatrilat, and fasidotril; angiotensin II antagonists, such as candesartan, eprosartan, irbesartan, losartan, telmisartan and valsartan, in particularvalsartan; β-adrenergic receptor blockers, such as acebutolol, betaxolol, bisoprolol, metoprolol, nadolol, propanolol, sotalol and timolol; inotropic agents, such as digoxin, dobutamine and milrinone; calcium channel blockers, such as amlodipine, bepridil, diltiazem, felodipine, nicardipine, nimodipine, nifedipine, nisoldipine and verapamil; and 3-hydroxy-3-methyl-glutaryl coenzyme A reductase (HMG-CoA) inhibitors, such as lovastatin, pitavastatin, simvastatin, pravastatin, cerivastatin, mevastatin, velostatin, fluvastatin, dalvastatin, atorvastatin, rosuvastatin and rivastatin. Where the compounds described herein are administered in conjunction with other therapies, dosages of the co-administered compounds will of course vary depending on the type of co-drug employed, on the specific drug employed, on the disease or condition being treated and so forth. In addition, when co-administered with one or more biologically active agents, the compound provided herein may be administered either simultaneously with the biologically active agent(s), or sequentially. If administered sequentially, the attending physician will decide on the appropriate sequence of administering protein in combination with the biologically active agent(s).

In any case, the multiple therapeutic agents (one of which is one of the compounds described herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents. Multiple therapeutic combinations are envisioned.

In addition, compounds of Formula (1) may also be used in combination with procedures that may provide additional or synergistic benefit to the patient. By way of example only, patients are expected to find therapeutic and/or prophylactic benefit in the methods described herein, wherein pharmaceutical composition of Formula (1) and/or combinations with other therapeutics are combined with genetic testing to determine whether that individual is a carrier of a mutant gene that is known to be correlated with certain diseases or conditions.

Compounds of Formula (1) and combination therapies can be administered before, during or after the occurrence of a disease or condition, and the timing of administering the composition containing a compound can vary. Thus, for example, the compounds can be used as a prophylactic and can be administered continuously to subjects with a propensity to conditions or diseases in order to prevent the occurrence of the disease or condition. The compounds and compositions can be administered to a subject during or as soon as possible after the onset of the symptoms. The administration of the compounds can be initiated within the first 48 hours of the onset of the symptoms, preferably within the first 48 hours of the onset of the symptoms, more preferably within the first 6 hours of the onset of the symptoms, and most preferably within 3 hours of the onset of the symptoms. The initial administration can be via any route practical, such as, for example, an intravenous injection, a bolus injection, infusion over 5 minutes to about 5 hours, a pill, a capsule, transdermal patch, buccal delivery, and the like, or combination thereof. A compound is preferably administered as soon as is practicable after the onset of a disease or condition is detected or suspected, and for a length of time necessary for the treatment of the disease, such as, for example, from about 1 month to about 3 months. The length of treatment can vary for each subject, and the length can be determined using the known criteria. For example, the compound or a formulation containing the compound can be administered for at least 2 weeks, preferably about 1 month to about 3 years, and more preferably from about 1 month to about 10 years.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

The daily dosages appropriate for Compounds of Formula (1) described herein are from about 0.03 to about 60 mg/kg per body weight. An indicated daily dosage in the larger mammal, including, but not limited to, humans, is in the range from about 1 mg to about 4000 mg, conveniently administered in divided doses, including, but not limited to, up to four times a day or in retard form. Suitable unit dosage forms for oral administration comprise from about 1 mg to about 4000 mg active ingredient. In some embodiments, a single dose of compounds of Formula (1) is within the range of about 50 mg to about 2,000 mg. In some embodiments, a single dose of compounds of Formula (1) is about 90 mg, about 200 mg, about 250 mg, about 325 mg, about 650 mg, about 975 mg, about 1300 mg, about 1625 mg, or about 1950 mg. In some embodiments, an administration of compounds of Formula (1) of about 90 mg, about 325 mg, about 650 mg, about 975 mg, about 1300 mg, about 1625 mg, or about 1950 mg is given as multiple doses.

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, for determining the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in humans. The dosage of such compounds lies preferably within a range of circulating concentrations that include the ED₅₀ with minimal toxicity. The dosage may vary within this range depending upon the dosage form employed and the route of administration utilized.

### Incubation Studies

Incubation medium was optimized for the solubility of Compound (1) in the presence and in the absence of MgCl₂/EDTA. The dissolution of the test article appeared to be slightly better in the absence of MgCl₂/EDTA than in their presence. However, the role of the divalent cation, MgCl₂ in the activation of the CYP 450 activity is not well defined (Saito et al., 2006, Tamura et al., 1988). Therefore, the metabolic stability of Compound (1) was performed in the presence and in the absence of EDTA and MgCl₂.

### Standards and Quality Controls

The coefficient of determination (r²) value of linear regression for the standard curve was ≧0.998. Relative error (%) based on the back-calculated concentration in curve was within ±15% for all standards. Relative error (%) was within ±16% for all QCs.

### Negative Control

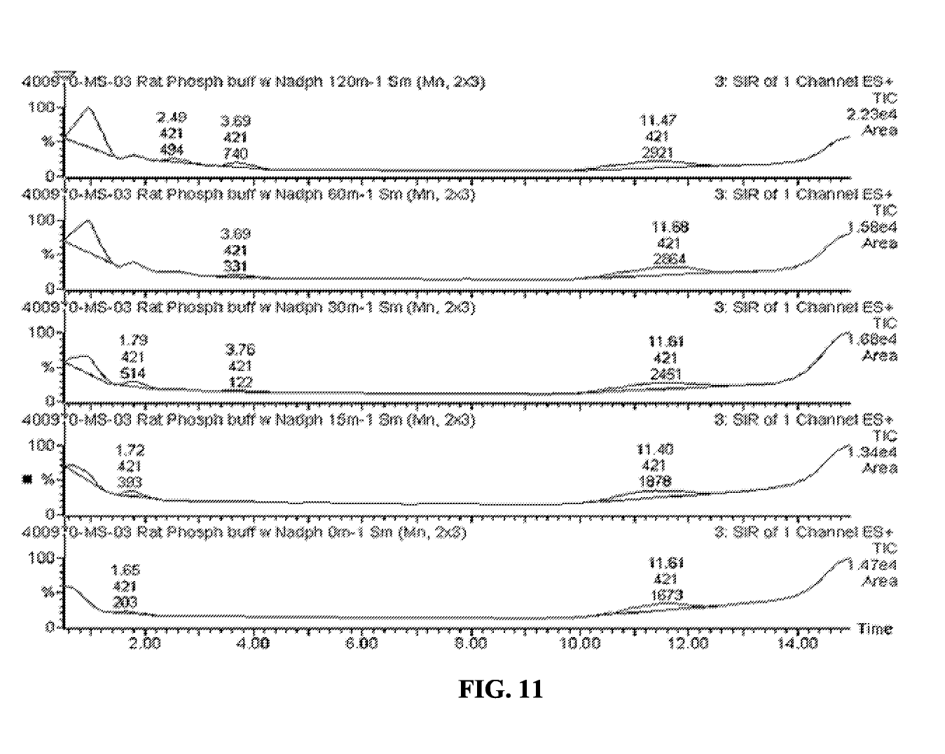

Two blank samples (0 and 120 minutes), containing Compound (1) but without microsomes (volume replaced by 0.1 M phosphate buffer), was included for each assay as a negative control. On all occasions, Compound (1) was chemically stable over the incubation period as demonstrated by the % recovery following 120 minutes close to 100% (within 7%) compared to the 0 minute (FIGS. 11-18).

### Metabolic Stability of Compound (1) in 0.1 M Phosphate Buffer

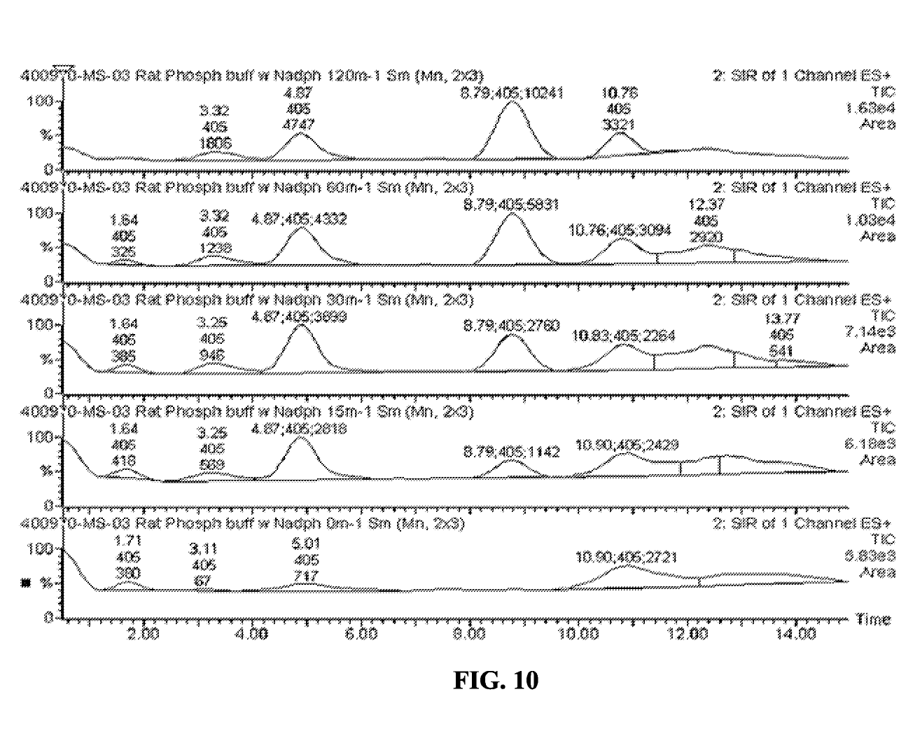

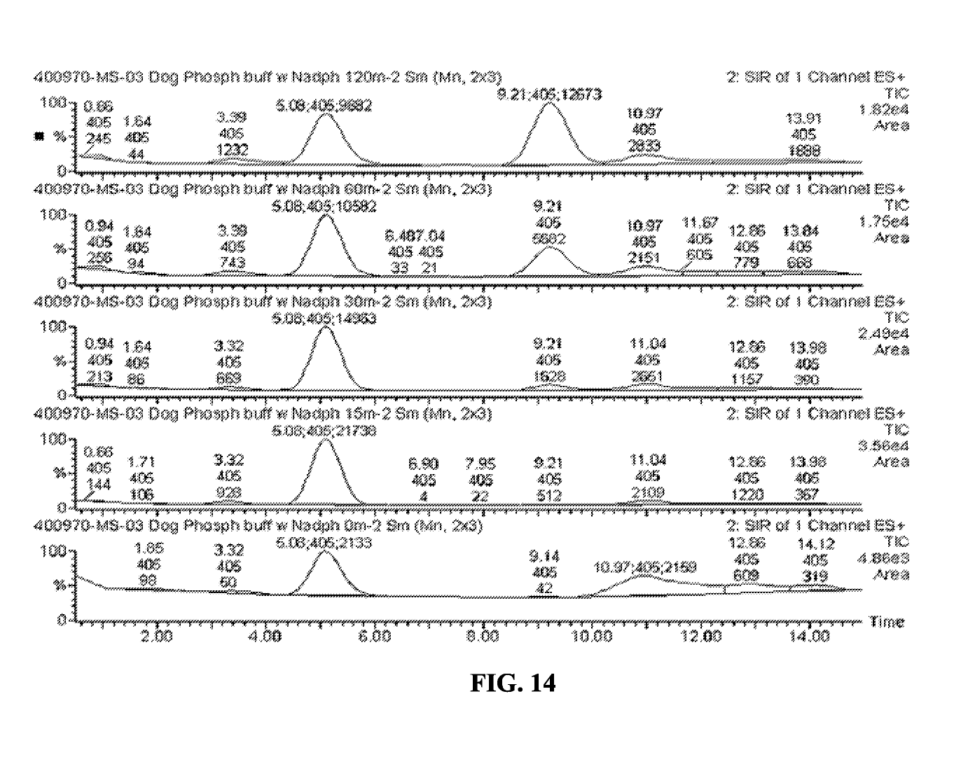

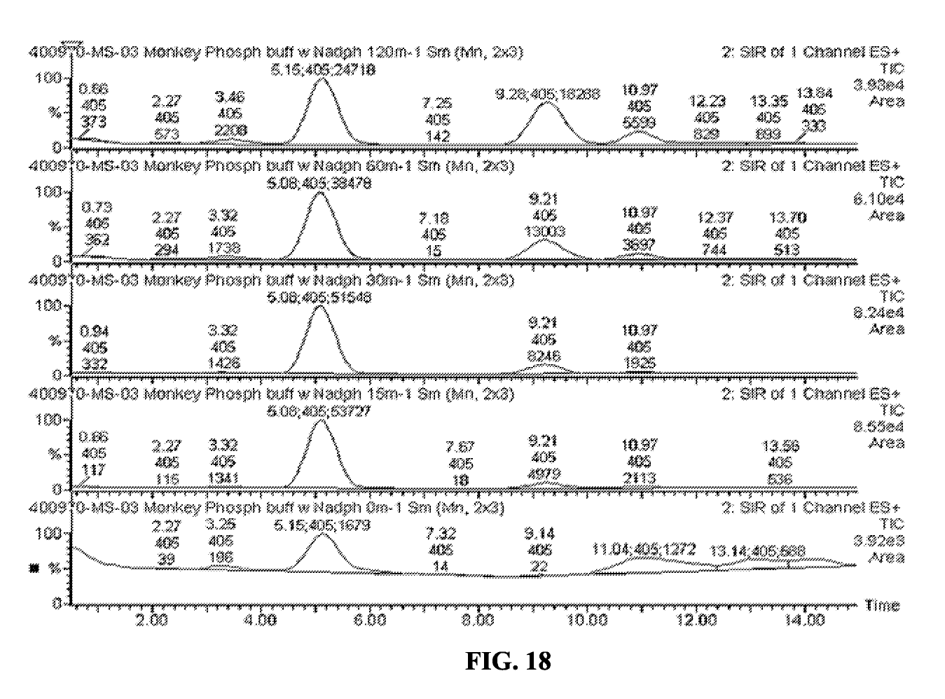

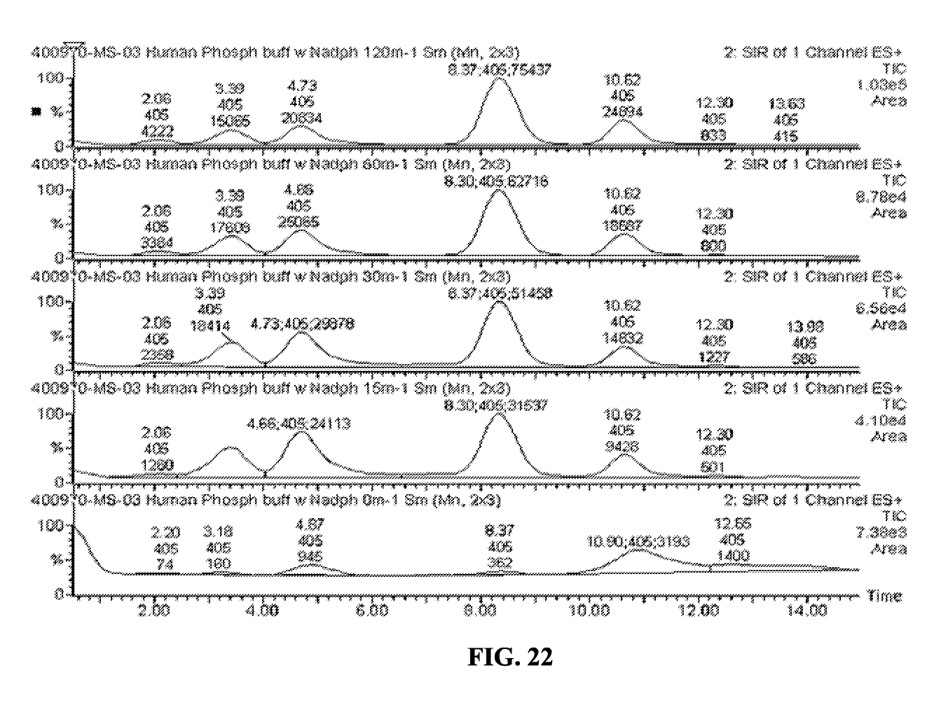

The concentrations of Compound (1) and its metabolites following incubation with pooled rat, dog, monkey and human liver microsomes in the presence or in the absence of the NADPH-generating system for various times are presented in FIG. 10, FIG. 14 FIG. 18, and FIG. 22 respectively.

The results obtained from the incubation of a target concentration of 10 μM Compound (1) with pooled rat liver microsomes in 0.1 M phosphate buffer demonstrated that, Compound (1) was relatively stable over 120 minutes of incubation in the presence of the NADPH-generating system with ˜86% of recovery compared to the 0 minute incubation (FIG. 10). Although no apparent depletion of Compound (1) was observed, five novels peaks (m/z 405 eluting at 3.3, 4.9 and 8.8 minutes, and m/z 421 eluting at 1.0 and 3.7 minutes) were detected. Potential metabolites with m/z 405 and 421 (e.g. mono and dihydroxylation) were observed after incubation with a target concentration of 10 μM Compound (1) in rat liver microsomes in an NADPH and time-dependent manner (FIG. 10 and FIG. 11).

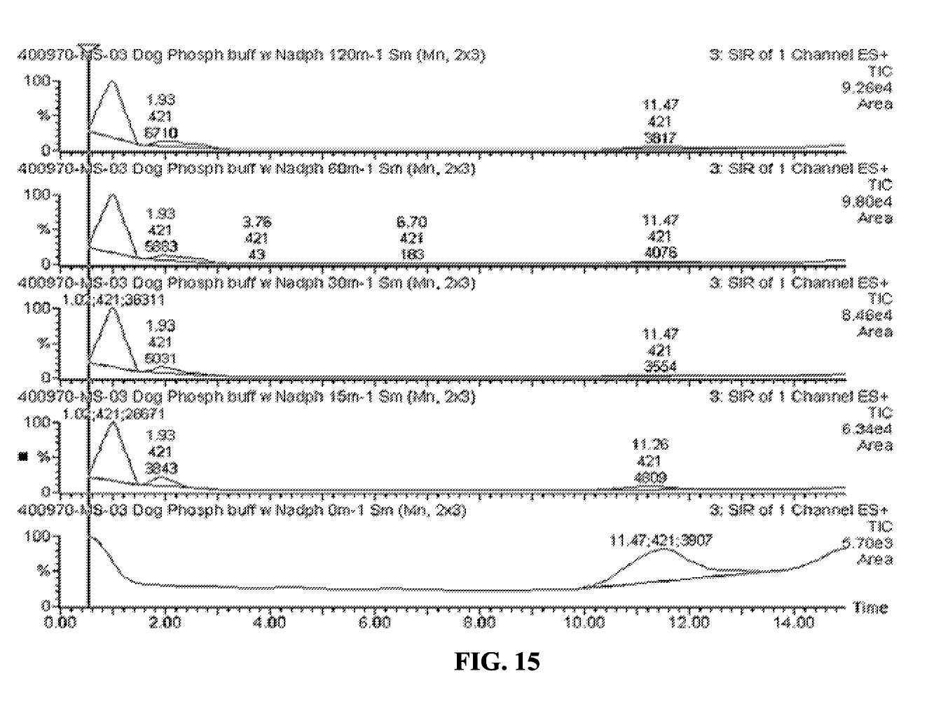

The incubation of a target concentration of 10 μM Compound (1) in dog liver microsomes in the presence of the NADPH-generating system and 0.1 M phosphate buffer demonstrated that the test article was metabolically stable for up 60 minutes. Following 120 minutes of incubation, depletion of ˜25% of Compound (1) was observed compared to the 0 minute incubation (FIG. 14). Novel peaks not present in the negative control (0 minute and/or without NADPH) samples were investigated and, similarly to rat liver microsomes, potential metabolites with m/z 405 and 421 (e.g. mono and dihydroxylation) were detected. Indeed, five novels peaks (m/z 405 eluting at ˜3.4, 5.1, and 9.2 minutes, and m/z 421 eluting at ˜1.0 and 1.9 minutes) were observed after incubation with a target concentration of 10 μM Compound (1) in dog liver microsomes in a time-dependent manner (FIG. 14 and FIG. 15).

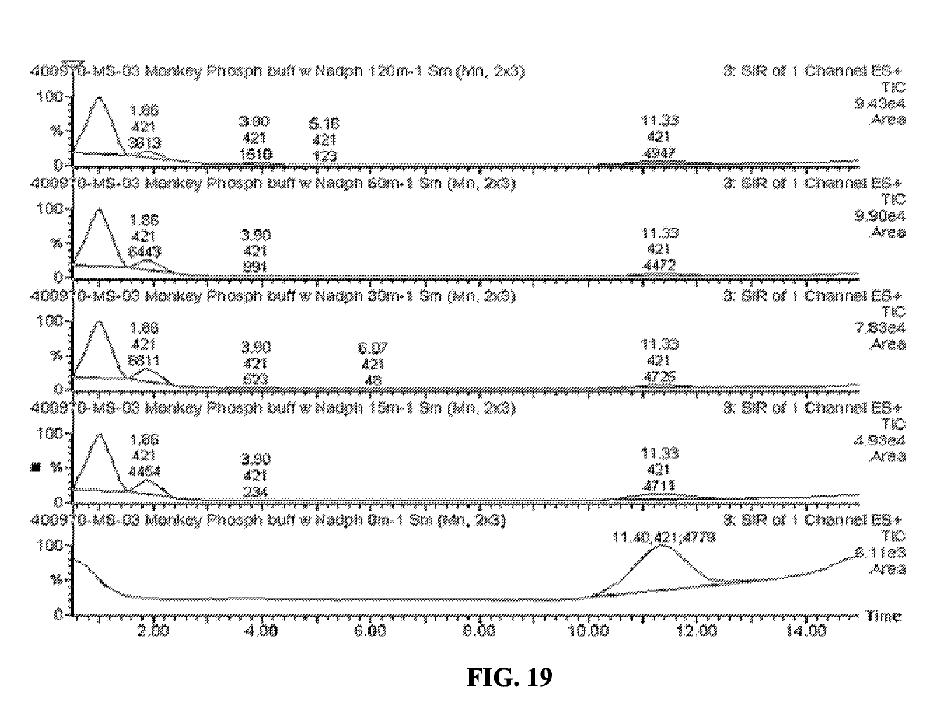

The results obtained from the incubation of a target concentration of 10 μM Compound (1) in phosphate buffer with monkey liver microsomes in the presence and in the absence of the NADPH-generating system demonstrated the test article was metabolized significantly. A time dependent depletion of Compound (1) of ˜15% for up to ˜55% over 120 minutes of incubation was observed (FIG. 18). The depletion of the parent compound correlated with the formation of novel peaks. Indeed, four novel peaks (m/z 405 eluting at ˜5.1, and ˜9.2 minutes, and m/z 421 eluting at ˜1.0 and 1.9 minutes) were observed after incubation with a target concentration of 10 μM Compound (1) in monkey liver microsomes in an NADPH and time-dependent manner (FIG. 18 and FIG. 19).

In the presence of 0.1 M phosphate buffer only, the incubation of Compound (1) in human liver microsomes demonstrated that the test article was metabolized at least up to 50% in an NADPH and time-dependent manner. The depletion of Compound (1) was concurrent with the formation of novel peaks. Using extracted ion chromatograms, potential metabolites with m/z 405 and 421 (e.g. mono and dihydroxylation) were investigated. The metabolism of Compound (1) in human liver microsomes in the tested condition resulted in seven novel peaks (m/z 405 eluting at ˜2.1, ˜3.4, ˜4.7, ˜8.4 and 10.6 minutes and m/z 421 eluting at ˜1.0 and ˜3.6 minutes).

### Metabolic Stability of Compound (1) in 0.1 M Phosphate Buffer, 1 mM EDTA and 3 mM MgCl₂

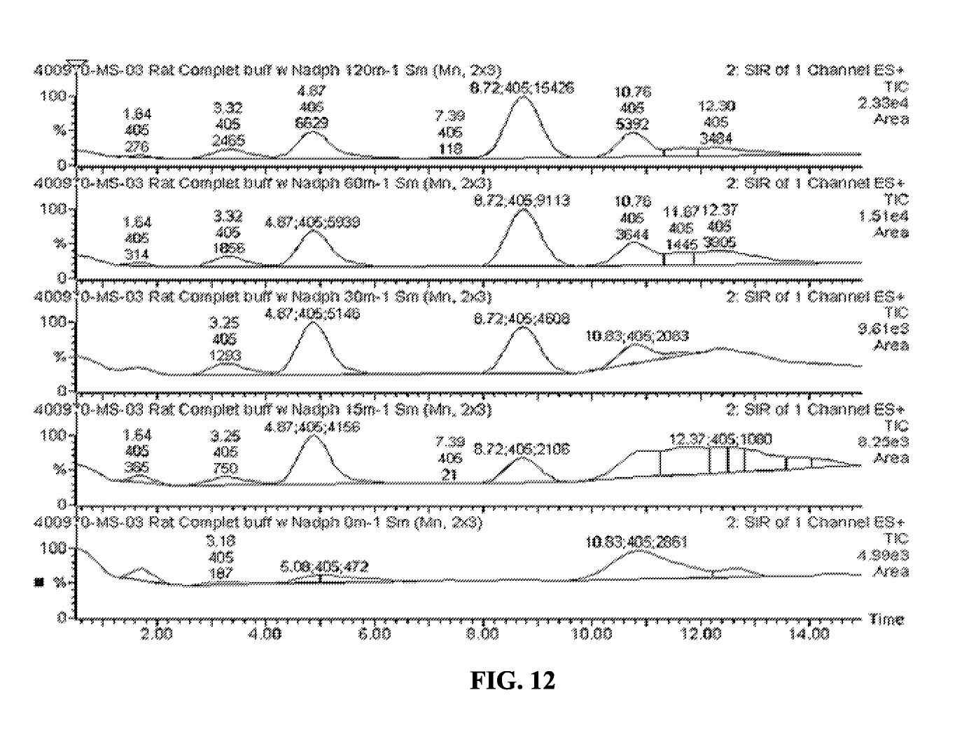

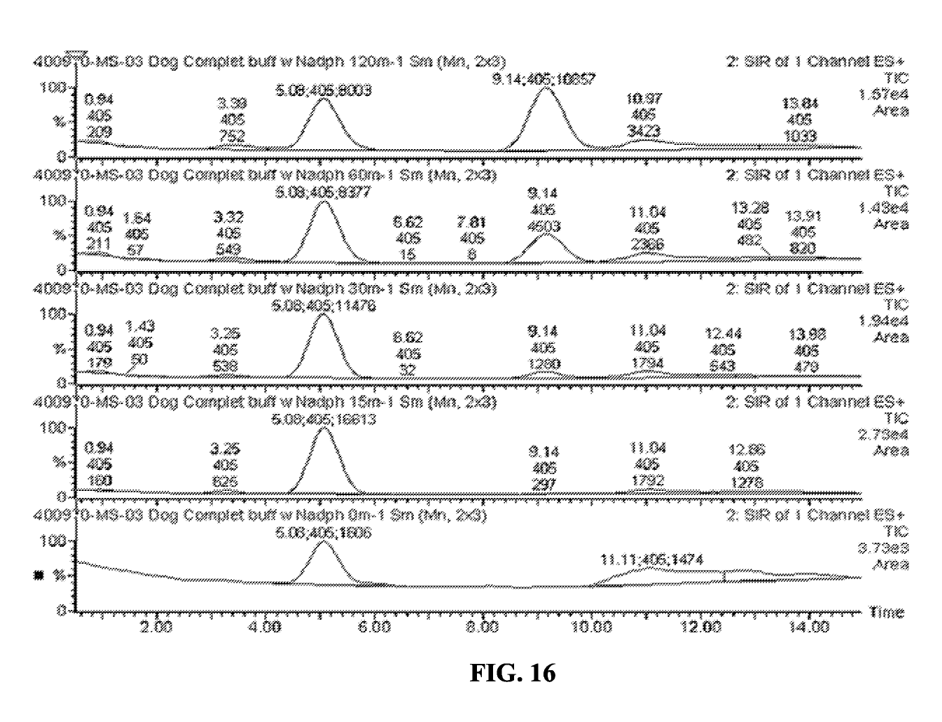

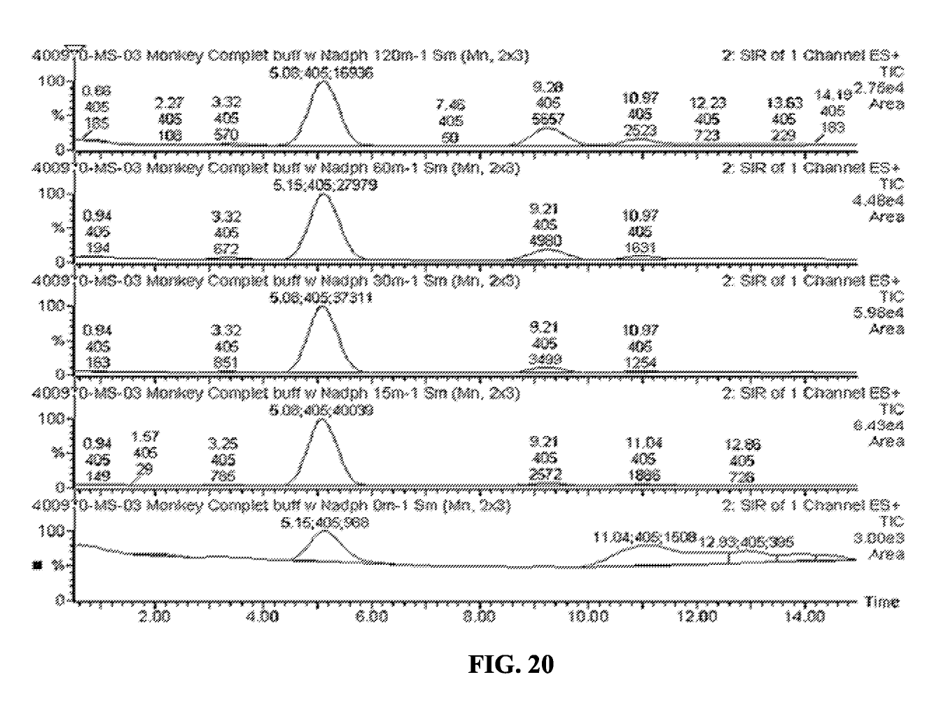

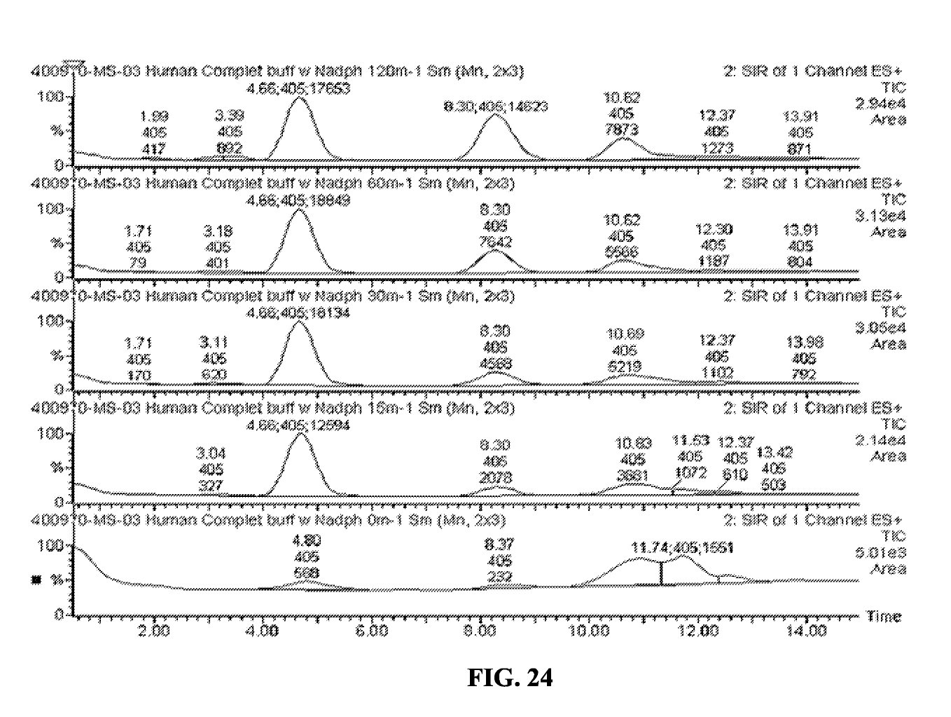

The concentrations of Compound (1) and its metabolites following incubation with pooled rat, dog, monkey and human liver microsomes in 0.1 M phosphate Buffer, 1 mM EDTA, 3 mM MgCl₂ and in the presence of or in the absence of the NADPH-generating system for various times are presented in FIG. 12, FIG. 16, FIG. 20 and FIG. 24, respectively.

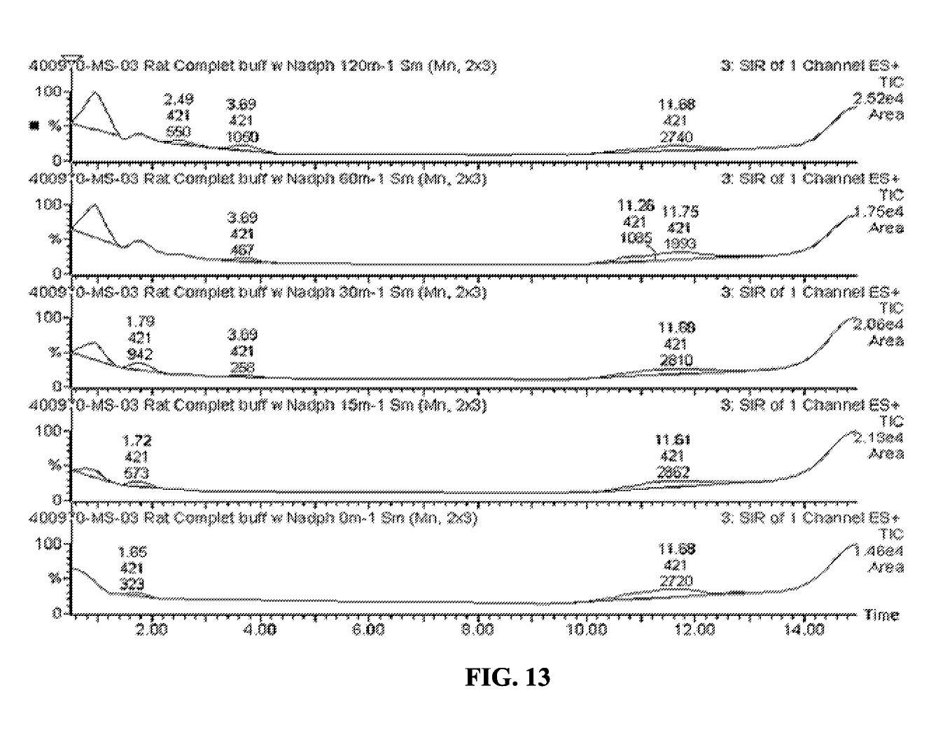

The results obtained from the incubation of a target concentration of 10 μM Compound (1) with pooled rat liver microsomes demonstrated that, Compound (1) was relatively stable over 60 minutes of incubation in the presence of the NADPH-generating system with ˜85% of recovery compared to the 0 minute incubation. However, at 120 minutes, the recovery of Compound (1) compared to the 0 minute slightly decreased to ˜79% (FIG. 12). Based on the total ion current chromatograms, any novel peaks not present in the negative control (0 minute and/or without NADPH) samples were investigated using extracted ion chromatograms of the potential metabolites from which m/z 405 and 421 (e.g. mono and dihydroxylation) were detected. Indeed, the small depletion of the parent compound correlated with the formation of five novels peaks (m/z 405 eluting at ˜3.3, ˜4.9, and ˜8.7 minutes and m/z 421 eluting at ˜1.0 and ˜3.7 minutes) that was time and NADPH-dependent (FIG. 12 and FIG. 13). Those peaks were also detected in the absence of MgCl₂ and EDTA incubations.

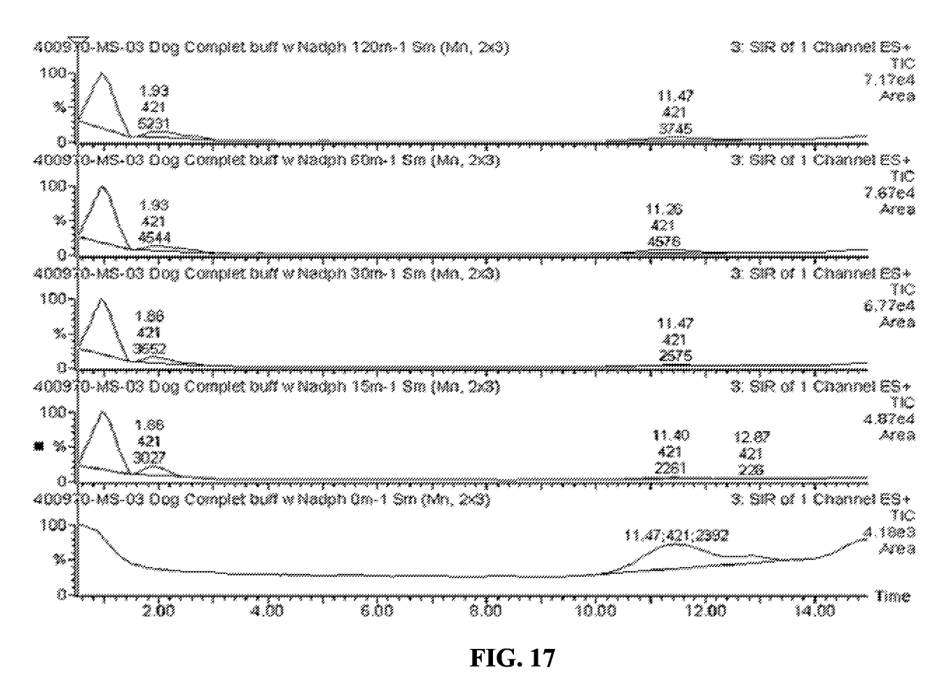

The incubation of a target concentration of 10 μM Compound (1) in dog liver microsomes in the presence of the NADPH-generating system demonstrated that the test article was metabolically stable in a manner similar to the rat. Compound (1) was stable for up to 60 minutes with a recovery of ˜85% compared to the 0 minute. Following 120 minutes of incubation, Compound (1) was metabolized at least up to 24% in an NADPH dependent manner (FIG. 14). Novel peaks not present in the negative control (0 minute and/or without NADPH) samples were investigated. Similarly to rat liver microsomes, potential metabolites with m/z 405 and 421 (e.g. mono and dihydroxylation) were detected. Indeed, four novels peaks (m/z 405 eluting at ˜5.1, and ˜9.1 minutes, and m/z 421 eluting at ˜1.0 and ˜1.9 minutes) were observed after incubation with a target concentration of 10 μM Compound (1) in dog liver microsomes in a time-dependent manner (FIG. 16, and FIG. 17). Those peaks were also detected in the absence of MgCl₂ and EDTA incubations.

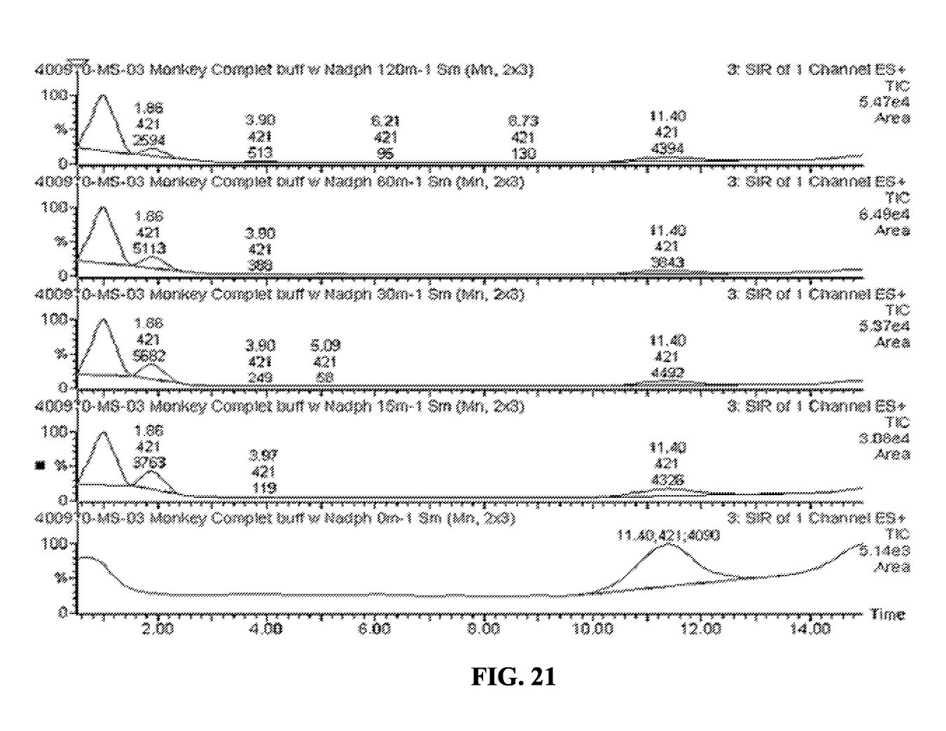

The results obtained from the incubation of a target concentration of 10 μM Compound (1) with monkey liver microsomes in the presence and in the absence of the NADPH-generating system demonstrated the test article was metabolically unstable over 120 minutes of incubation in a NADPH and time dependent manner. The depletion of the parent compound (decrease up to 37% in 120 minutes) correlate with the formation of novel peaks. Indeed, four novel peaks (405 eluting at ˜5.1 and ˜9.3 minutes and m/z 421 eluting at ˜1.0, and ˜1.9 minutes) were observed after incubation with a target concentration of 10 μM Compound (I) in monkey liver microsomes in an NADPH and time-dependent manner (FIG. 20 and FIG. 21). Those peaks were also detected in the absence of MgCl₂ and EDTA incubations.

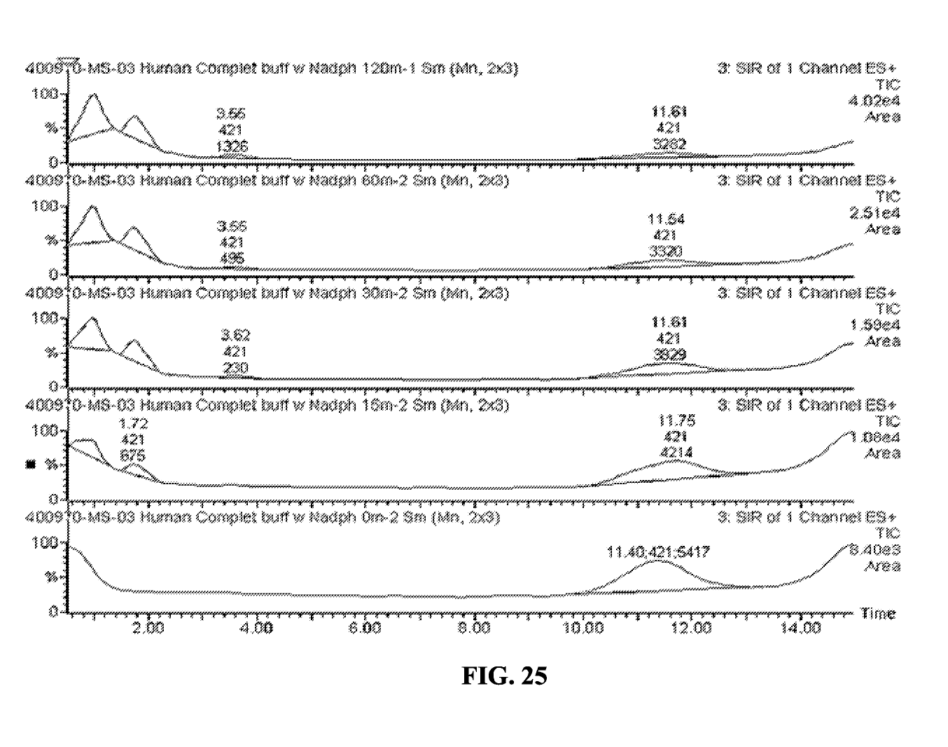

Following incubation in human liver microsome, Compound (1) was relatively stable over 120 minutes in the presence of the NADPH-generating system (recovery of ˜89%). However, the incubation of 10 μM of Compound (1) with human liver microsomes resulted in the formation of six novel peaks (m/z 405 eluting at ˜4.7, ˜8.3 and 10.6 minutes and m/z 421 eluting at ˜1.0, ˜1.7 and ˜3.6 minutes) in a time-dependent manner (FIG. 24 and FIG. 25). Those peaks were also detected in the absence of MgCl₂ and EDTA incubations.

Conclusions Drawn from Incubation Studies

The metabolic stability of a target concentration of 10 μM Compound (1) in pooled mixed gender rat, dog, monkey and human liver microsomes over 120 minutes of incubation was conducted in the presence and in the absence of EDTA and MgCl₂. The depletion of the parent compound and the formation of metabolites in a time-dependent manner were explored in the presence or absence of an NADPH-generating system.

The results obtained from the incubation of a target concentration of 10 μM of Compound (1) in the presence of EDTA and MgCl₂, demonstrated that Compound (1) was metabolically stable in rat and dog liver microsomes for up to 60 minutes. Following 120 minutes of incubation, depletion of ˜21% and ˜24% of parent compound was observed in rat and dog liver microsomes respectively compared with a slight increase (in rat microsomes) and a significant decrease (˜21% in dog microsomes) for the negative controls without NADPH-GS, illustrating some variability in the assay. Compound (1) was less stable in monkey microsomes as demonstrated by ˜16% to ˜37% depletion following 30 to 120 minutes of incubation. However, this system showed an increase in Compound (1) concentrations in the absence of NADPH-GS (˜−14% and ˜−12% for 30 and 120 minutes of incubation). In contrast to monkey liver microsomes, Compound (1) was metabolically stable in human liver microsomes as demonstrated by its slight depletion of up to ˜11% over 120 minutes of incubation in the presence of NADPH-generating system but in the absence of NADPH-GS, human liver microsomes depleted Compound (1) slightly (˜7%). While the negative controls show standard assay variability, interpretation of metabolite stability trends can still be made.

In all species, the depletion of the parent compound correlated with the formation of novel peaks, putative metabolites (monooxidation). It is worth noting that, the formation of those putative metabolites was time and NADPH-dependent and thus, suggesting the involvement of either cytochrome P450 and/or flavin-containing monooxygenase (FMO) enzymes in the biotransformation of Compound (1).

In conclusion, Compound (1) was metabolically stable in rat, dog and human liver microsomes and less stable in monkey liver microsomes following incubation for up to 120 minutes in the presence of EDTA and MgCl₂.

### Kits/Articles of Manufacture

For use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprising a compound with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes, carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

### ILLUSTRATIVE EXAMPLES

The following examples provide illustrative methods for making and testing the effectiveness and safety of compounds of Formula (1). These examples are provided for illustrative purposes only and not to limit the scope of the claims provided herein. All of the methods disclosed and claimed herein can be made and executed without undue experimentation in light of the present disclosure. It will be apparent to those of skill in the art that variations may be applied to the methods and in the steps or in the sequence of steps of the method described herein without departing from the concept, spirit and scope of the claims. All such similar substitutes and modifications apparent to those skilled in the art are deemed to be within the spirit, scope and concept of the appended claims.

### Example 1

### Synthesis of Compounds of Formula (1)

### Example 1a

### Synthesis of 3α-Acetoxy-17-(1H-benzimidazol-1-yl)-16-formylandrosta-5,16-diene

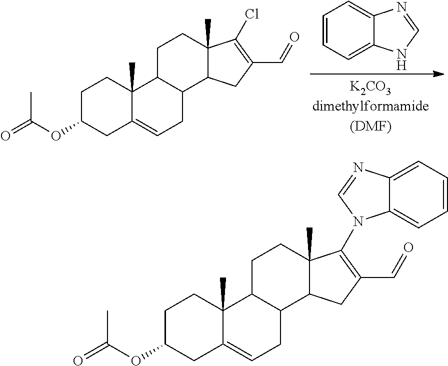

33.4 kg of 3α-acetoxy-17-chloro-16-formylandrosta-5,16-diene is mixed with benzimidazole and potassium carbonate in dimethylformamide (DMF) and is heated until the reaction is complete as determined by the amount of starting material remaining. After the reaction is complete, the reaction mixture is cooled and mixed with cooled water to quench the reaction. The solid is isolated from the quenched reaction mixture and washed sequentially with a mixture of DMF and water, water, dilute aqueous hydrochloric acid, water, dilute aqueous sodium hydrogen carbonate, and water. The intermediate product, 3α-Acetoxy-17-(1H-benzimidazol-1-yl)-16-formylandrosta-5,16-diene is subsequently dried.

### Example 1b

### Synthesis and Purification of 3α-Acetoxy-17-(1H-benzimidazol-1-yl)androsta-5,16-diene

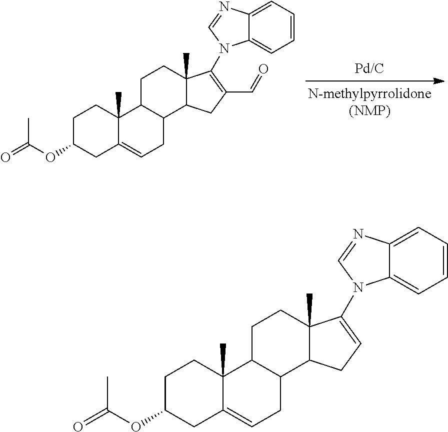

3α-Acetoxy-17-(1H-benzimidazol-1-yl)-16-formylandrosta-5,16-diene is mixed with about 10% palladium on carbon (Pd/C) in N-methylpyrrolidone (NMP) and is heated until the reaction is complete as determined by the 3α-Acetoxy-17-(1H-benzimidazol-1-yl)-16-formylandrosta-5,16-diene/3α-Acetoxy-17-(1H-benzimidazol-1-yl)androsta-5,16-diene ratio in the reaction mixture. After the reaction is complete, the reaction mixture is cooled. Magnesium sulfate is added, and the resulting mixture is filtered. Water is added to the filtrate and the resulting mixture is stirred. The solid, crude 3α-Acetoxy-17-(1H-benzimidazol-1-yl)androsta-5,16-diene is isolated from the water/NMP mixture, washed with a mixture of water and methanol, dried, and packaged.

The crude 3α-Acetoxy-17-(1H-benzimidazol-1-yl)androsta-5,16-diene is dissolved in ethyl acetate and clarified. The volume of this mixture is reduced by vacuum distillation. The resulting mixture is cooled, and the solid is isolated, washed with cold ethyl acetate, and dried under vacuum. In some embodiments, a sample is subjected to an in-process test to determine impurity levels. If the impurity levels are not acceptable, a recrystallization process is repeated.

### Example 1c

### Synthesis and Purification of 3α-Hydroxy-17-(1H-benzimidazol-1-yl)androsta-5,16-diene

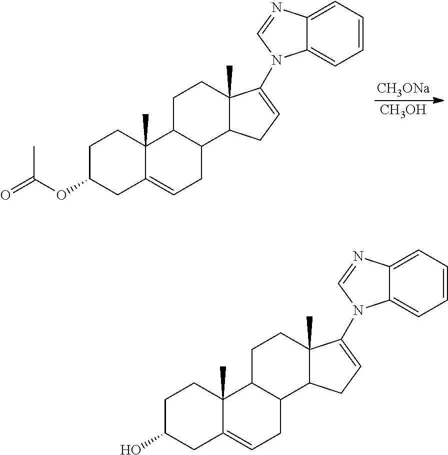

3α-Acetoxy-17-(1H-benzimidazol-1-yl)androsta-5,16-diene is mixed with sodium methoxide in methanol and is heated until the reaction is complete as determined by the amount of 3α-Acetoxy-17-(1H-benzimidazol-1-yl)androsta-5,16-diene remaining. After the reaction is complete, the reaction mixture is cooled and mixed with water to quench the reaction. The resulting slurry is stirred and cooled further. The solid, crude 3α-Hydroxy-17-(1H-benzimidazol-1-yl)androsta-5,16-diene is isolated from the quenched reaction mixture and washed with a mixture of methanol and water and then with water until the wash liquid is neutral, and subsequently dried and packaged.

The crude 3α-Hydroxy-17-(1H-benzimidazol-1-yl)androsta-5,16-diene is dissolved in a mixture of methanol and ethyl acetate and clarified. The product is transferred from the methanol/ethyl acetate solution to ethyl acetate alone by solvent exchange. The resulting mixture is cooled, and the solid is isolated, washed with cold ethyl acetate, and dried under vacuum. In some embodiments, a sample is subjected to an in-process test to determine impurity levels. If the impurity levels are not acceptable, a recrystallization process is repeated.

### Example 2

### Synthesis of (5S,10S,13S)-17-(1H-benzo[d]imidazol-1-yl)-10,13-dimethyl-4,5,6,7,8,9,10,11,12,13,14,15-dodecahydro-1H-cyclopenta[a]phenanthren-3(2H)-one (6)

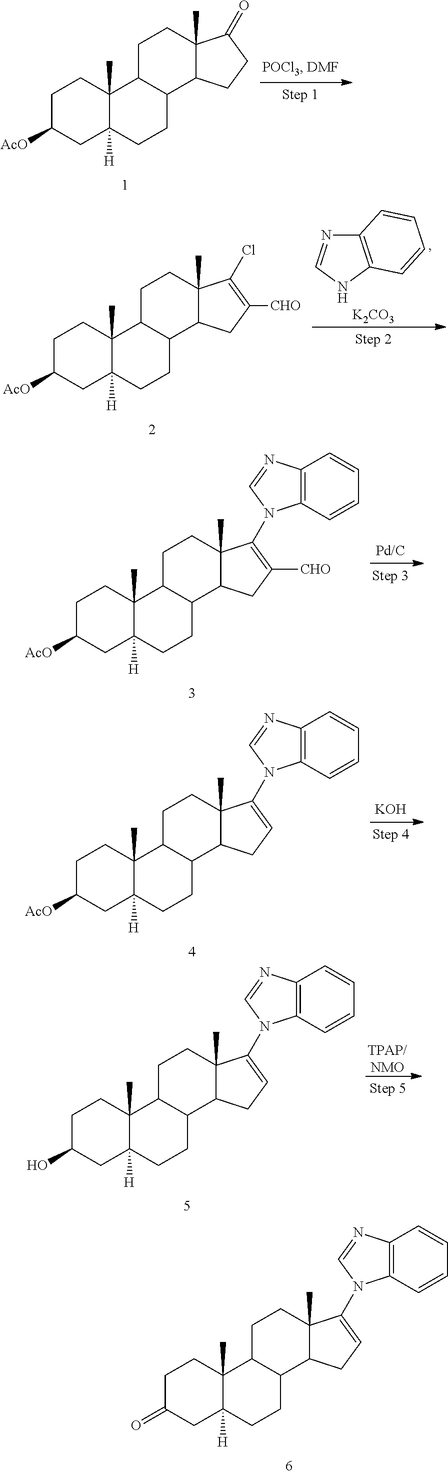

### Step 1: Preparation of (3S,5S,10S,13S)-17-chloro-16-formyl-10,13-dimethyl-2,3,4,5,6,7,8,9,10,11,12,13,14,15-tetradecahydro-1H-cyclopenta[a]phenanthren-3-yl acetate (2)

A solution of acetate 1 (3.0 g, 9.02 mmol) in anhydrous chloroform (60 mL) was added dropwise to a cold (0° C.) and stirred solution of phosphorous oxychloride (15.0 mL) and dimethylformamide (15.0 mL) under nitrogen. The mixture was allowed to warm to 25° C., then heated to reflux for 5 h, and then stirred at 50° C. overnight. The resultant mixture was concentrated under reduced pressure, poured onto ice, and extracted with ethyl acetate. The combined extracts were washed with water, brine, and dried (Na₂SO₄), and solvent was removed under reduced pressure to give a white solid. Purification by flash chromatography using 1-10% EtOAc/hexanes gave compound 2 (2.59 g, 81%). ¹H NMR (300 MHz, CDCl₃) δ 0.85 (s, 3H), 0.95 (s, 3H), 0.96-1.55 (m, 11H), 1.60-173 (m, 5H), 1.80-1.84 (m, 2H), 1.97-2.06 (m, 1H), 2.0 (s, 3H), 2.52 (m, 1H), 4.67 (m, 1H), 9.96 (s, 1H).

### Step 2: Preparation of (3S,5S,10S,13S)-17-(1H-benzo[d]imidazol-1-yl)-16-formyl-10,13-dimethyl-2,3,4,5,6,7,8,9,10,11,12,13,14,15-tetradecahydro-1H-cyclopenta[a]phenanthren-3-yl acetate (3)

A mixture of compound 2 (2.58 g, 6.80 mmol), benzimidazole (2.41 g, 20.4 mmol), and potassium carbonate (3.4 g, 24.6 mmol) in dry DMF (22 mL) was heated at 25° C. under N₂ for 1 h. The mixture was cooled to 25° C. and added to water, and the solid obtained was extracted with EtOAc. The combined extracts were washed with water, brine, and dried (Na₂SO₄), and solvent was remove under reduced pressure to give a brown solid. Purification by flash chromatography using 1-3% MeOH/CH₂Cl₂ gave compound 3 as a pale yellow solid (3.0 g, quant.) ¹H NMR (300 MHz, CDCl₃) δ 0.86 (s, 6H), 0.89-1.6 (m, 10H), 1.61-1.80 (m, 8H), 2.01 (s, 3H), 2.24-2.33 (m, 1H), 2.75 (dd, J=15.1, 6.06 Hz, 1H), 4.68 (m, 1H), 7.33 (m, 3H), 7.84 (m, 1H), 7.86 (s, 1H), 9.56 (s, 1H). APCI⁺=461.

### Step 3: Preparation of (3S,5S,10S,13S)-17-(1H-benzo[d]imidazol-1-yl)-10,13-dimethyl-2,3,4,5,6,7,8,9,10,11,12,13,14,15-tetradecahydro-1H-cyclopenta[a]phenanthren-3-yl acetate (4)

A solution of compound 3 (1.5 g, 3.0 mmol) in dry benzonitrile (8 mL) was refluxed in the presence of Pd/C (10 wt %, 750 mg) for 16 h. After cooling to 25° C., the catalyst was removed by filtration through a pad of Celite. The filtrate was evaporated, and the residue was purified by flash chromatography using 1% MeOH/CH₂Cl₂ to give compound 4 as a pale yellow solid (0.7 g, 50%). ¹H NMR (300 MHz, CDCl₃) δ 0.86 (s, 3H), 0.96 (s, 3H), 0.78-0.9 (m, 1H), 1.0-1.51 (m, 2H), 1.27-1.83 (m, 15H), 2.03 (s, 3H), 2.10-2.18 (m, 1H), 2.34-2.42 (m, 1H), 4.68 (m, 1H), 5.96 (s, 1H), 7.27 (m, 2H), 7.45 (m, 1H), 7.80 (m, 1H), 7.95 (s, 1H). APCI⁺=433.

### Step 4: Preparation of (3S,5S,10S,13S)-17-(1H-benzo[d]imidazol-1-yl)-10,13-dimethyl-2,3,4,5,6,7,8,9,10,11,12,13,14,15-tetradecahydro-1H-cyclopenta[a]phenanthren-3-ol (5)

To a solution of acetate 4 (450 mg, 1.61 mmol) in methanol (11.0 mL) at 0° C. was added a solution of KOH in methanol (10%, 4.3 mL) dropwise. The mixture was allowed to warm to 25° C. and was stirred overnight. The solvent was evaporated under reduced pressure and to the residue was added water, and the mixture was extracted with ethyl acetate. The organic phase was washed with water, brine, and dried (Na₂SO₄). The solvent was removed under reduced pressure to obtain crude material, which was purified by flash chromatography with 100% CH₂Cl₂ and 1-2% MeOH/CH₂Cl₂ to isolate to 5 as a pale yellow solid (400 mg, 63%). ¹H NMR (300 MHz, CDCl₃) δ 0.85 (s, 3H), 0.97 (s, 3H), 0.78-0.81 (m, 1H), 0.99-1.50 (m, 10H), 1.60-1.90 (m, 8H), 2.11-2.20 (m, 1H), 2.34-2.41 (m, 1H), 3.62 (m, 1H), 5.95 (dd, J=1.6, 3.3 Hz, 1H), 7.28 (m, 2H), 7.47 (m, 1H), 7.80 (m, 1H), 7.94 (s, 1H). HPLC=98%. APCI⁺=391.

### Step 5: Preparation of (5S,10S,13S)-17-(1H-benzo[d]imidazol-1-yl)-10,13-dimethyl-4,5,6,7,8,9,10,11,12,13,14,15-dodecahydro-1H-cyclopenta[a]phenanthren-3(2H)-one (6)

To a solution of 5 (150 mg, 0.38 mol) in a mixture of dichloromethane (4.0 mL) and acetonitrile (0.4 mL) was added N-methylmorpholine-N-oxide (NMO, 108 mg, 0.92 mmol), molecular sieves (4 Å, 300 mg) and tetrapropylammonium perruthenate (TPAP, 14 mg, 0.04 mmol). The mixture was stirred at 25° C. for 4 h. The reaction mixture was filtered through a pad of Celite and the filtrate was concentrated to a black residue, which was purified by flash chromatography using 100% CH₂Cl₂, then 1-2% MeOH/CH₂Cl₂ as eluent to isolate 6 as an off-white solid (77 mg, 52%). ¹H NMR (300 MHz, CDCl₃) δ 0.99 (s, 3H), 1.05 (s, 3H), 1.07-1.15 (m, 2H), 1.30-1.52 (m, 4H), 1.62-1.85 (m, 6H), 1.96-2.43 (m, 8H), 5.96 (dd, J=1.6, 3.3 Hz, 1H), 7.28 (m, 2H), 7.47 (m, 1H), 7.80 (m, 1H), 7.93 (s, 1H). HPLC=96%. APCI⁺=389.

### Example 3

### Synthesis of (5R,10S,13S)-17-(1H-benzo[d]imidazol-1-yl)-10,13-dimethyl-4,5,6,7,8,9,10,11,12,13,14,15-dodecahydro-1H-cyclopenta[a]phenanthren-3(2H)-one (7)

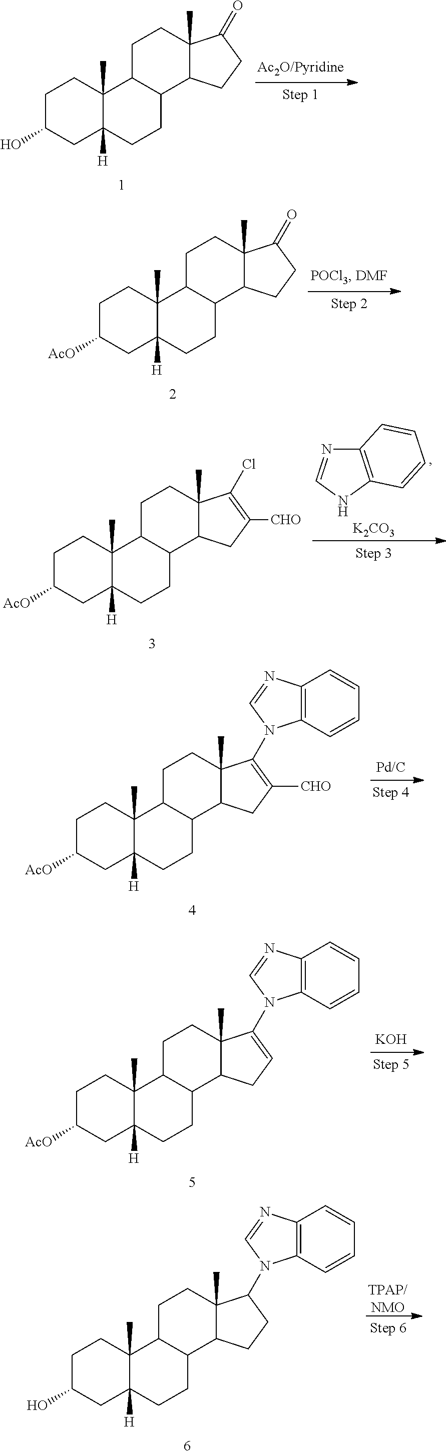

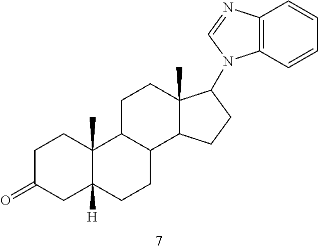

### Step 1: Preparation of (3R,5R,10S,13S)-10,13-dimethyl-17-oxohexadecahydro-1H-cyclopenta[a]phenanthren-3-yl acetate (2)

To a solution of alcohol 1 (3.0 g, 10.33 mmol) in pyridine (30 mL) at 0° C. under N₂ was added acetic anhydride (4.22 g, 41.32 mmol) dropwise, then the mixture was allowed to warm to 25° C. and was stirred at 25° C. overnight. Water was added to the reaction mixture and the resultant mixture was diluted with EtOAc. The organic phase was separated, and the aqueous phase was extracted with EtOAc. The combined organic phase was washed successively with 1 N HCl, saturated sodium bicarbonate, water, and brine. The organic phase was dried (Na₂SO₄) and evaporated to isolate the desired acetate 2 as a white solid (3.45 g, quant.). ¹H NMR (300 MHz, CDCl₃) δ 0.84 (s, 3H), 0.95 (s, 3H), 1.0-1.58 (m, 13H), 1.60-1.74 (m, 2H), 1.78-1.98 (m, 5H), 2.0 (s, 3H), 2.10 (m, 1H), 2.45 (m, 1H), 4.71 (m, 1H).

### Step 2: Preparation of (3R,5R,10S,13S)-17-chloro-16-formyl-10,13-dimethyl-2,3,4,5,6,7,8,9,10,11,12,13,14,15-tetradecahydro-1H-cyclopenta[a]phenanthren-3-yl acetate (3)

A solution of acetate 2 (1.5 g, 4.51 mmol) in anhydrous chloroform (30 mL) was added dropwise to a cold (0° C.) and stirred solution of phosphorous oxychloride (7.5 mL) and dimethylformamide (7.5 mL) under nitrogen. The mixture was allowed to warm to 25° C., was heated to reflux for 5 h, and then was stirred at 50° C. overnight. The resultant mixture was concentrated under reduced pressure and poured onto ice, then extracted with ethyl acetate. The combined extracts were washed with water, brine, and dried (Na₂SO₄), and the solvent was removed under reduced pressure to give a white solid. Purification by flash chromatography using 1-10% EtOAc/hexanes gave compound 3 (1.17 g, 68%). ¹H NMR (300 MHz, CDCl₃) δ 0.94 (s, 3H), 0.96 (s, 3H), 1.0-1.58 (m, 13H), 1.60-2.0 (m, 5H), 1.98-2.1 (m, 1H), 2.0 (s, 3H), 2.51 (m, 1H), 4.71 (m, 1H), 9.97 (s, 1H).

### Step 3: Preparation of (3R,5R,10S,13S)-17-(1H-benzo[d]imidazol-1-yl)-16-formyl-10,13-dimethyl-2,3,4,5,6,7,8,9,10,11,12,13,14,15-tetradecahydro-1H-cyclopenta[a]phenanthren-3-yl acetate (4)

A mixture of compound 3 (1.17 g, 3.08 mmol), benzimidazole (1.09 g, 9.22 mmol), and potassium carbonate (1.54 g, 11.1 mmol) in dry dimethylformamide (10 mL) was heated at 80° C. under N₂ for 1 h. The mixture was cooled to 25° C. and added to water, and the solid obtained was extracted with EtOAc. The combined extracts were washed with water, brine, and dried (Na₂SO₄), and the solvent was removed to give a brown solid. Purification by flash chromatography using 1-3% MeOH/CH₂Cl₂ gave compound 4 as a pale yellow solid (1.40 g, quant.). ¹H NMR (300 MHz, CDCl₃) δ 0.94 (s, 6H), 0.92-1.5 (m, 6H), 1.50-1.59 (m, 2H), 1.60-1.90 (m, 10H), 2.03 (s, 3H), 2.24-2.33 (m, 1H), 2.75 (dd, J=15.1, 6.06 Hz, 1H), 4.74 (m, 1H), 7.33 (m, 3H), 7.84 (m, 1H), 7.86 (s, 1H), 9.59 (s, 1H). APCI⁺=461.

### Step 4: Preparation of (3R,5R,10S,13S)-17-(1H-benzo[d]imidazol-1-yl)-10,13-dimethyl-2,3,4,5,6,7,8,9,10,11,12,13,14,15-tetradecahydro-1H-cyclopenta[a]phenanthren-3-yl acetate (5)

A solution of compound 4 (700 mg, 1.51 mmol) in dry benzonitrile (3.4 mL) was refluxed in the presence of Pd/C (10 wt %, 350 mg) for 8 h. After cooling to RT, the catalyst was removed by filtration through a pad of Celite. The filtrate was evaporated, and the residue was purified by flash chromatography using 1% MeOH/CH₂Cl₂ to give compound 5 as a pale yellow solid (0.46 g, 71%). ¹H NMR (300 MHz, CDCl₃) δ 0.94 (s, 3H), 0.97 (s, 3H), 0.92-1.5 (m, 6H), 1.50-1.59 (m, 2H), 1.60-1.90 (m, 10H), 2.03 (s, 3H), 2.24-2.33 (m, 1H), 2.75 (dd, J=15.1, 6.06 Hz, 1H), 4.74 (m, 1H), 5.96 (s, 1H), 7.27 (m, 2H), 7.33 (m, 1H), 7.81 (m, 1H), 7.93 (s, 1H). APCI⁺=433.

### Step 5: Preparation of (3R,5R,10S,13S)-17-(1H-benzo[d]imidazol-1-yl)-10,13-dimethyl-2,3,4,5,6,7,8,9,10,11,12,13,14,15-tetradecahydro-1H-cyclopenta[a]phenanthren-3-ol (6)

To a solution of acetate 5 (450 mg, 1.04 mmol) in methanol (7.0 mL) at 0° C. was added a solution of KOH in methanol (10%, 2.8 mL) dropwise, and the mixture was allowed to warm to 25° C. and was stirred for 2 h. The solvent was evaporated to a residue under reduced pressure, and to the residue was added water. The resultant mixture was extracted with ethyl acetate. The organic phase was washed with water, brine, and dried (Na₂SO₄). The solvent was removed under reduced pressure to obtain crude material, which was purified by flash chromatography with 100% CH₂Cl₂, then 1-2% MeOH/CH₂Cl₂ to isolate to 6 as a pale yellow solid (250 mg, 63%). ¹H NMR (300 MHz, CDCl₃) δ 0.94 (s, 3H), 0.97 (s, 3H), 1.00-1.35 (m, 1H), 1.22-1.56 (m, 8H), 1.60-2.0 (m, 10H), 2.10-2.20 (m, 1H), 2.34-2.45 (m, 1H), 3.65 (m, 1H), 5.96 (dd, J=1.6, 3.3 Hz, 1H), 7.28 (m, 2H), 7.47 (m, 1H), 7.79 (m, 1H), 7.93 (s, 1H). HPLC=100%. APCI⁺=391.

### Step 6: Preparation of (5R,10S,13S)-17-(1H-benzo[d]imidazol-1-yl)-10,13-dimethyl-4,5,6,7,8,9,10,11,12,13,14,15-dodecahydro-1H-cyclopenta[a]phenanthren-3(2H)-one (7)

To a solution of 6 (130 mg, 0.33 mmol) in a mixture of dichloromethane (3.0 mL) and acetonitrile (0.33 mL) was added N-methylmorpholine-N-oxide (NMO, 94 mg, 0.8 mmol), molecular sieves (4 Å, 260 mg) and tetrapropylammonium perruthenate (TPAP, 12 mg, 0.034 mmol). The mixture was stirred at 25° C. for 4 h. The reaction mixture was filtered through a pad of Celite and the filtrate was concentrated to a black residue, which was purified by flash chromatography using 100% CH₂Cl₂, then 1-2% MeOH/CH₂Cl₂ as eluent to isolate 7 as an off-white solid (91 mg, 70%). ¹H NMR (300 MHz, CDCl₃) δ 0.99 (s, 3H), 1.07 (s, 3H), 1.20-1.54 (m, 4H), 1.62-1.78 (m, 4H), 1.80-1.92 (m, 4H), 1.96-2.20 (m, 3H), 2.15-2.25 (m, 2H), 2.28-2.5 (m, 2H), 2.73 (m, 1H), 5.96 (dd, J=1.6, 3.3 Hz, 1H), 7.28 (m, 2H), 7.47 (m, 1H), 7.79 (m, 1H), 7.93 (s, 1H). HPLC=99%. APCI⁺=389.

### Example 4

### (3S,10R,13S)-17-(1H-benzo[d]imidazol-1-yl)-10,13-dimethyl-2,3,6,7,8,9,10,11,12,13,14,15 dodecahydro-1H-cyclopenta[a]phenanthren-3-ol (3)

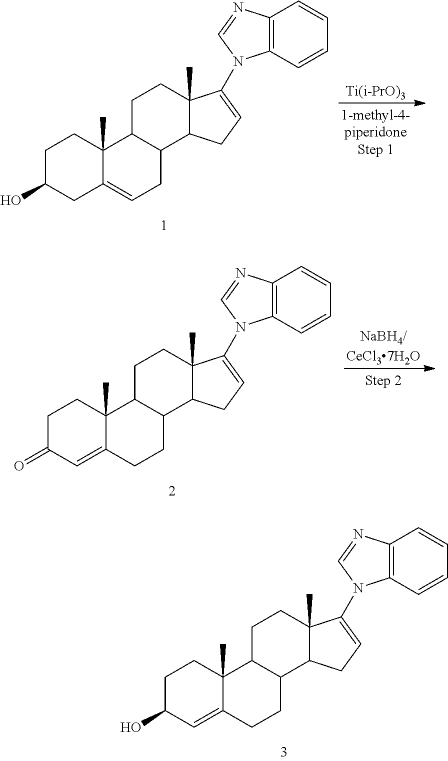

### Step 1: Preparation of (10R,13S)-17-(1H-benzo[d]imidazol-1-yl)-10,13-dimethyl-6,7,8,9,10,11,12,13,14,15-decahydro-1H-cyclopenta[a]phenanthren-3(2H)-one (2)

Ketone 2 was prepared by following the procedure described in WO 2006/093993.

### Step 2: Preparation of (3S,10R,13S)-17-(1H-benzo[d]imidazol-1-yl)-10,13-dimethyl-2,3,6,7,8,9,10,11,12,13,14,15 dodecahydro-1H-cyclopenta[a]phenanthren-3-ol (3)

To a solution of ketone 2 (150 mg, 0.39 mmol) in methanol (4 mL) under N₂ was added cerium chloride heptahydrate (145 mg, 0.39 mmol), and the solution was cooled to −20° C. Sodium borohydride (7.4 mg, 0.195 mmol) was then added, and the mixture was stirred at −20° C. for 0.5 h and then at −15° C. for 0.5 h. Water was added to the reaction mixture, followed by EtOAc. The organic phase was separated, and the aqueous phase was extracted with EtOAc. The combined organic phase was washed with brine, dried (Na₂SO₄) and evaporated to isolate the desired product 3 as a white solid (150 mg, quant.). ¹H NMR (300 MHz, CDCl₃) δ 0.90-1.06 (m, 2H), 0.99 (s, 3H), 1.08 (s, 3H), 1.27-1.50 (m, 3H), 1.57-1.87 (m, 7H), 1.90-1.98 (m, 1H), 2.05-2.42 (m, 4H), 4.14 (m, 1H), 5.32 (d, J=1.4 Hz, 1H), 5.96 (dd, J=3.03, 1.4 Hz, 1H), 7.29 (m, 2H), 7.47 (m, 1H), 7.80 (m, 1H), 7.96 (s, 1H). HPLC=97%, APCI⁺=389.

### Example 5

### Pharmaceutical Compositions

### Example 2a

### Oral Composition

To prepare a pharmaceutical composition for oral delivery, a compound of Formula (1) is micronized to have a bulk density of about 0.20 g/mL and a tap density of about 0.31 g/mL. 90 mg of micronized compound is pack-filled into size “3” capsules suitable for oral administration.

### Example 2b

### Oral Composition

To prepare a pharmaceutical composition for oral delivery, a compound of Formula (1) is micronized to have a bulk density of about 0.20 g/mL and a tap density of about 0.31 ng/mL. 325 mg of micronized compound is pack-filled into size “00” capsules suitable for oral administration.

### Example 2c

### Oral Composition

To prepare a pharmaceutical composition for oral delivery, 90 mg of a compound of Formula (1) is mixed with 200 mg of lactose and 1% magnesium stearate. The mixture is blended and directly compressed into a tablet suitable for oral administration.

### Example 6

### In Vitro Pharmacological Studies

### Example 3a

### Androgen Receptor Binding Assay

Androgen receptor (AR) competition binding is determined using radiolabeled R1881 (an androgen agonist) in androgen sensitive human prostate cancer cell line (LNCaP) cells that express the mutated AR (IC₅₀ of 384 nM), and in cells that express the wild-type AR (IC₅₀ of 845 nM). A compound of Formula (1) is added to the cells in increasing concentrations. The amount of radiolabeled R1881 is measured as a measuring of competition binding to the AR.

### Example 3b

### Inhibition of Lyase Activity

Intact CYP17 expressed by transfected E. coli is isolated and purified as an enzyme source. Radiolabelled 17-α-hydroxypregnenolone as the substrate. CYP17 activity is measured by the amount of tritiated acetic acid formed during the cleavage of the C-21 side chain of the substrate. A compound of Formula (1) is added to the reaction in increasing concentrations to assess inhibitory effects on CYP17 cleavage of substrate.

### Example 3c

### Inhibition of Testosterone-Induced Proliferation of Prostate Cancer Cell Lines

Human prostate cancer cell lines (LNCaP and LAPC-4) are grown in culture and stimulated with 1 nM dihydrotestosterone (DHT). This concentration of DHT stimulates the proliferation of prostate cancer cells. A compound of Formula (1) is added to the cells in increasing concentrations to assess the effect on proliferation.

### Example 3d

### Degradation of Androgen Receptor (AR) Protein in Prostate Cancer Cell Lines

Cycloheximide is added to human prostate cancer cell (LNCaP) to inhibit all protein synthesis in the cultured cells. Cycloheximide treatment alone reduced AR levels in a time-dependent fashion when protein extracts were probed with monoclonal antibodies directed against the AR protein. A compound of Formula (1) is added to the cells in increasing concentrations to determine whether the addition results in a significantly greater rate of decrease of AR protein with time in culture.

### Example 7

### In Vivo Pharmacological Studies

### Example 4a

### Inhibition of Growth of Human Prostate Cancer Xenografts in Severely Compromised Immunodeficient (SCID) Mice

Xenografts of LAPC4 prostate cancer cell tumors are implanted in SCID mice. Tumor-bearing mice receive twice daily subcutaneous (SC) administration of 50 mg/kg body weight (BW) a compound of Formula (1). Tumor size is measured weekly and compared with control mice that receive vehicle, Casodex® or castration only.

### Example 8

### Detection of Metabolites of β-Hydroxy-17(1H-benzimidazol-1-yl)androsta-5,16-diene

Test System

3β-Hydroxy-17(1H-benzimidazol-1-yl)androsta-5,16-diene is incubated with pooled rat, dog, monkey and human liver microsomes at a protein concentration of 0.6 mg/mL. Incubation mixture contains either 0.1 M potassium phosphate buffer pH 7.4 or the incubation buffer. Following two minutes of pre-incubation of the incubation mixture at ˜37° C., the reaction is started by the addition of an NADPH-generating system (NADPH-GS) (1 mM NADP+, 5 mM glucose-6-phosphate, 1.0 unit/mL glucose-6-phosphate dehydrogenase) or 0.1 M phosphate buffer. At the appropriate time points (0, 15, 30, 60 and 120 minutes), reactions are stopped by the addition of appropriate stop solutions (0.1% formic acid in acetonitrile). The samples are centrifuged at approximately 10 000×g for 10 minutes at 20° C. An amount of supernatant from each sample is transferred to pre-labeled HPLC vials for analysis. Samples are analyzed by LC/MS to monitor the remaining parent compound or formation of potential metabolites. On each occasion, blank sample containing TOK-001 but without microsomes, is included.

Results

LC/MS of 3β-Hydroxy-17(1H-benzimidazol-1-yl)androsta-5,16-diene shows a m/z of 389. In all species, the depletion of 3β-Hydroxy-17(1H-benzimidazol-1-yl)androsta-5,16-diene by the liver microsomes correlates with the formation of novel peaks at m/z 405 and 421 compared with the negative controls, indicating putative metabolites (e.g., mono and dihydroxylation).

### Example 9

### Verification of Metabolite Chemical Structures by HPLC-MS/MS

A HPLC-MS/MS method for the quantitation of the parent compound:

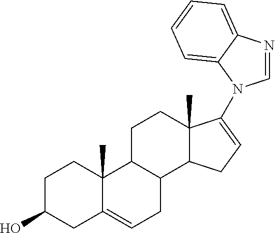

in plasma was installed and optimized to provide baseline resolution of possible metabolite peaks in non-human primate plasma samples. Several combinations of mobile phases and stationary phases, along with gradient modification, were evaluated. Improved resolution was obtained using an ACE 5 C18 stationary phase with a modified gradient.

The optimized method was qualified to provide assurance that the modifications that were made to the method did not adversely impact its performance for quantitation of the parent. The optimized method was determined to be linear over the range of 0.5-500 ng/mL parent. A total of 18 out of 18 standards over the linear range met the acceptance criteria of back-calculating to within ±15% of their nominal concentrations and had CV's under 15% for each duplicate extraction.

Retained in vivo plasma samples from studies in dogs and monkeys were extracted according to the validated method and analyzed in full scan mode. Several species were identified with mass changes with respect to the parent, which corresponded to previously observed in vivo modifications.

A well-defined fragmentation pattern was obtained for the parent compound by direct infusion into the mass spectrometer. Samples of progressively lower concentration, prepared in both analytical diluent and human plasma, were analyzed to determine at what concentration the observed fragmentation pattern could be distinguished from background noise.

Well-defined patterns were obtained for samples containing 0.417 ng/mL of the parent in diluent, and 5-10 ng/mL of the parent in plasma.

The analysis of nine authentic standards of potential metabolites of the parent determined that two of the authentic standards, Std. 5 and Std. 7, did co-elute with one of the two metabolite peaks observed in samples from in vivo studies. None of the nine authentic standards matched the other observed metabolite peak.

Additional peaks were observed to elute or nearly co-elute with the parent compound during in vivo studies. These additional peaks were obtained through multiple reaction monitoring (MRM) using the same mass transition (389 m/z to 195 m/z), indicating that the peaks other than those of the analyte are likely to be isomers of the parent, produced by one or more double bond migrations and/or epimerization or migration of the secondary hydroxyl group. Potential isomeric metabolites of the parent were conceived and synthesized for use as comparator standards in analytical experiments. Such experiments required adequate resolution between the parent and the additional peaks for accurate and reproducible quantitation.

### HPLC-MS/MS Bioanalytical Method Optimization

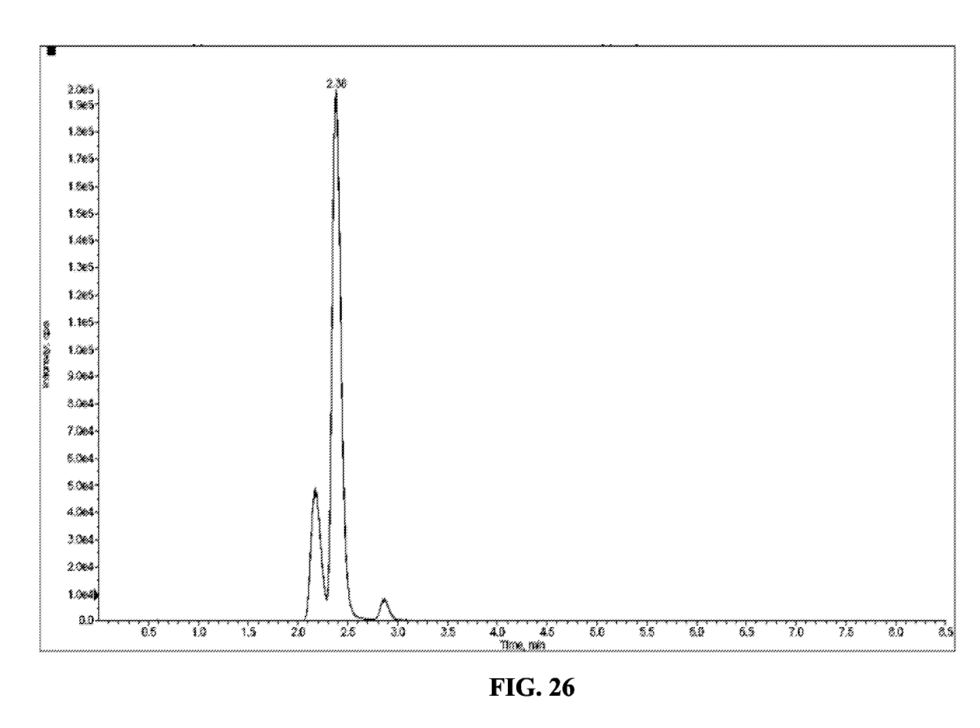

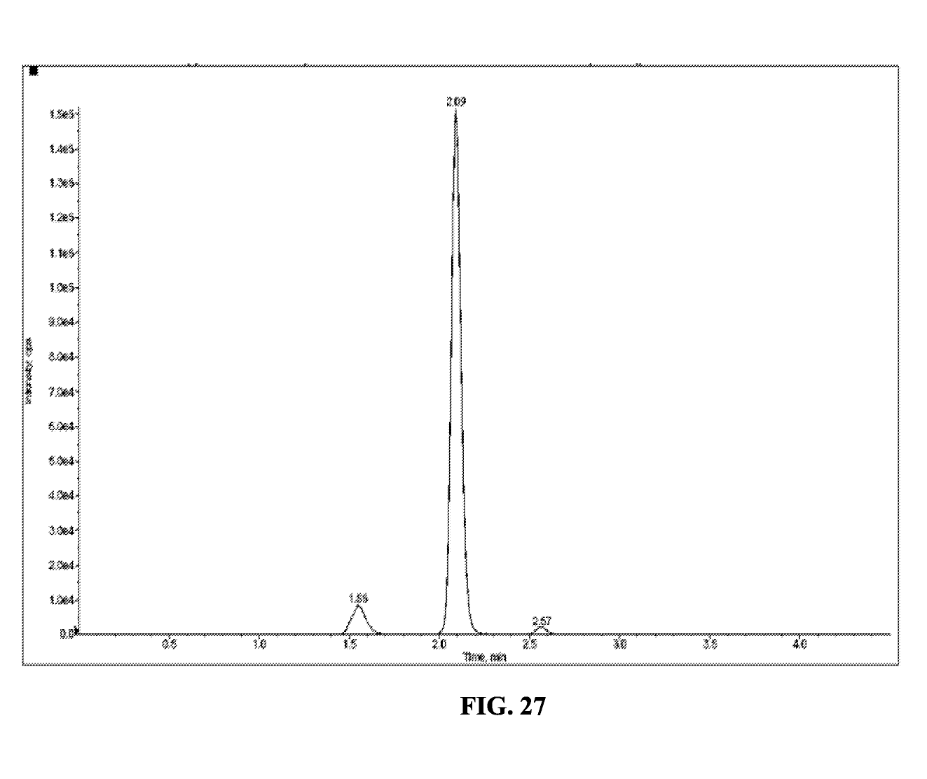

The initial HPLC-MS/MS method for the quantitation of the parent was installed on the HPLC-MS/MS platform. Dog and monkey plasma samples from previous studies were extracted according to the validated method and analyzed to reproduce the original chromatography. Sample chromatograms are illustrated in FIGS. 26 and 27.

A variety of analytical columns were screened, analyzing the extracted dog and monkey plasma samples, and were tested with the mobile phases: 0.1% formic acid in water and 0.1% formic acid in acetonitrile. The parameters of peak shape, peak area, and peak symmetry were assessed in the selection of the column. The table below presents the columns tested and a brief comment of the observed chromatography.

### Analytical Column Selection

| | | | Particle | | | |
|---|---|---|---|---|---|---|
| | | Pore | Size | Length | | |
| Manufacturer | Packing | Size (Å) | (μm) | (mm) | Part Number | Outcome |
| Ace (Mac-Mod) | C18 | 100 | 5 | 2.1 × 50 | ACE-121-0502 | improved resolution, |
| | | | | | | good peak shape |
| Ace (Mac-Mod) | C18 | 100 | 3 | 2.1 × 50 | ACE-111-0502 | no improvement |
| Ace (Mac-Mod) | C4 | 300 | 3 | 2.1 × 51 | ACE-213-0502 | poor resolution |
| Phenomenex | Synergi Polar RP | 80 | 4 | 2 × 30 | 00A-4336-B0 | no improvement |
| Phenomenex | Luna Phenyl-Hexyl | 100 | 5 | 2 × 30 | 00A-4257-B0 | reduced signal |
| Waters | Symmetry C18 | 100 | 3.5 | 2.1 × 50 | WAT200650 | poor resolution, |
| | | | | | | split peaks |

The most promising results were obtained using the ACE 5 C18 (p/n ACE-121-0502) column. Additional mobile phases were evaluated for their effectiveness in improving the resolution between observed peaks. The additional mobile phases included:
0.1% formic acid in water and 0.1% formic acid in methanol (MeOH); and
2 mM ammonium acetate, 0.1% formic acid, 95:5 H₂O:MeOH and 2 mM ammonium acetate, 0.1% formic acid, 5:95 H₂O:MeOH.

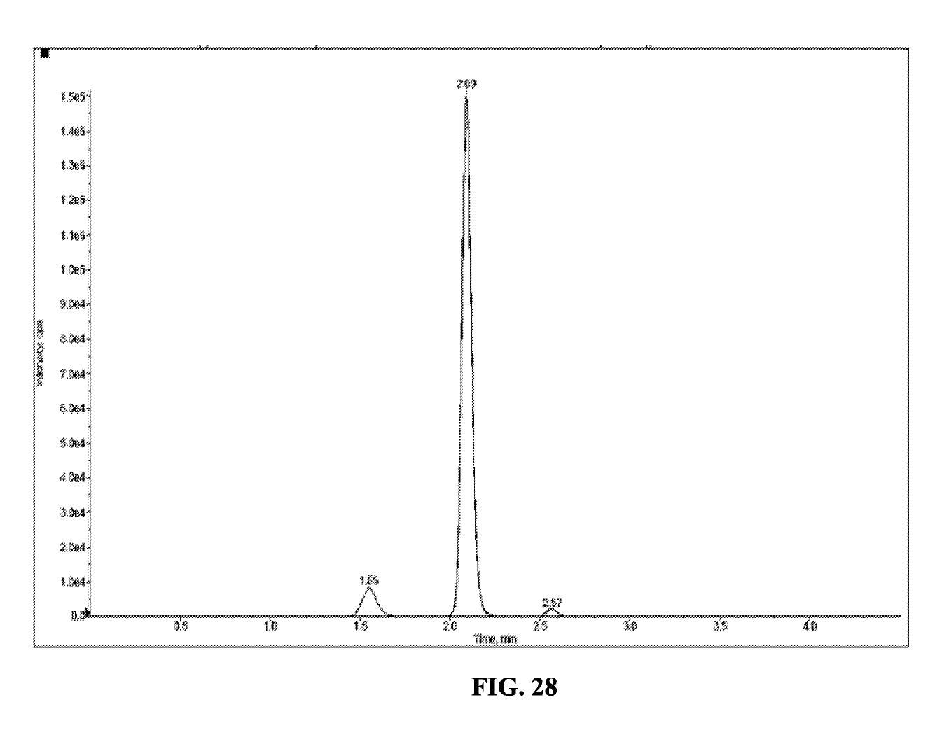

Similar results were obtained with all three sets of mobile phases. To minimize the number of parameters that were changed from the validated method, the original mobile phases (0.1% formic acid in water and 0.1% formic acid in acetonitrile) were chosen for the optimized method. The final modifications to the method involved optimization of the gradient to improve the resolution between the observed peaks. A sample chromatogram, obtained using the optimized methodology, for the extracted monkey plasma sample is illustrated in FIG. 28.

The optimized HPLC-MS/MS bioanalytical method for the quantitation of the parent in human plasma, summarized below, was qualified.

### Summary of HPLC-MS/MS Conditions Qualified

Analytical Conditions

| Column | ACE 5 C18, 3 pm, 2.1 × 50 mm | | |
|---|---|---|---|
| Mobile Phase A | 0.1% formic acid in water | | |
| Mobile Phase B | 0.1% formic acid in acetonitrile | | |
| | Time (min) | % A | % B |
| Pump Gradient Program | 0.5 | 80 | 20 |
| | 1.0 | 65 | 35 |
| | 3.4 | 40 | 60 |
| | 3.5 | 2 | 98 |
| | 4.5 | 2 | 98 |
| | 4.6 | 80 | 20 |

| | |
|---|---|
| Diluent | 0.1% formic acid, |
| | 30:70 water:acetonitrile |
| Flow (ml/min) | 1.0 |
| Column Temperature (° C.) | Ambient |
| Injection Volume (pl) | 30 |
| Sample Temperature (° C.) | 10 |
| Parent MRM | 389.3 → 195.2 |
| IP 109 (internal std) MRM | 431.2 → 195.2 |
| Retention Time | Parent-2.1, |
| (min) | IP 109-3.7 |
| Run Time (min) | 5.0 |

Calibration standards over a range of 0.5-1,000 ng/mL were prepared and extracted in duplicate as described below.

### Calibration Standard Preparation

| | | | | | | After 100X |
|---|---|---|---|---|---|---|
| | | Spiking | Spiking | Diluent | Final | dilution in |
| | Spiking | Sol Conc. | Sol | (MeOH) | Conc. | plasma |
| Sample ID | Solution Name | (μg/mL) | Vol (μL) | Vol (μL) | (ng/mL) | (ng/mL) |
| Calibration Std 1 | Spiking Std 3 | 1 | 50 | 950 | 50 | 0.5 |
| Calibration Std 2 | Spiking Std 3 | 1 | 100 | 900 | 100 | 1 |
| Calibration Std 3 | Spiking Std 2 | 100 | 5 | 995 | 500 | 5 |
| Calibration Std 4 | Spiking Std 2 | 100 | 10 | 990 | 1,000 | 10 |
| Calibration Std 5 | Spiking Std 2 | 100 | 25 | 975 | 2,500 | 25 |
| Calibration Std 6 | Spiking Std 2 | 100 | 75 | 925 | 7,500 | 75 |
| Calibration Std 7 | Spiking Std 1 | 1,000 | 18 | 982 | 18,000 | 180 |
| Calibration Std 8 | Spiking Std 1 | 1,000 | 20 | 980 | 20,000 | 200 |
| Calibration Std 9 | Spiking Std 1 | 1,000 | 50 | 950 | 50,000 | 500 |
| Calibration Std 10 | Spiking Std 1 | 1,000 | 100 | 900 | 100,000 | 1,000 |

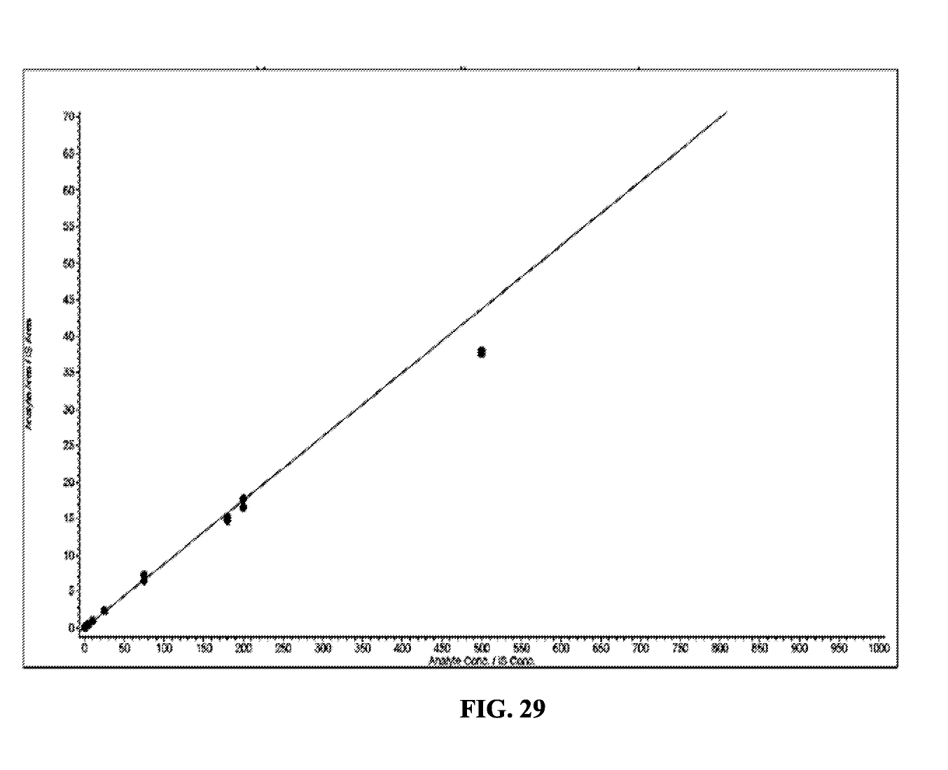

The results of the calibration standard extractions are displayed below. The calibration curve is displayed in FIG. 29.

### Calibration Standard Statistics

| Data | Std1 | Std 2 | Std 3 | Std 4 | Std 5 | Std 6 | Std 7 | Std 8 | Std 9 | Std 10 |
|---|---|---|---|---|---|---|---|---|---|---|
| Expected Conc. | 0.5 | 1 | 5 | 10 | 25 | 75 | 180 | 200 | 500 | 1000 |
| # Of Values Used | 2 of 2 | 2 of 2 | 2 of 2 | 2 of 2 | 2 of 2 | 2 of 2 | 2 of 2 | 2 of 2 | 2 of 2 | 0 of 2 |
| Data Point #1 | 0.475 | 1.04 | 5.26 | 10.4 | 26.2 | 82.5 | 168 | 202 | 435 | 792 |
| Data Point #2 | 0.490 | 1.06 | 5.44 | 10.4 | 26.4 | 73.0 | 173 | 189 | 430 | 784 |
| Mean | 0.483 | 1.05 | 5.35 | 10.4 | 26.3 | 77.7 | 170 | 195 | 432 | N/A |
| Standard Dev. | 0.011 | 0.02 | 0.13 | 0.02 | 0.1 | 6.7 | 4 | 9 | 4 | N/A |
| % CV | 2.3 | 1.7 | 2.4 | 0.2 | 0.3 | 8.6 | 2.3 | 4.8 | 0.9 | N/A |
| Accuracy | 96.5 | 104.9 | 107.0 | 103.9 | 105.2 | 103.6 | 94.7 | 97.6 | 86.4 | N/A |

The optimized method was determined to be linear over the range of 0.5-500 ng/mL of the parent. A total of 18 out of 18 standards over the linear range met the acceptance criteria of back-calculating to within ±15% of their nominal concentrations and had CV's under 15% for each duplicate extraction. Both sets of standards prepared at the lower limit of quantitation (LLOQ) and upper limit of quantitation (ULOQ) met the specified acceptance criteria.

The extractions of blank plasma exhibited a consistent parent signal. The average signal of approximately 20 injections of extracted blank plasma corresponded to a calculated concentration of approximately 0.05 ng/mL, which represented 10% of the lower limit of quantitation (0.5 ng/mL). This blank signal did not affect the quantitation of the parent in human plasma.

The optimized HPLC-MS/MS bioanalytical method was modified to collect data in full scan mode and plasma samples from previous studies were extracted and analyzed. The predicted m/z of likely metabolites was extracted from the full scan data with respect to the mass change from the parent. The potential metabolites that were identified, along with their respective retention times, are listed below for dog and monkey plasma samples. The entries marked with an asterisk represent cases wherein the potential metabolite is likely due to an isotopic distribution from a different species with a similar m/z. These cases were identified based on the co-elution of species with m/z values differing by only 2 units and the corresponding peak intensities of each peak.

### Potential Metabolites Observed in Dog Plasma Samples

| Mass | 1 hr | | 4 hr | | 12 hr | |
|---|---|---|---|---|---|---|
| Change | yes/no | RT (min) | yes/no | RT (min) | yes/no | RT (min) |
| −2 | no | — | yes | 2.1 | yes | 2.1 |
| 2 | no | — | yes | 2.2, 2.4 | yes | 2.2, 2.4 |
| 14 | no | — | yes | 1.4 | yes | 1.4 |
| 16 | yes | 2.0 | yes | 1.5 | yes | 1.5 |
| 18 | yes | 1.1 | yes* | 1.5 | yes* | 1.5 |
| 30 | no | — | no | — | no | — |
| 32 | yes | 1.0, 1.8 | yes | 1.2 | yes | 1.2 |
| 34 | yes | 1.4 | yes | 1.4 | yes | 1.4 |
| 46 | no | — | no | — | no | — |
| 48 | no | — | no | — | no | — |
| 50 | no | — | no | — | no | — |
| 80 | no | — | no | — | no | — |
| 176 | no | — | no | — | no | — |

### Potential Metabolites Observed in Monkey Plasma Samples

| Mass | 1 hr | | 4 hr | | 12 hr | |
|---|---|---|---|---|---|---|
| Change | yes/no | RT (min) | yes/no | RT (min) | yes/no | RT (min) |
| −2 | no | — | yes | 2.1 | yes | 2.1 |
| 2 | no | — | no | — | yes | 2.3 |
| 14 | yes | 1.0 | yes | 1.4 | no | — |
| 16 | yes | 1.3 | yes | 1.3 | yes | 1.3 |
| 18 | yes* | 1.3 | yes* | 1.3 | yes* | 1.3 |
| 30 | no | — | no | — | no | — |
| 32 | no | — | yes | 1.0, 1.2 | no | — |
| 34 | yes | 2.2 | no | — | no | — |
| 46 | no | — | no | — | no | — |
| 48 | no | — | no | — | no | — |
| 50 | no | — | no | — | no | — |
| 80 | yes | 1.5 | yes | 1.5 | yes | 1.5 |
| 176 | no | — | no | — | no | — |

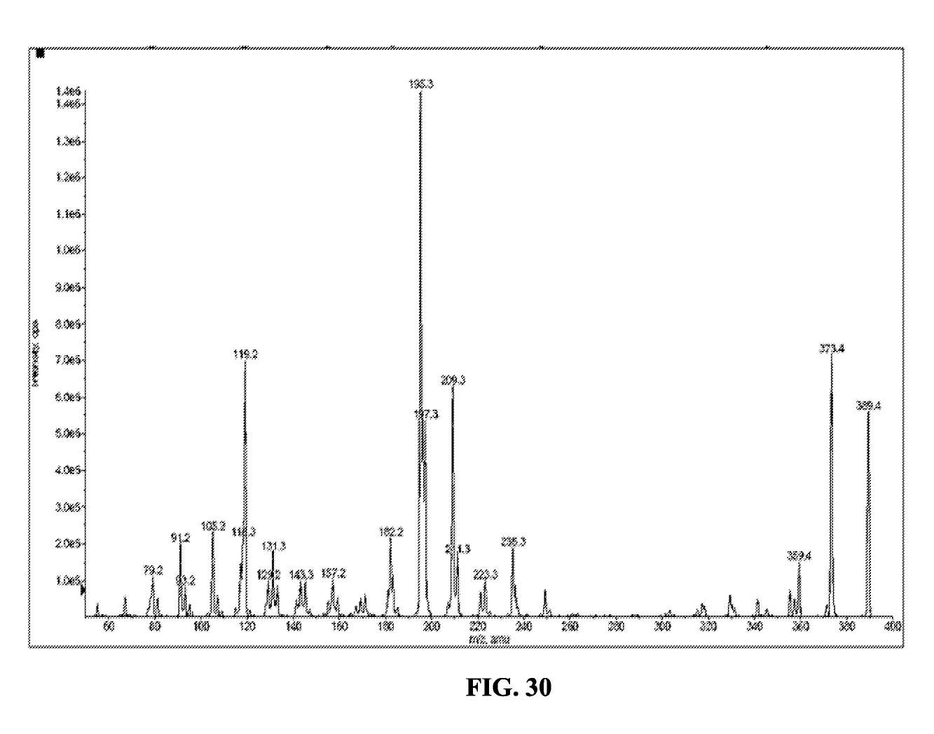

A well-defined fragmentation pattern of the parent drug was obtained by infusing a solution of the parent, prepared in analytical method diluent, directly into the mass spectrometer, and is illustrated in FIG. 30.

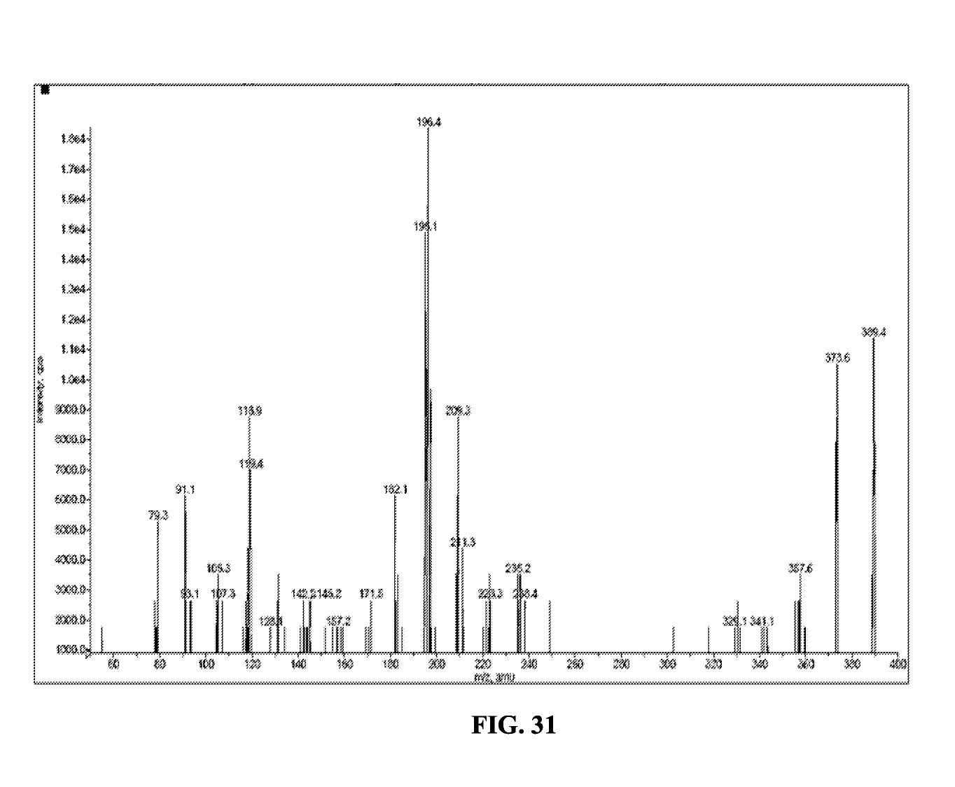

A series of parent standards were prepared in analytical method diluent and analyzed in product ion scan mode to obtain fragmentation patterns. The lowest concentration at which the pattern could be distinguished from background noise was determined to be approximately 0.42 ng/mL of parent prepared in analytical method diluent. The fragmentation pattern is illustrated in FIG. 31.

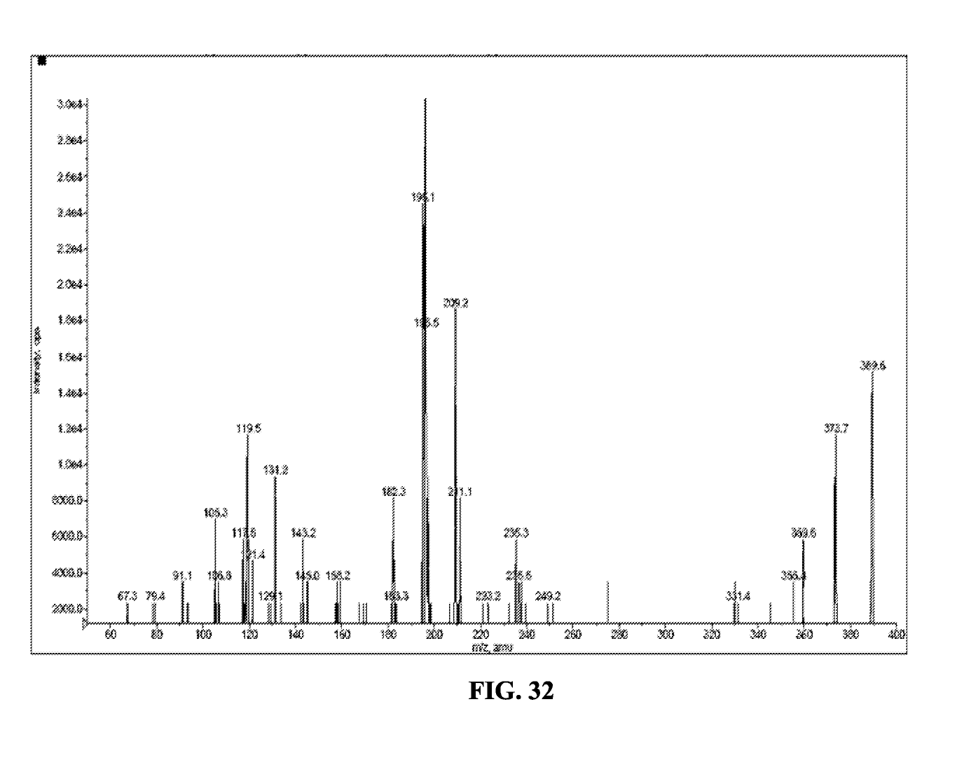

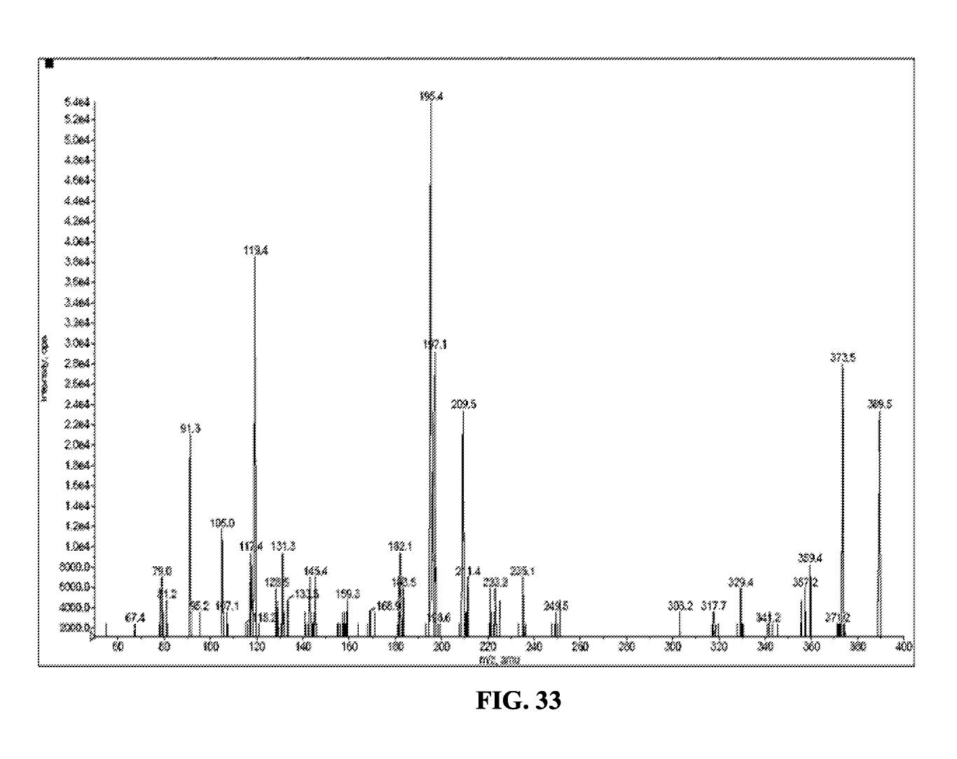

Subsequently, the parent was spiked into plasma and the extraction was performed according to the validated method. The resultant samples of progressively lower concentration were analyzed to determine if it would be feasible to analyze retained in vivo study samples for the presence of defined fragmentation patterns. The lowest concentration plasma sample at which the fragmentation pattern could be distinguished from background noise was determined to range from 5-10 ng/mL of the parent in plasma. The fragmentation patterns for the two concentrations are illustrated in FIGS. 32 and 33. A defined pattern was observed for the 5 ng/mL sample (FIG. 32); however, the low signal strength can be a limiting factor when applied to the in vivo samples.

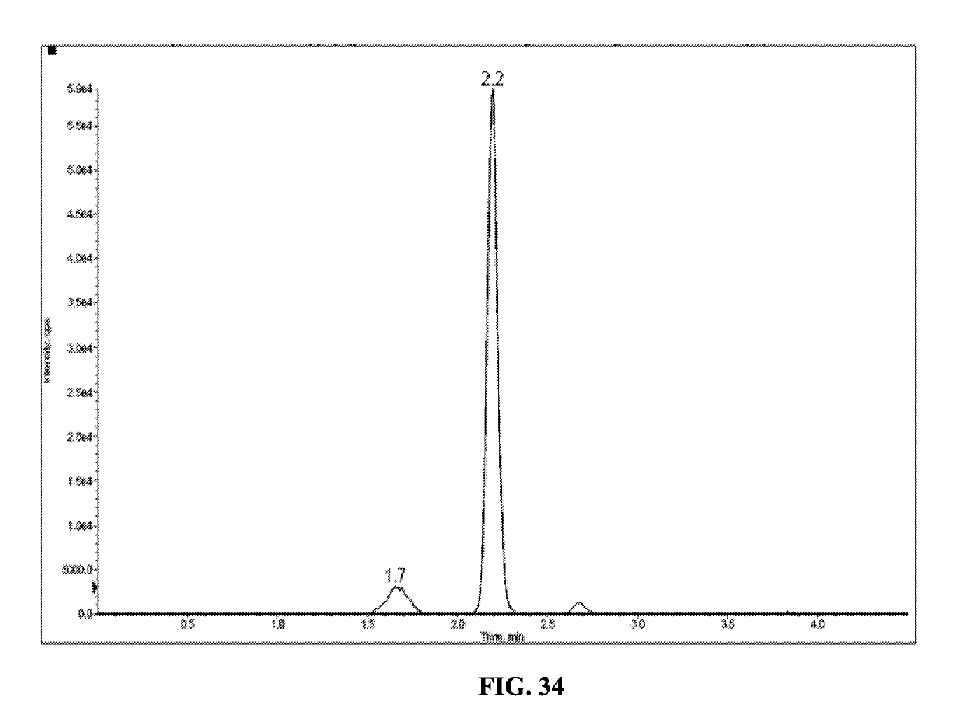

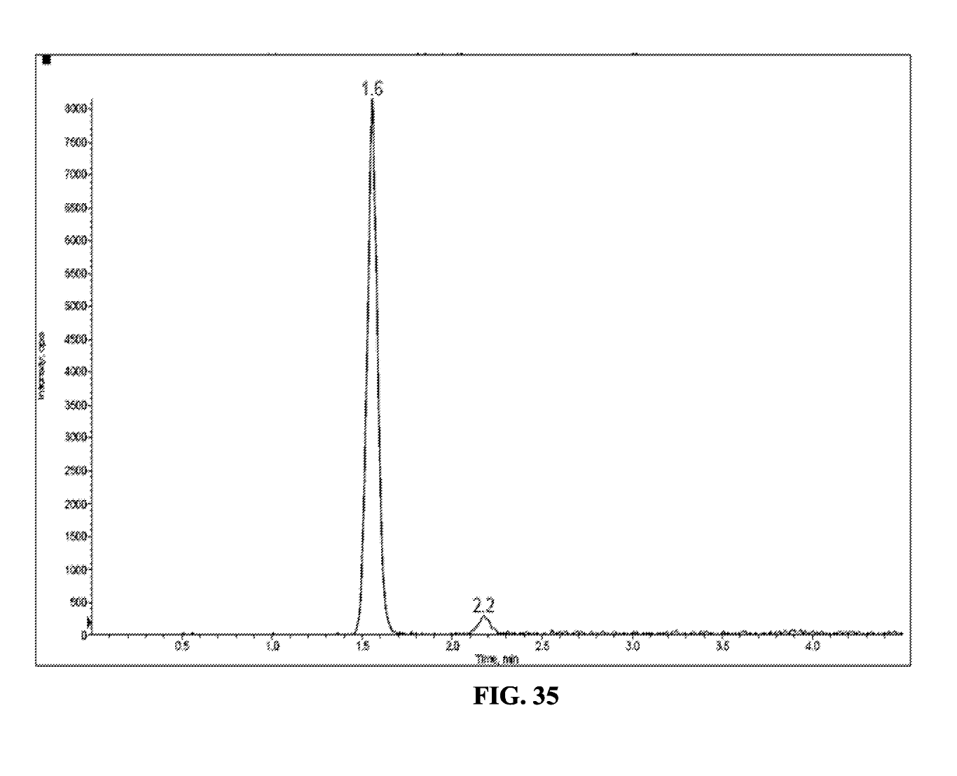

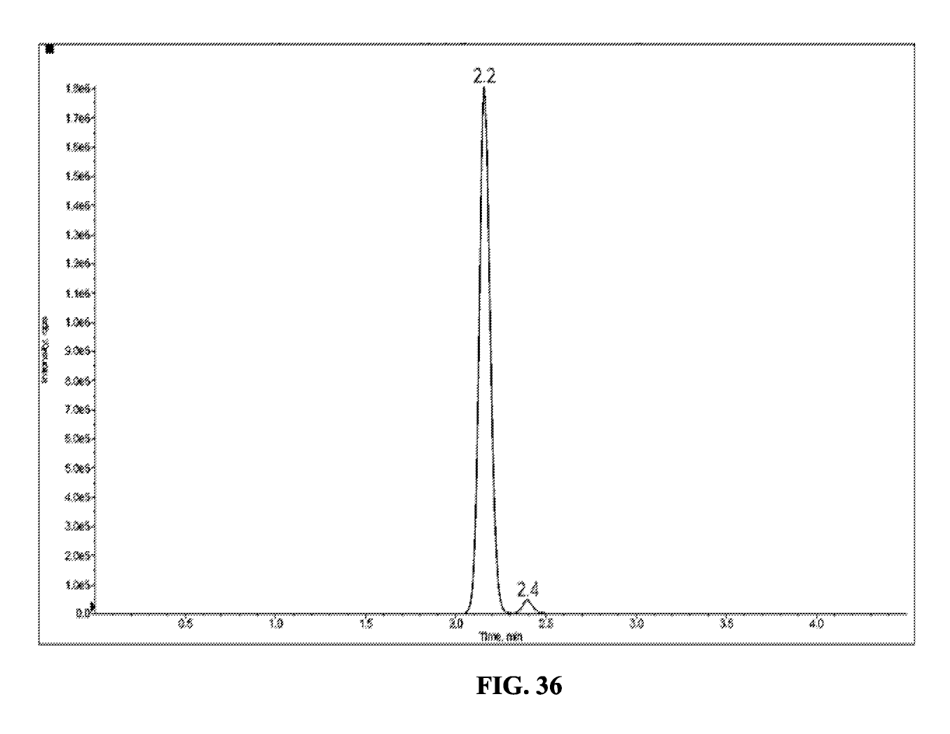

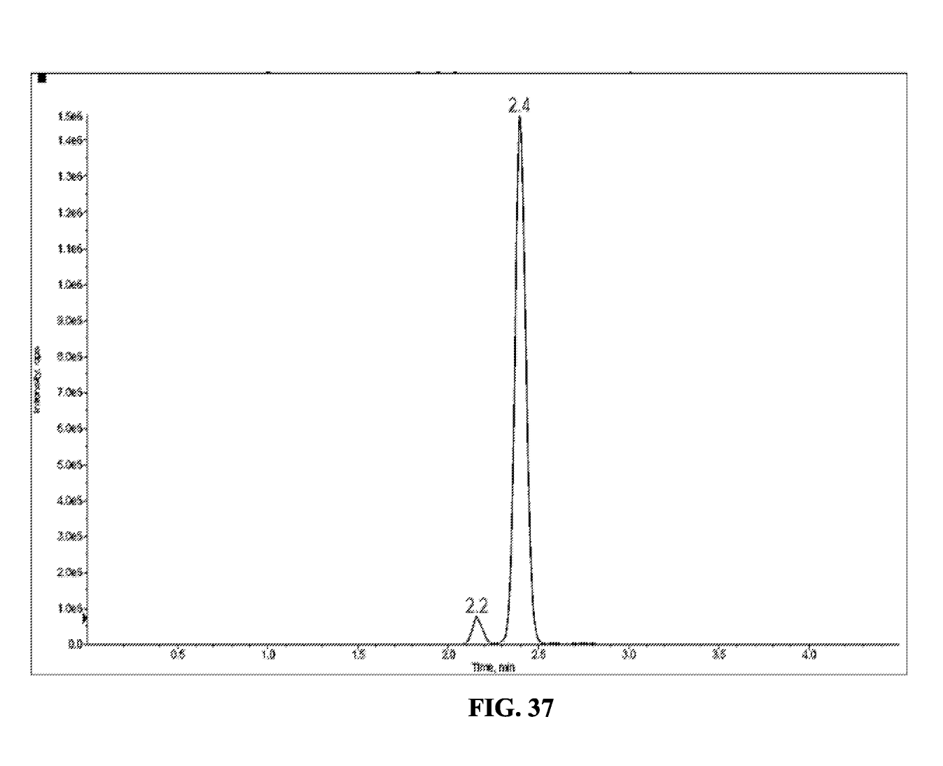

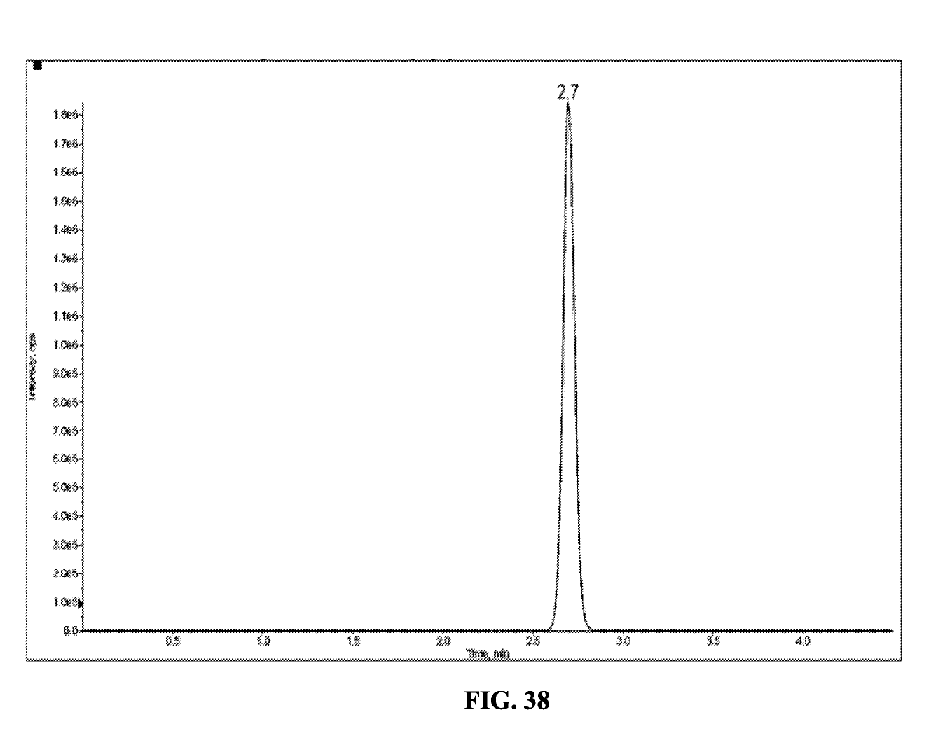

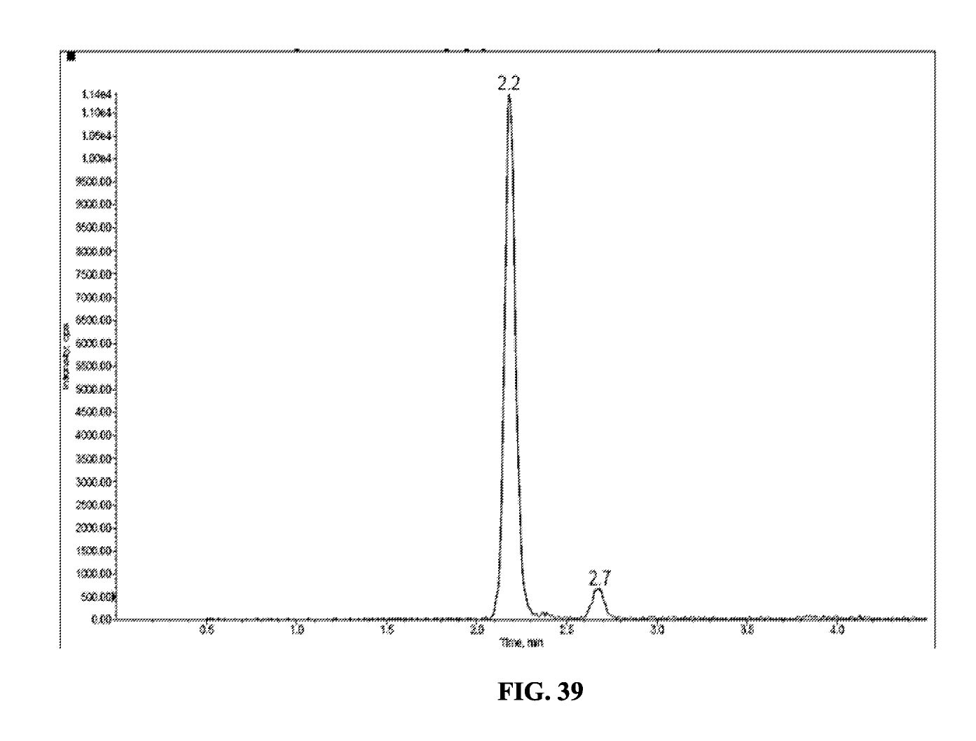

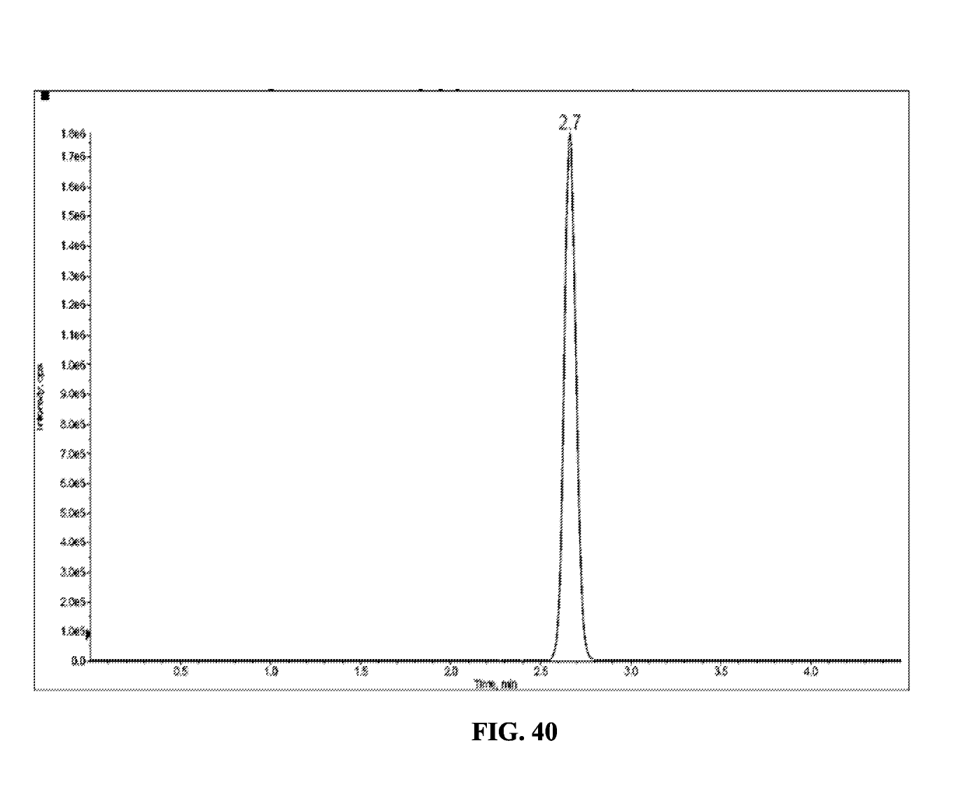

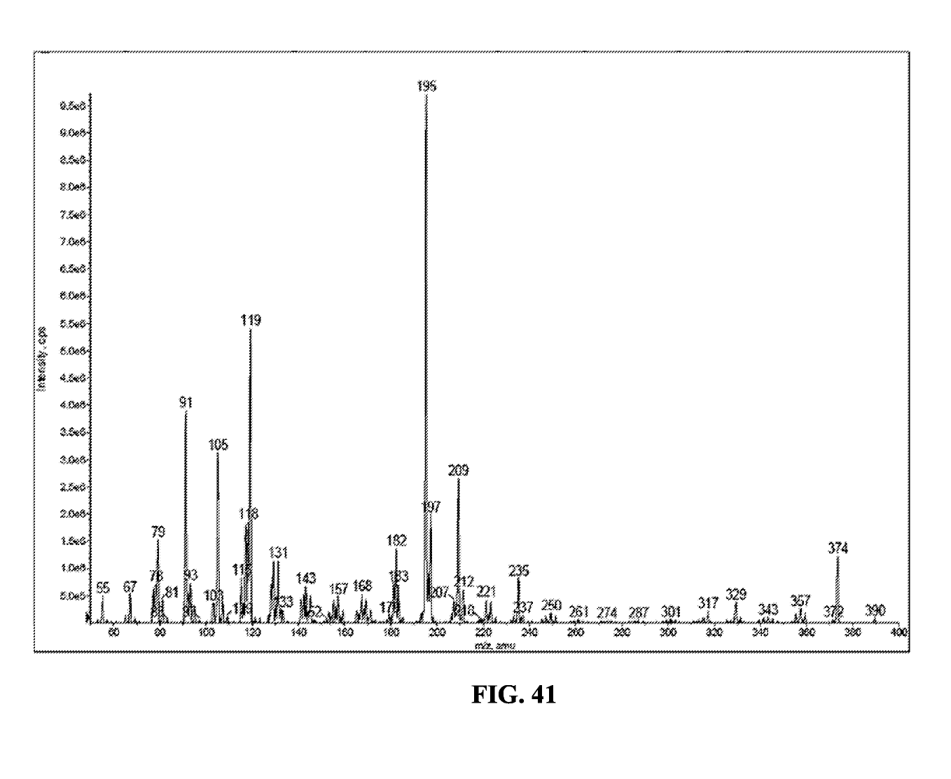

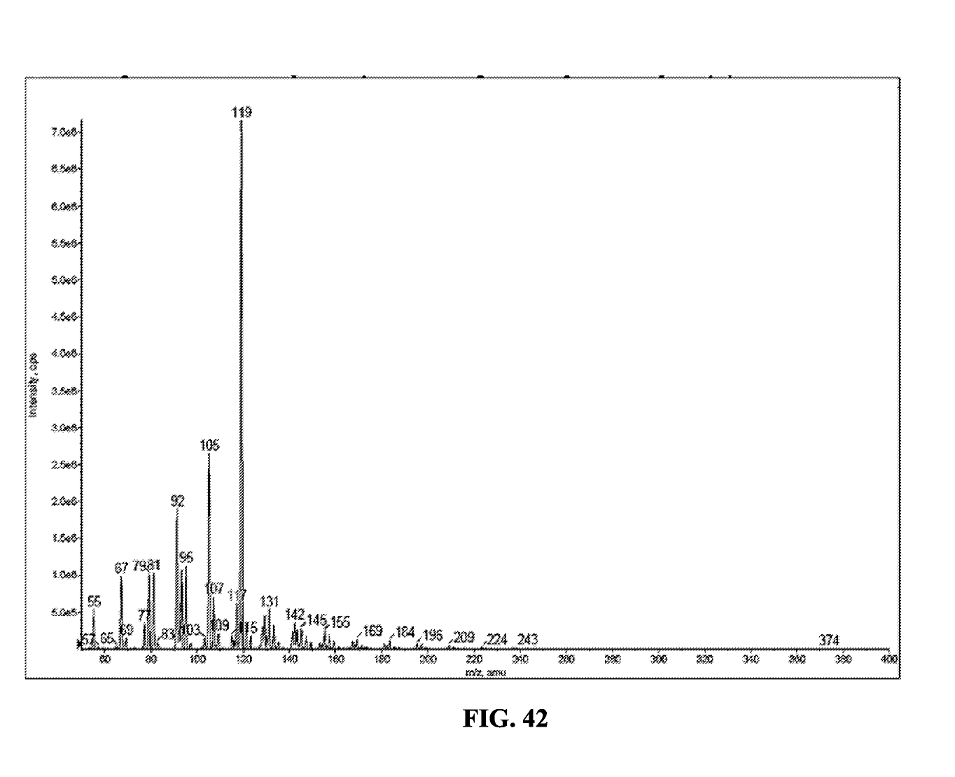

Nine authentic standards of possible metabolites were prepared in method diluent and analyzed by the optimized HPLC-MS/MS method. A summary of the possible metabolites and comparison to extracted cynomolgus monkey plasma is provided below. Chromatograms of the extracted plasma sample and the six of the nine authentic standards with observed peaks are presented in FIGS. 34-40. Three of the nine authentic standards, Std. 4, Std. 8, and Std. 9 were not observed in the method chromatography at the MRM of 391→195, while two other standards, Std. 1 and Std. 6 had only minor peaks at the MRM of 391→195. These five standards were infused and four, Std. 4, Std. 6, Std. 8, and Std. 9, were determined to have molecular ion masses of 2 amu greater than that of the parent compound. Compound Std. 1 was determined to have a molecular mass of 388 amu, the same as that of the parent compound, although under the optimized MS/MS parameters the m/z 195 fragment was not produced in significant quantity. A comparison of the product ion spectra of the parent compound and Std. 1, obtained using the MS/MS parameters optimized for the parent, is provided in FIGS. 41 and 42.

### Summary of Possible Metabolites

| | Observed Peaks, RT (min) | | | | | |
|---|---|---|---|---|---|---|
| Compound | 1.6 | 1.7 | 2.2 | 2.4 | 2.7 | Notes |
| Cyno Plasma | | Minor | Major | | Minor | Parent compound peak at 2.2 min |
| Std. 1 | Minor | | Minor | | | Minimal response with MRM for Parent compound |
| 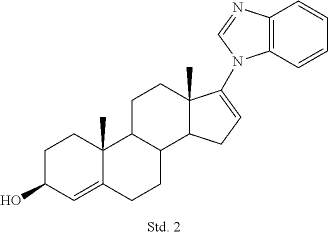 | | | Major | Minor | | |
| Std. 3 | | | Minor | Major | | |
| Std. 4 | | | | | | Molecular ion of +2 amu to Parent compound |
| 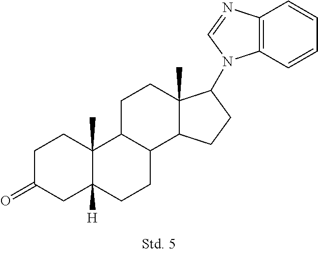 | | | | | Major | |
| Std. 6 | | | Minor | | Minor | Molecular ion of +2 amu to Parent compound |
| 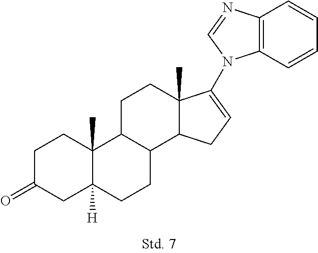 | | | | | Major | |
| Std. 8 | | | | | | Molecular ion of +2 amu to Parent compound |
| Std. 9 | | | | | | Molecular ion of +2 amu to Parent compound |

None of the authentic standards matched the chromatography of the early eluting metabolite observed in the monkey samples at 1.7 minutes.

Two of the authentic metabolites, Std. 5 and Std. 7, did match the chromatography of the later eluting metabolite at 2.7 minutes.

The authentic standard of Std. 2 was observed to co-elute (most prominent peak) with the parent compound. If this potential metabolite was present in the plasma samples it would not be distinguishable from the parent in the current method.

The authentic standards Std. 1 and Std. 3 had unique peaks not observed in the chromatography of plasma samples. The authentic standard of Std. 1 was observed to have a parent mass matching that of the parent of 388 amu, although with the MS/MS parameters optimized for the parent the m/z 195 fragment was not produced in significant quantity (FIGS. 41 and 42), rather the most prominent fragment was m/z 119.

The authentic standards Std. 4, Std. 6, Std. 8 and Std. 9 were observed to have parent masses of 390 amu, 2 amu greater than that of the parent. The authentic standard of Std. 6 was observed to have minor peaks that chromatographically co-elute with the parent and with the later eluting metabolite at 2.7 minutes although with a significantly lower response, which combined with an observed parent mass 2 amu greater than the parent suggests that these two peaks are from impurities in the Std. 6 sample.

What is claimed is:

1. A compound or a pharmaceutically acceptable salt or N-oxide of a compound having the structure of Formula (1) 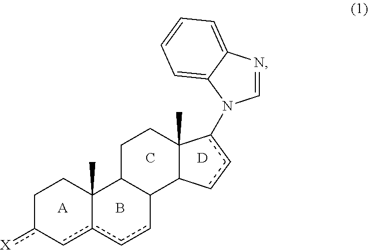
wherein,
(a) the ABCD ring structure and/or one or both methyl groups are independently optionally substituted with one or more substituents selected from C₁-C₆-alkyl, halogenated C₁-C₆-alkyl, C₁-C₆-alkenyl, halogenated C₁-C₆-alkenyl, halogen, amino, aminoalkylene, hydroxyimino, n,n+1-epoxy, carbonyl (oxo), glucuronido, glucuronato, O-linked sulfate, and hydroxy;
(b) X is glucuronido, glucuronato, O-linked sulfate, OH or O; and
(c) dashed lines are taken at each occurrence independently to be double or single bonds, wherein the compound is not: 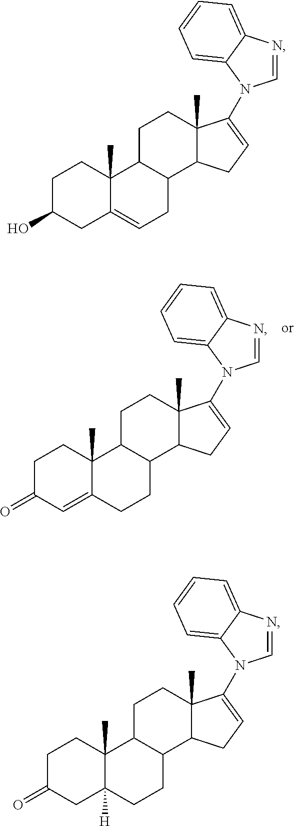
and wherein the compound is formed in vivo after administration of a drug to a subject.

2. The compound of claim 1, wherein X is OH.

3. The compound of claim 1, wherein the compound having the structure of Formula (1) is selected from 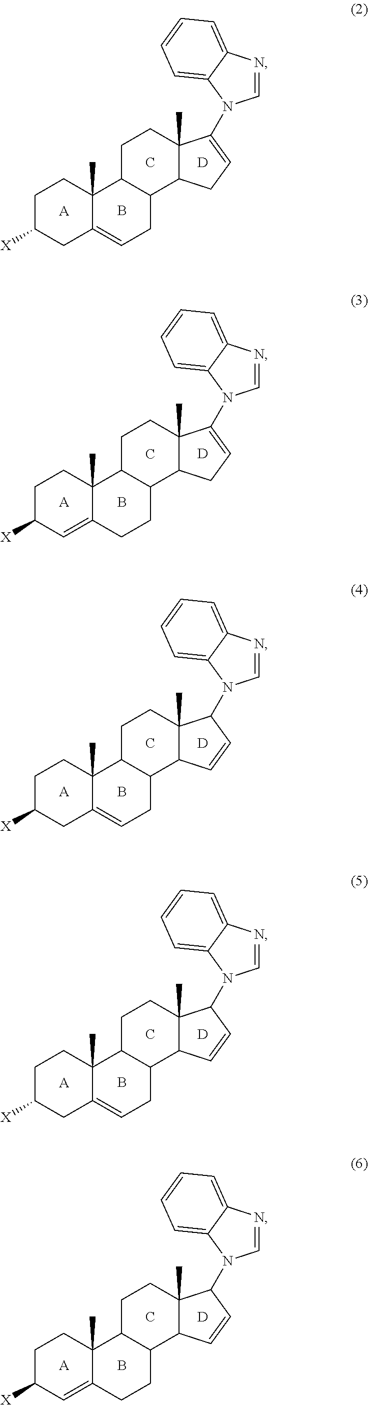 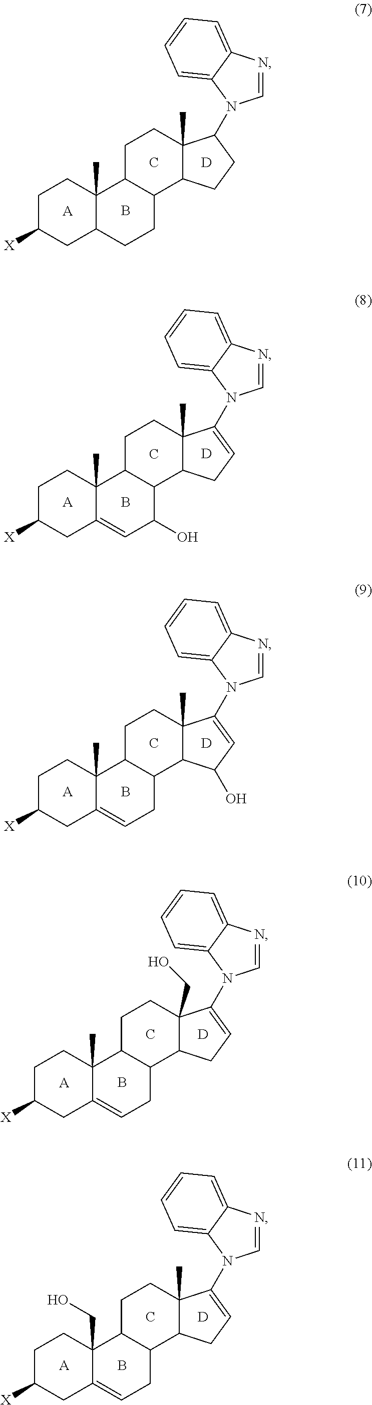 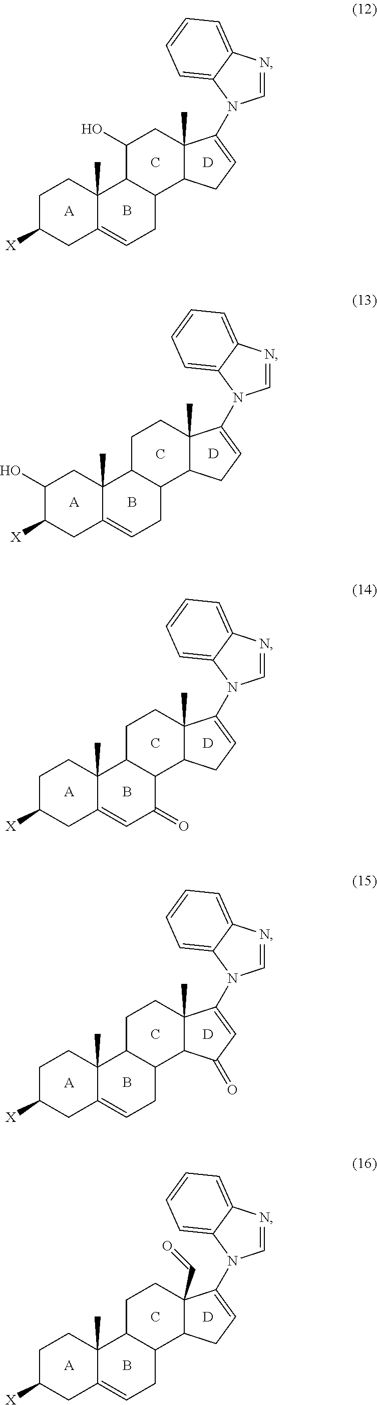 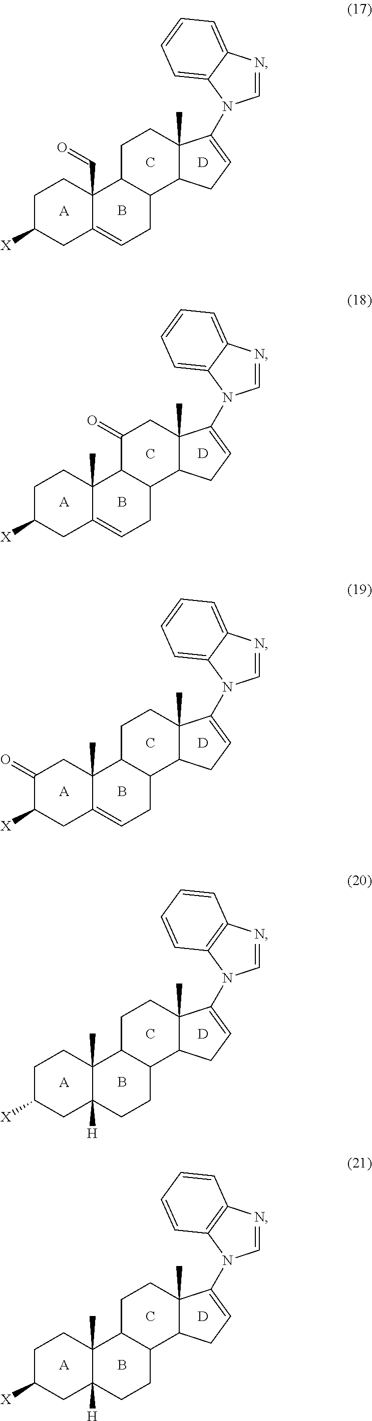 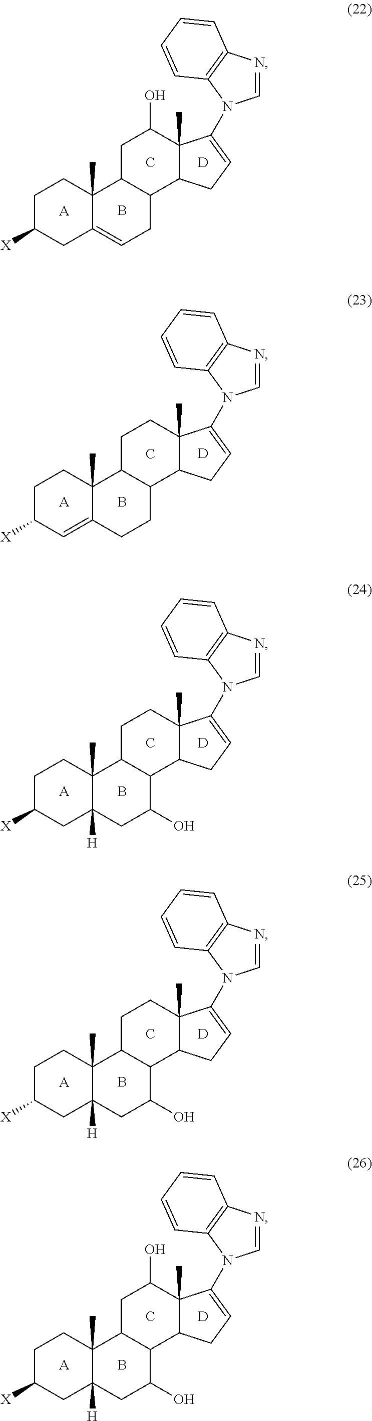 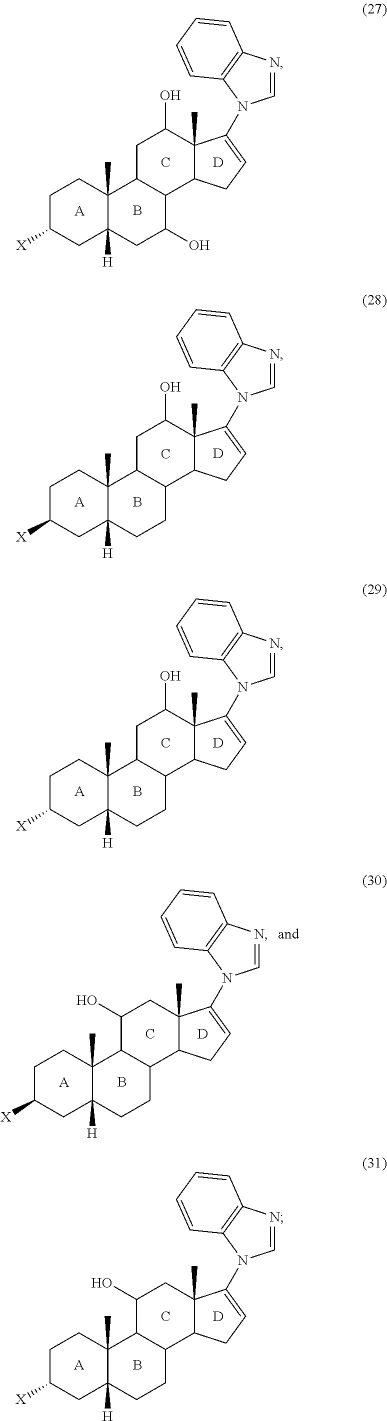
wherein X is glucuronido, glucuronato, or O-linked sulfate.

4. The compound of claim 1, wherein the compound having the structure of Formula (1) is selected from

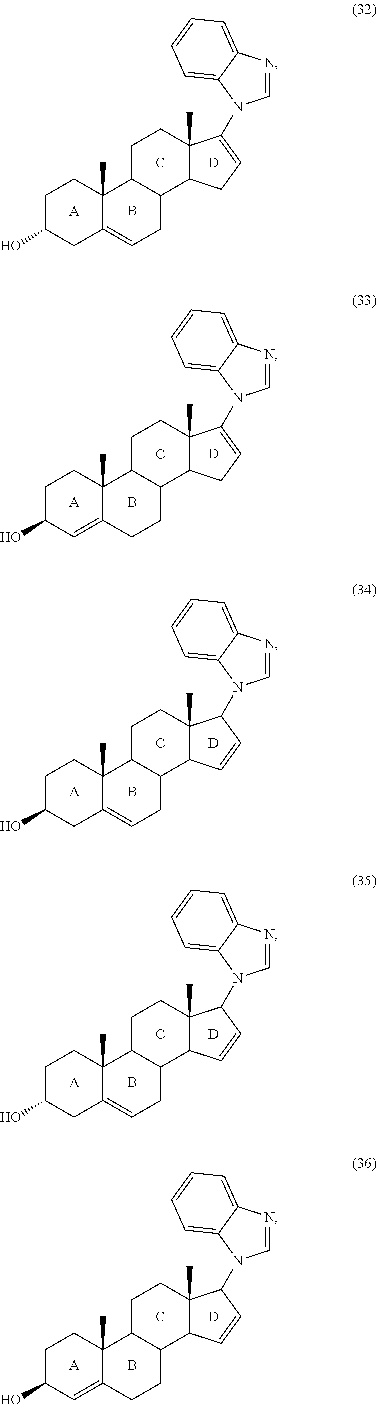

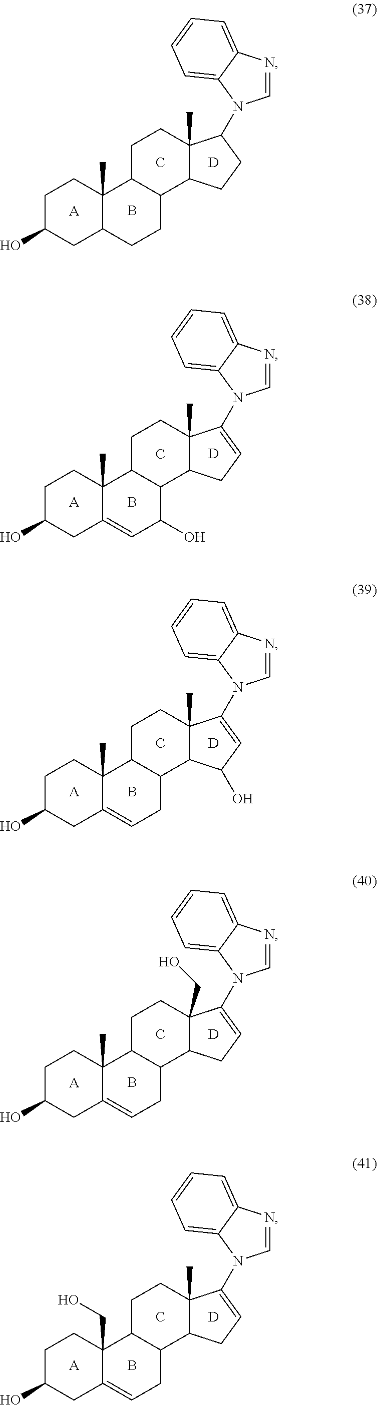

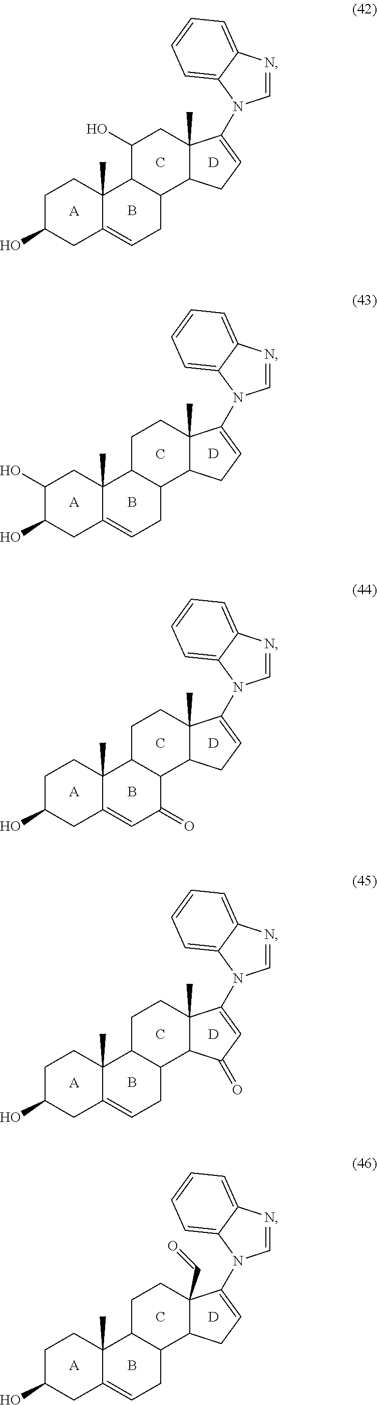

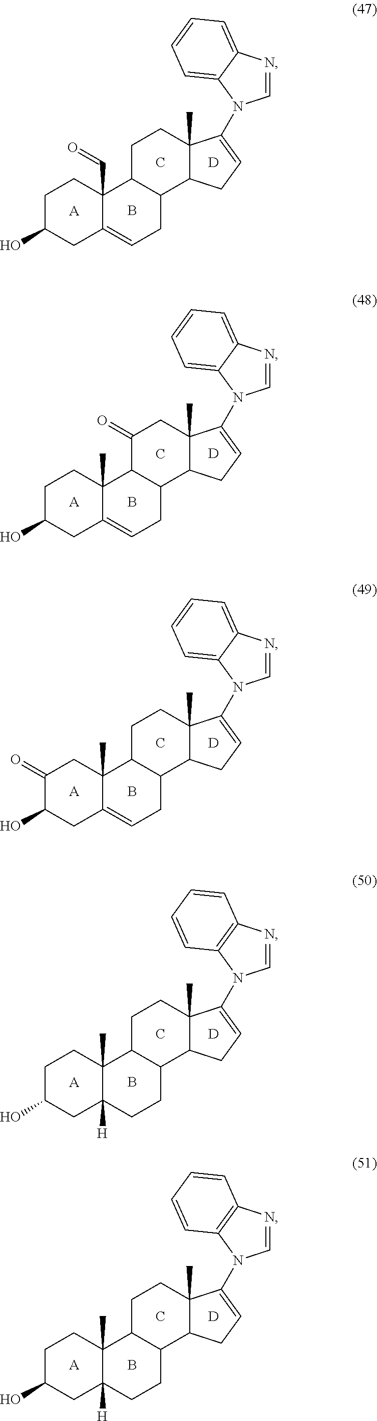

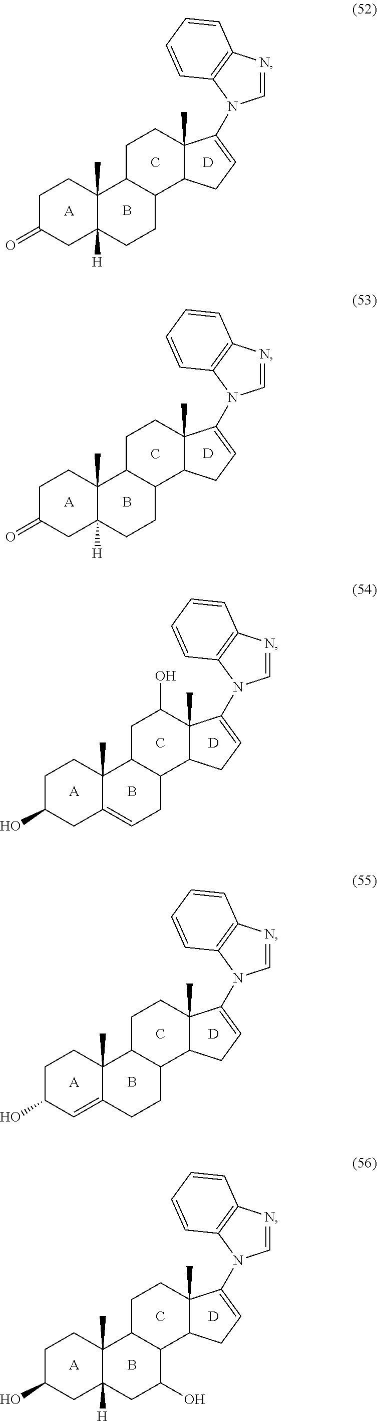

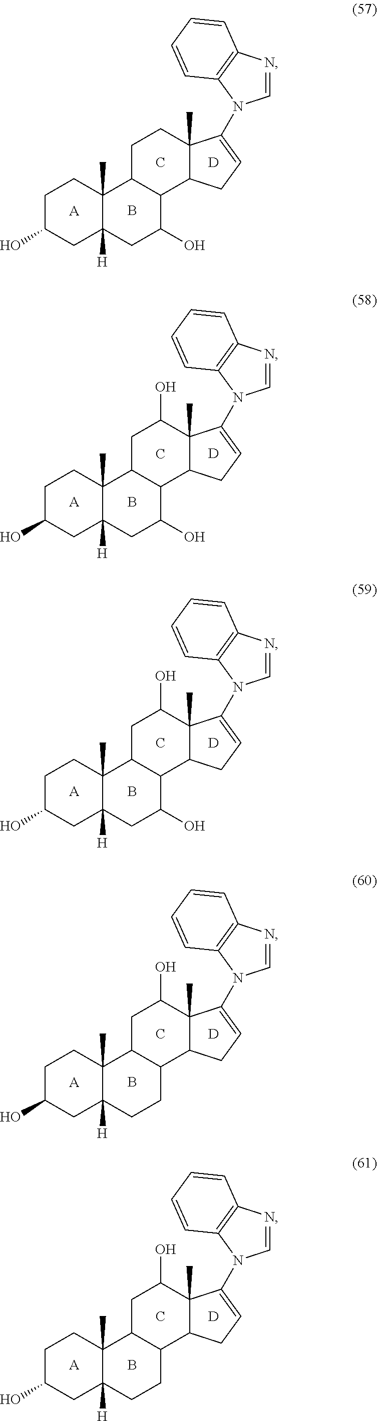

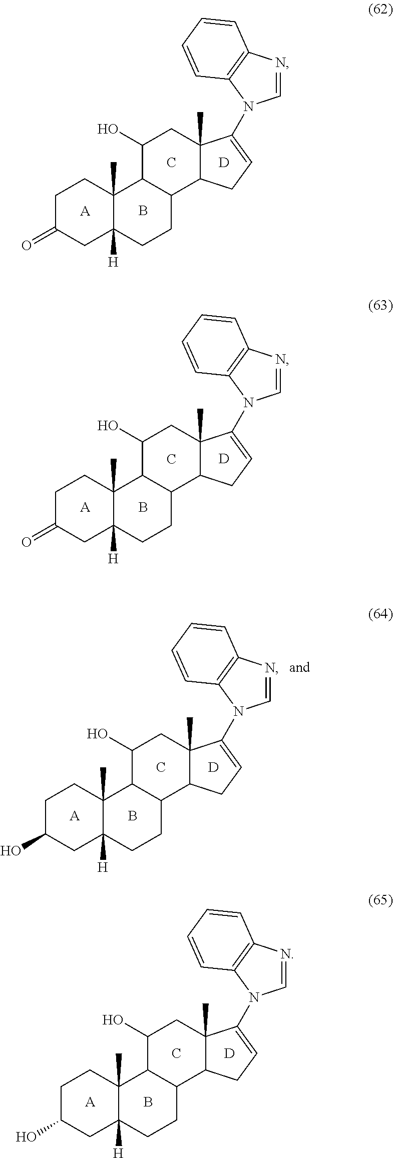

5. A pharmaceutical composition comprising an effective amount of a compound, wherein after administration of the composition to a subject, the compound produces a metabolite or a pharmaceutically acceptable salt or N-oxide thereof of Formula (1): 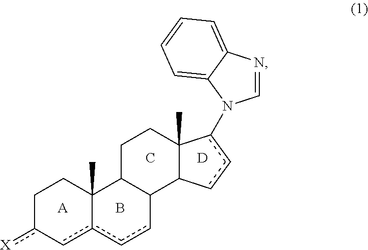
wherein,
(a) the ABCD ring structure and/or one or both methyl groups are independently optionally substituted with one or more substituents selected from C₁-C₆-alkyl, halogenated C₁-C₆-alkyl, C₁-C₆-alkenyl, halogenated C₁-C₆-alkenyl, halogen, amino, aminoalkylene, hydroxyimino, n,n+1-epoxy, carbonyl (oxo), glucuronido, glucuronato, O-linked sulfate, and hydroxy;
(b) X is glucuronido, glucuronato, O-linked sulfate, OH or O; and
(c) dashed lines are taken at each occurrence independently to be double or single bonds, wherein neither the compound nor the metabolite are: 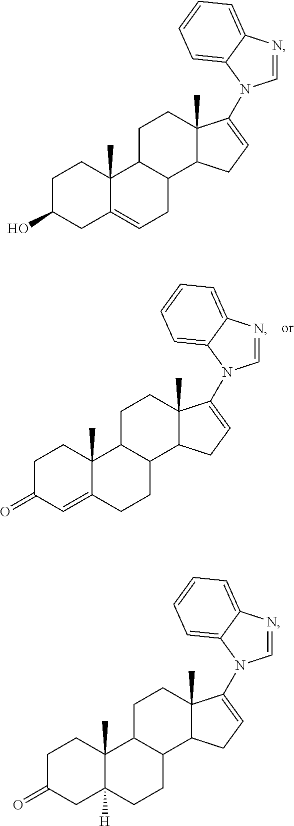
wherein the metabolite is effective for treating an androgen receptor mediated disease or condition.

6. The pharmaceutical composition of claim 5, wherein the compound having the structure of Formula (1) is selected from 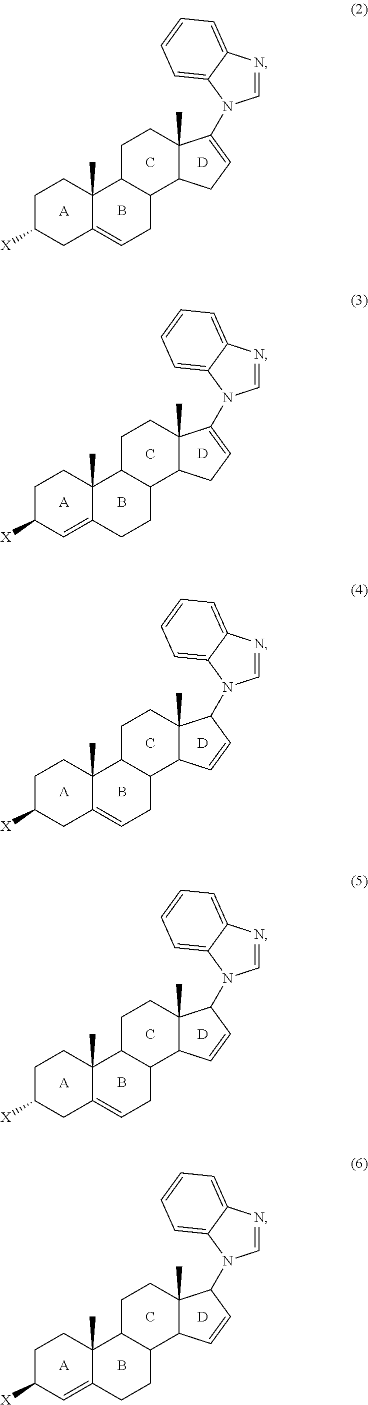 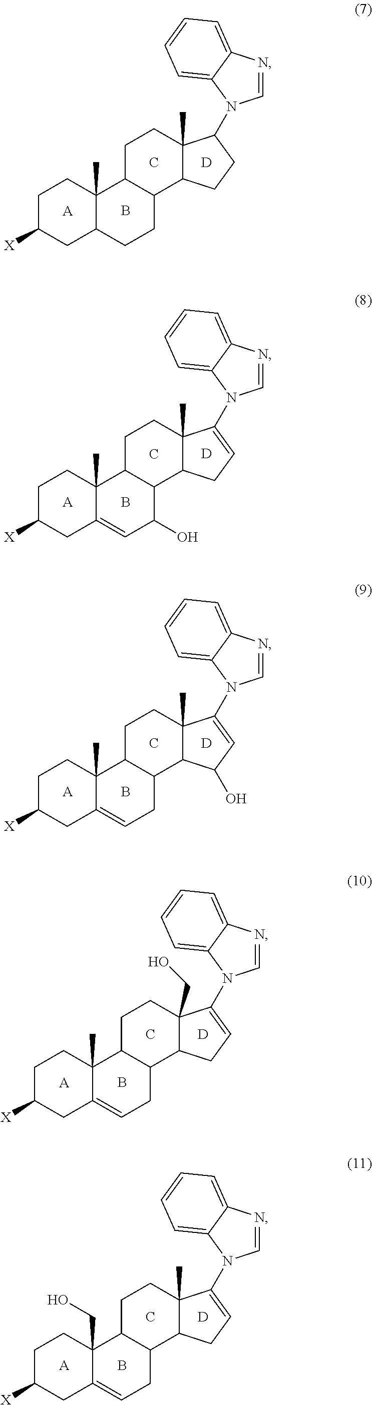 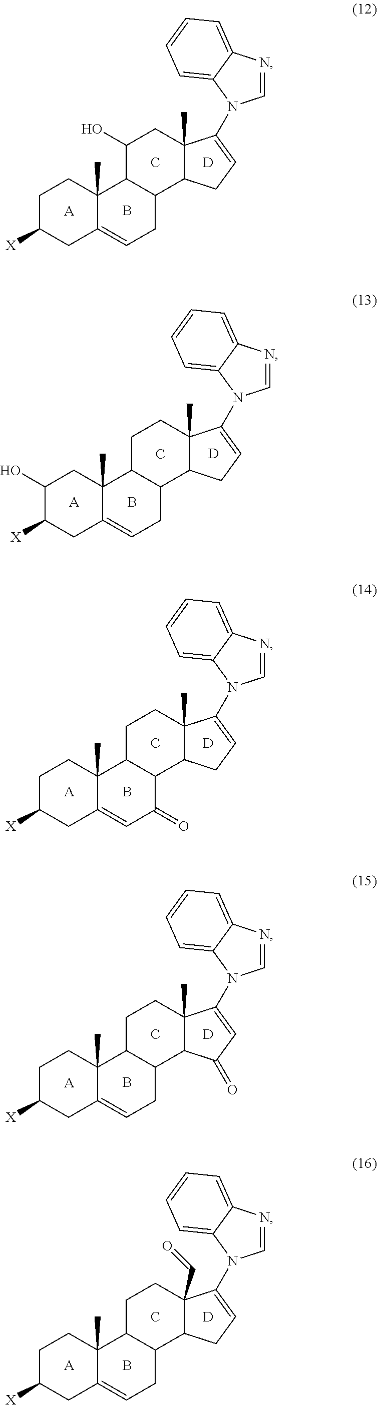 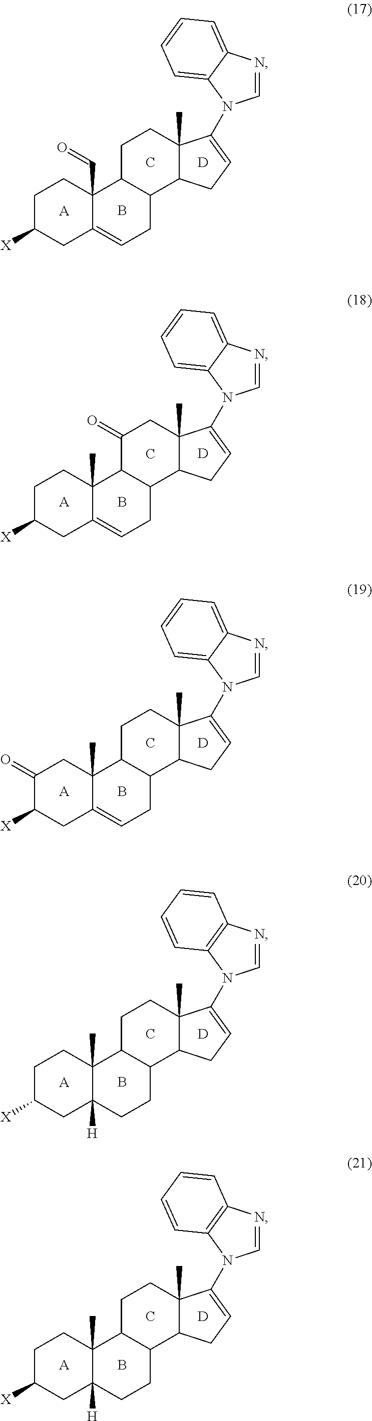 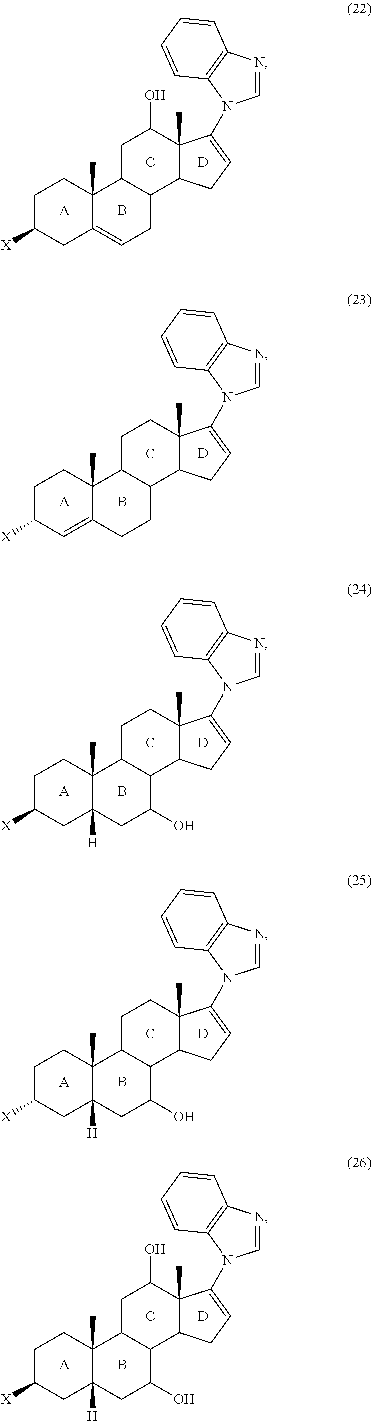 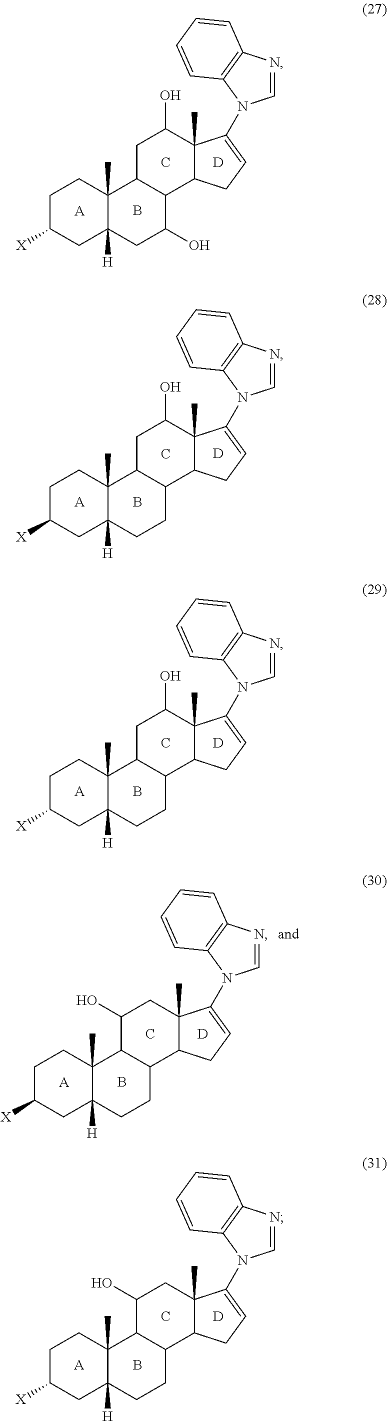
wherein, X is glucuronido, glucuronato, or O-linked sulfate.

7. The pharmaceutical composition of claim 5, wherein the compound having the structure of Formula (1) is selected from

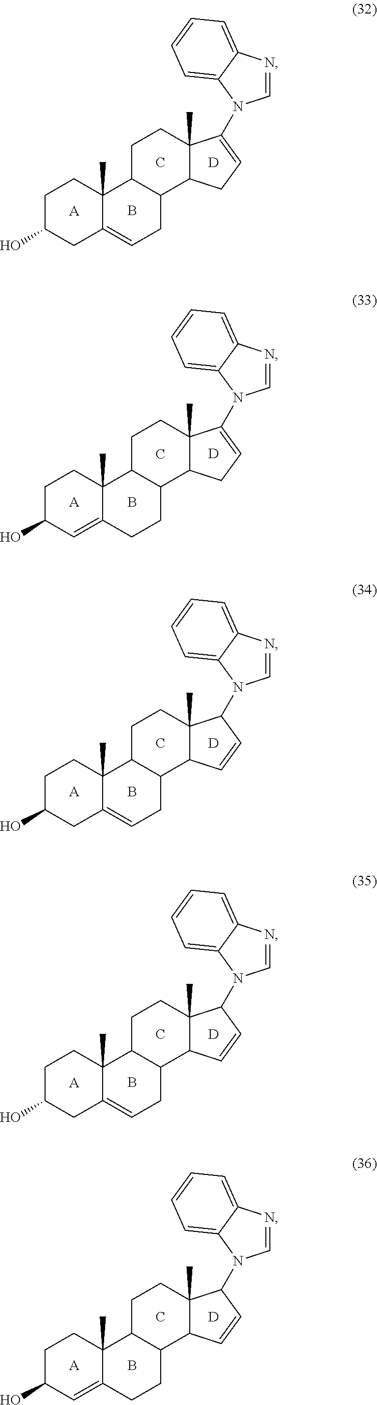

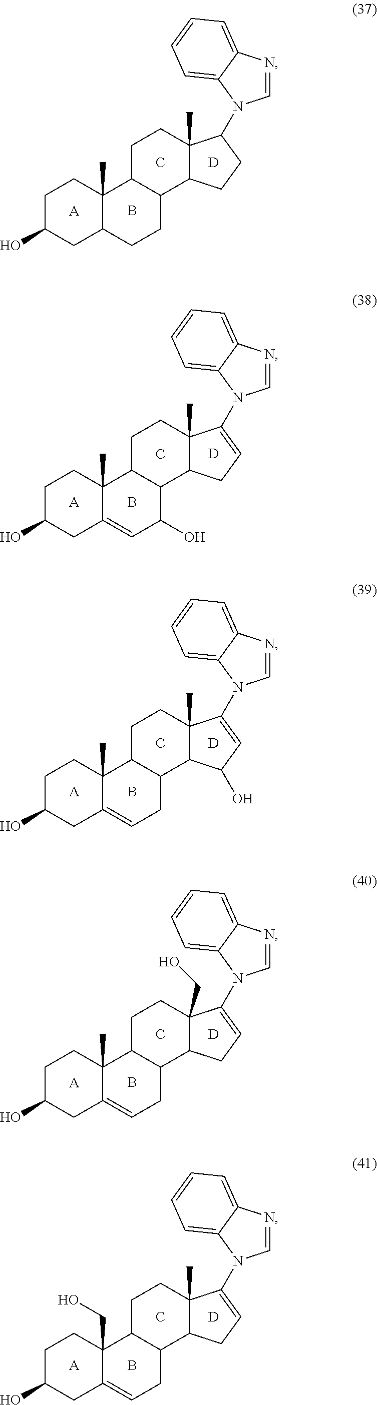

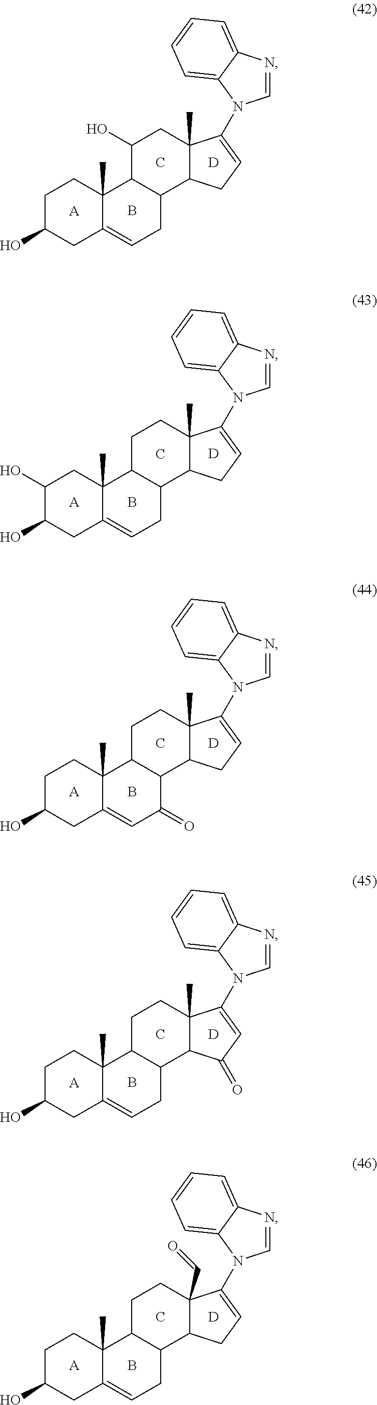

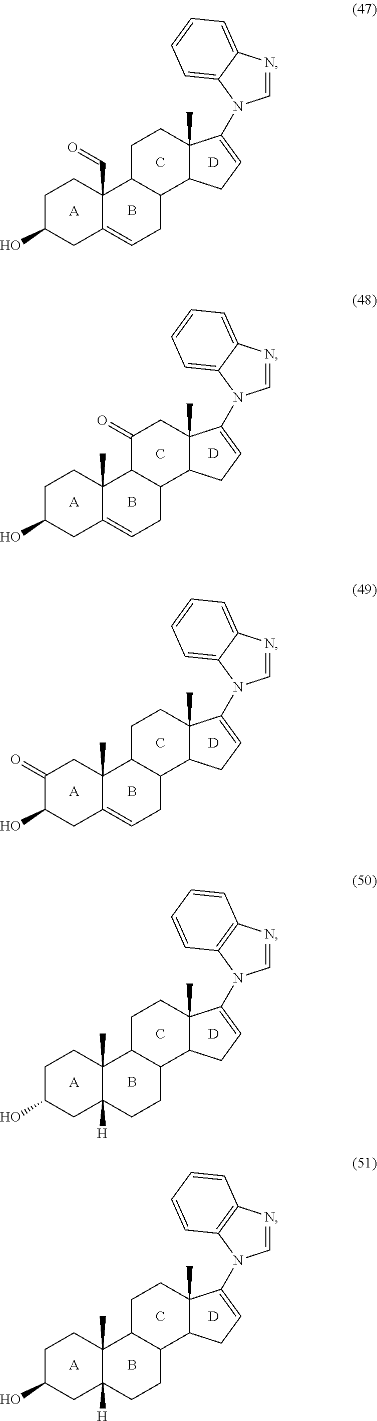

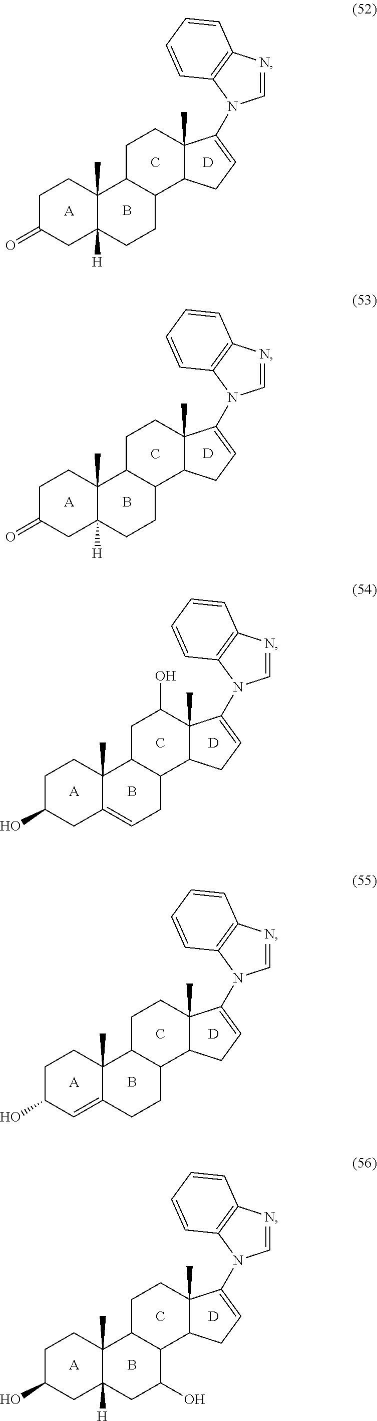

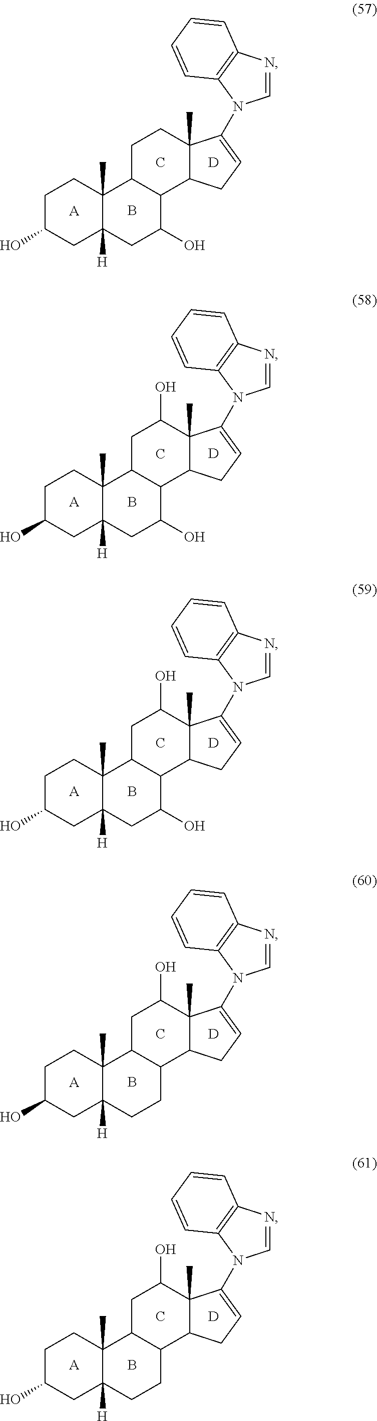

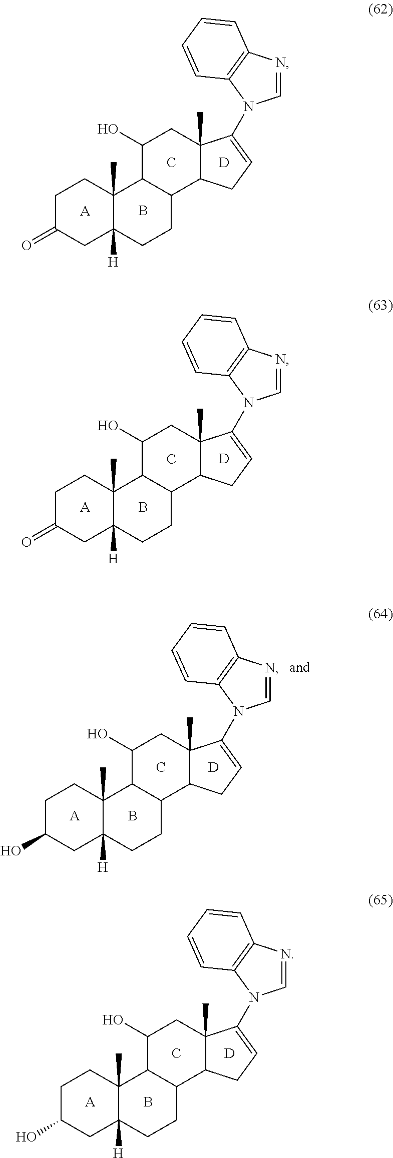

8. The pharmaceutical composition of claim 5 wherein said treating comprises inhibiting androgen biosynthesis, inhibiting androgen receptor signaling or decreasing androgen receptor sensitivity.

9. The pharmaceutical composition of claim 8 wherein said androgen biosynthesis inhibition comprises inhibiting the activity of cytochrome C17α-hydroxylase/C17,20-lyase (CYP17).

10. The pharmaceutical composition of claim 8 wherein said androgen receptor signaling inhibition comprises competitive inhibition of testosterone binding.

11. The pharmaceutical composition of claim 8 wherein said decrease in androgen receptor sensitivity comprises a reduction of the content of androgen receptor protein within the cell, and a diminished ability of the cell to be sustained by low levels of androgenic growth signals.

12. The pharmaceutical composition of claim 5 wherein said treating comprises inhibiting androgen biosynthesis, inhibiting androgen receptor signaling and decreasing androgen receptor sensitivity.

13. The pharmaceutical composition of claim 5 wherein the composition is administered parenterally, intravenously, intramuscularly, intradermally, subcutaneously, intraperitoneally, orally, buccally, sublingually, mucosally, rectally, transcutaneously, transdermally, ocularly, or by inhalation.

14. The pharmaceutical composition of claim 5 wherein the composition is administered as a tablet, a capsule, a cream, a lotion, an oil, an ointment, a gel, a paste, a powder, a suspension, an emulsion, or a solution.

15. The pharmaceutical composition of claim 5 wherein the composition is administered as a capsule.

16. The pharmaceutical composition of claim 15 wherein said capsule comprises the compound as a powder.

17. The pharmaceutical composition of claim 16 wherein said powder is micronized.

18. The pharmaceutical composition of claim 16 wherein said capsule comprises from about 50 mg to about 500 mg of the compound.

19. The pharmaceutical composition of any of claims 15-18 wherein said capsule is administered to a patient, one, two, three, four, five, six, seven, eight, nine, or ten times per day.

20. The pharmaceutical composition of any of claims 15-18 wherein said capsule is administered to a patient for the treatment of prostate cancer.

21. The pharmaceutical composition of any of claims 15-18 wherein said capsule is administered to a patient for the treatment of castration resistant prostate cancer.

22. The pharmaceutical composition of claim 5 wherein the composition further comprises one or more pharmaceutically acceptable excipients.

23. The pharmaceutical composition of claim 22 wherein the excipient comprises a filler, a disintegrant, a lubricant, a surfactant, a glidant, a binder, a sugar, a starch, a varnish, or a wax.

24. The pharmaceutical composition of claim 5 wherein said effective amount of the compound comprises a pharmaceutically acceptable salt, N-oxide, prodrug, crystalline polymorph, or solvate.

25. The pharmaceutical composition of claim 5 wherein said androgen receptor mediated disease or condition is selected from the group consisting of prostate cancer, benign prostatic hyperplasia, hirsutism, alopecia, anorexia nervosa, breast cancer, and male hypergonadism.

26. The pharmaceutical composition of claim 5 wherein said androgen receptor mediated disease or condition is prostate cancer.

27. The pharmaceutical composition of claim 26 wherein said prostate cancer is castration resistant prostate cancer.

28. A method of treating an androgen receptor mediated disease or condition, which method comprises administering to a patient in need thereof a therapeutically effective amount of a compound or a pharmaceutically acceptable salt or N-oxide thereof to inhibit androgen biosynthesis, inhibit androgen receptor signaling and decrease androgen receptor sensitivity, wherein said compound produces a metabolite or a pharmaceutically acceptable salt or N-oxide thereof after administration of the compound to a subject, wherein the metabolite has the structure of Formula (1), 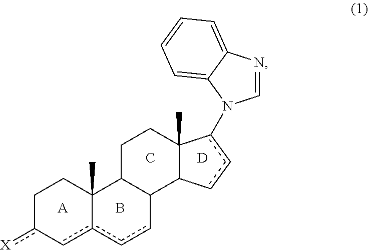
wherein,
(a) the ABCD ring structure and/or one or both methyl groups are independently optionally substituted with one or more substituents selected from C₁-C₆-alkyl, halogenated C₁-C₆-alkyl, C₁-C₆-alkenyl, halogenated C₁-C₆-alkenyl, halogen, amino, aminoalkylene, hydroxyimino, n,n+1-epoxy, carbonyl (oxo), glucuronido, glucuronato, O-linked sulfate, and hydroxy;
(b) X is glucuronido, glucuronato, O-linked sulfate, OH or O; and
(c) dashed lines are taken at each occurrence independently to be double or single bonds, wherein neither the compound nor the metabolite is:

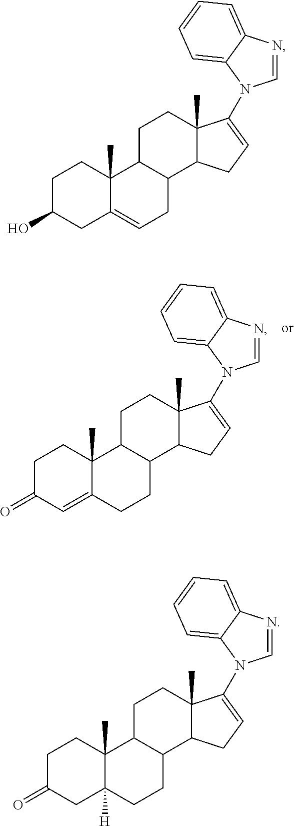

29. The method of claim 28 wherein said androgen receptor mediated disease or condition is selected from the group consisting of prostate cancer, benign prostatic hyperplasia, hirsutism, alopecia, anorexia nervosa, breast cancer, and male hypergonadism.

30. The method of claim 28 wherein said androgen receptor mediated disease or condition is prostate cancer.

31. The method of claim 30 wherein said prostate cancer is castration resistant prostate cancer.

32. The method of claim 28 further comprising administration of a therapeutically effective amount of a second substance.

33. A compound or a pharmaceutically acceptable salt or N-oxide of a compound having the structure of Formula (1) 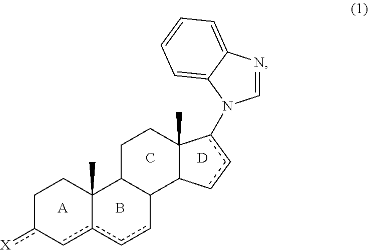
wherein,
(a) the ABCD ring structure and/or one or both methyl groups are independently substituted with two substituents selected from n,n+1 epoxy, oxo, and hydroxy;
(b) X is glucuronido, glucuronato, O-linked sulfate, OH or O; and
(c) dashed lines are taken at each occurrence independently to be double or single bonds, wherein the compound is not: 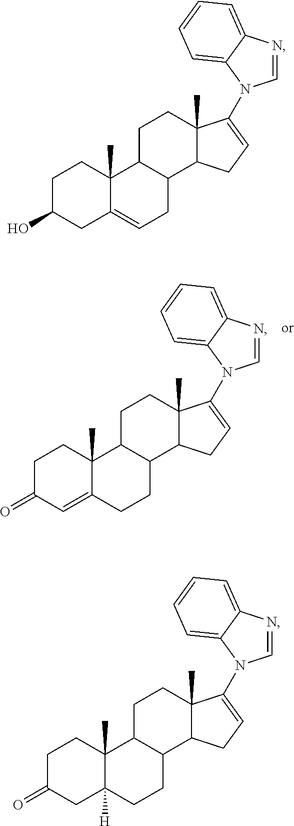
and wherein the compound is formed in vivo after administration of a drug to a subject.

34. A compound or a pharmaceutically acceptable salt or N-oxide of a compound having the structure of Formula (1) 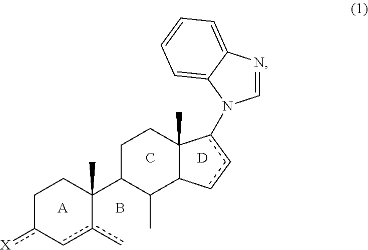
wherein,
(a) the ABCD ring structure and one methyl group is independently substituted with a substituent selected from n,n+1 epoxy, oxo, and hydroxy;
(b) X is glucuronido, glucuronato, O-linked sulfate, OH or O; and
(c) dashed lines are taken at each occurrence independently to be double or single bonds, wherein the compound is not: 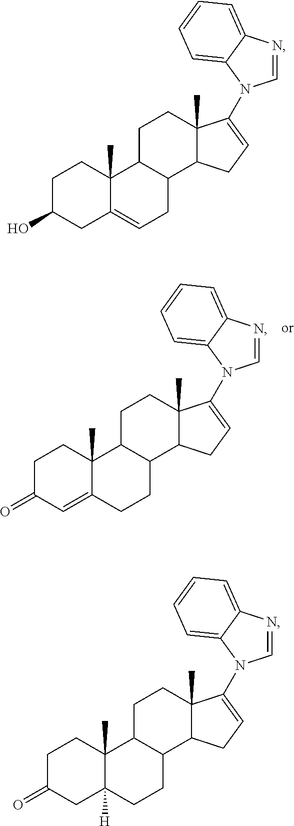
and wherein the compound is formed in vivo after administration of a drug to a subject.
